SEQUENCE LISTING (5 sequences)

SEQ ID NO: 1 - protein, 222 aa, Artificial Sequence
  Description of Artificial Sequence Synthetic polypeptide
EVQLVQSGPELKKPGASVKVSCKASGYTFTNYGMNWVRQAPGQGLEWMGWINTYTGETTYADDFKGRFVFSLDTSVSTAYLQISSLKAEDTAVYYCERGGVNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 2 - protein, 214 aa, Artificial Sequence
  Description of Artificial Sequence Synthetic polypeptide
DIQVTQSPSSLSASVGDRVTITCITSTDIDDDMNWYQQKPGKVPKLLISGGNTLRPGVPSRFSGSGSGTDFTLTISSLQPEDVATYYCLQSDSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 453 aa, Artificial Sequence
  Description of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  Description of Artificial Sequence Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 6 aa, Artificial Sequence
  Description of Artificial Sequence Synthetic 6xHis tag
HHHHHH

(19) United States
(12) Patent Application Publication
(10) Pub. No.: US 2019/0010499 A1
(43) Pub. Date: Jan. 10, 2019

(54) METHODS FOR IMPROVED APTAMER SELECTION

(71) Applicant: Vitrisa Therapeutics, Inc., Larkspur, CA (US)

(72) Inventors: Carl Erickson, Corte Madera, CA (US); Christopher P. Rusconi, Durham, NC (US)

(21) Appl. No.: 16/037,961

(22) Filed: Jul. 17, 2018

Related U.S. Application Data
(63) Continuation of application No. PCT/US2017/014459, filed on Jan. 20, 2017.
(60) Provisional application No. 62/281,092, filed on Jan. 20, 2016.
(60) Provisional application No. 62/286,220, filed on Jan. 22, 2016.
(60) Provisional application No. 62/297,095, filed on Feb. 18, 2016.

(51) Int. Cl. C12N 15/115; C12Q 1/6811; C12N 15/10

(52) U.S. Cl. CPC C12N 15/115; C40B 40/06; C12N 15/1034; C12Q 1/6811

(57) ABSTRACT

Methods are provided herein for generating and selecting aptamers. The aptamers can be suitable as therapeutic aptamers for the treatment of a disease or disorder. The methods provided may improve the efficiency and/or effectiveness of generating a therapeutic aptamer over traditional methods. The methods may generally involve selective pressure including competitive elution to generate aptamers that specifically bind to therapeutically-relevant epitopes and have a desired mechanism of action.

### CROSS-REFERENCE

This application is a continuation application of International Patent Application No. PCT/US2017/014459, filed Jan. 20, 2017, which claims the benefit of U.S. Provisional Application Nos. 62/281,092, filed Jan. 20, 2016; 62/286,220 filed Jan. 22, 2016; and 62/297,095, filed Feb. 18, 2016, which applications are incorporated herein by reference.

### SEQUENCE LISTING

The instant application contains a Sequence Listing which has been submitted electronically in ASCII format and is hereby incorporated by reference in its entirety. Said ASCII copy, created on Jan. 19, 2017, is named 49644-703 601 SL.txt and is 10,529 bytes in size.

### BACKGROUND OF THE INVENTION

Methods are currently used for the generation of aptamers. These methods may select for aptamers that bind to a target molecule. However, these methods may not enable generation of aptamers with binding properties specific to a priori specified epitopes on the target molecule. Oftentimes, these aptamers have no activity or undesired activities and these methods may not be the most effective or efficient at generating aptamers that are suitable as therapeutic agents. The disclosure herein provides methods for the generation of therapeutic aptamers that target specific therapeutically-relevant epitopes (e.g., bioactive epitopes).

### SUMMARY OF THE INVENTION

In one aspect, a method for generating an aptamer is provided. The method involves generating an aptamer library; incubating the aptamer library with a target molecule, wherein at least one aptamer of the aptamer library binds to the target molecule to form an aptamer-target molecule complex; incubating the aptamer-target molecule complex with a competitor that is capable of binding to the target molecule, wherein when the competitor binds to the target molecule, the aptamer of the aptamer-target molecule complex is released; and recovering the released aptamer. The method may further involve sequencing the released aptamer. The method may further involve optimizing the released aptamer. As non-limiting examples, optimization can result in increasing the affinity or activity of the aptamer. The released aptamer may have similar binding properties to the target molecule as compared with the binding properties between the competitor and the target molecule. For example, the released aptamer may bind to a similar epitope of the target molecule as compared with the competitor. For example, the released aptamer may bind to a similar epitope of the target molecule and at a similar affinity as compared with the competitor. For example, the released aptamer may bind to a similar epitope of the target molecule and have a similar biological effect against the target molecule as compared with the competitor.

The method may further involve introducing the aptamer library to an immobilization field and discarding aptamers from the aptamer library that do not bind to said immobilization field. The method may further involve introducing the aptamer-target molecule complex to an immobilization field and retaining the aptamer-target molecule complex when it binds to said immobilization field. These steps in the method can be carried out once or more than once. The immobilization field may include a column, a well plate, or a bead, including a coated magnetic bead.

With respect to the described methods, the competitor may be capable of binding to the target molecule at a pre-determined position. For example, the pre-determined position may be an exosite, or a catalytic cleft. With respect to the described methods, the competitor may be capable of binding to the target molecule at a pre-determined position. For example, the pre-determined position may be an exosite, or a catalytic cleft. In another example, the pre-determined position may be the portion of a cytokine or growth factor that binds to the cognate receptor(s) of said cytokine or growth factor. Alternatively, the pre-determined position may be the portion of a receptor that binds to the cognate cytokine or growth factor which signals via the receptor. The pre-determined position may be a portion of a cytokine, a growth factor or a receptor for a cytokine or growth factor that, when the cytokine or growth factor is in complex with its cognate receptor, is responsible for binding to an accessory protein necessary for the cytokine or growth factor-receptor complex to generate a complex capable of transducing a signal. The method may further involve incubating the aptamer-target molecule complex with more than one concentration of the competitor. The competitor may be an antibody. The antibody may be a polyclonal or a monoclonal antibody. The competitor may be an antibody fragment, such as a Fab. The competitor may be a single-chain antibody. The method may further involve testing the released aptamer for a function associated with the target molecule. The function associated with the target molecule may be related to the specificity of the released aptamer for the target molecule. The function associated with the target molecule may be related to the affinity of the released aptamer for the target molecule. The function associated with the target molecule may be related to a biological function associated with the target molecule, wherein the released aptamer may either reduce or enhance the biological function. Without limitation, the target molecule may include a protein, a nucleic acid, or a lipid.

Further, the above-mentioned aptamer library may be a RNA aptamer library, a modified RNA aptamer library, a DNA aptamer library, or a modified DNA aptamer library.

In another aspect, the target molecule may include a protein that is expressed during an innate immune response, or during an adaptive immune response, or during an autoimmune response. The protein may be expressed when a cancer or tumor is present in a subject. The protein may be expressed as part of a complement pathway or an alternative complement pathway. In certain embodiments, the protein may be Factor D or Factor P. Moreover, the competitor may be an antibody such as an anti-Factor D Fab having an amino acid sequence of heavy chain variable region according to SEQ ID NO: 1 and an amino acid sequence of light chain variable region according to SEQ ID NO:2; or MAb 166-32 or LS-C135735.

In another aspect, an aptamer produced by any of the described methods is disclosed. Such aptamer may be used for treating a subject having an ocular disease. Without limitation, the ocular disease may be macular degeneration, age-related macular degeneration, dry age-related macular degeneration, or geographic atrophy.

In one aspect, a method is provided for selecting a desired aptamer with high affinity for a target epitope of a target molecule, the method comprising: (a) obtaining an aptamer library comprising a plurality of aptamers; (b) incubating the aptamer library with an isolated target molecule comprising the target epitope to form an aptamer-target molecule complex by binding of at least one aptamer of the aptamer library to the target epitope; (c) incubating the aptamer-target molecule complex with a competitor capable of specifically binding to the target epitope, thereby eluting the at least one aptamer from the target epitope; and (d) recovering the at least one aptamer. In some cases, the at least one aptamer binds to the target epitope with a K_{d} of less than about 100 nM. In some cases, the method further comprises prior to (b), immobilizing the target molecule to a solid support. In some cases, the method further comprises, prior to (b), contacting the aptamer library with the solid support in the absence of the target molecule to remove non-specific aptamers. In some cases, the competitor has a higher affinity for the target epitope than the at least one aptamer. In some cases, (c) further comprises providing the competitor at a high molar excess relative to the aptamer:target molecule complex. In some cases, (c) further comprises incubating the competitor with the aptamer:target molecule complex at a ratio of at least 1000:1. In some cases, (c) further comprises incubating the competitor with the aptamer:target molecule complex for 2 hours or less. In some cases, the method further comprises, prior to (b), depleting non-target epitope binding aptamers from the aptamer library, comprising: (i) incubating the target molecule with the competitor such that the competitor binds to the target epitope on the target molecule to generate a competitor:target molecule complex; (ii) incubating the competitor:target molecule complex with the aptamer library such that non-target epitope binding aptamers bind to the competitor:target molecule complex and target epitope-binding aptamers do not bind to the competitor:target molecule complex; and iii) collecting the target epitope-binding aptamers, thereby depleting non-target epitope binding aptamers from the aptamer library.

In another aspect, a method is provided for selecting a desired aptamer with high affinity for a target epitope of a target molecule, the method comprising: (a) obtaining an aptamer library comprising a plurality of aptamers; (b) incubating the aptamer library with an isolated target molecule comprising the target epitope to form an aptamer-target molecule complex by binding of at least one aptamer of the aptamer library to the target epitope; (c) incubating the aptamer-target molecule complex with a competitor capable of specifically binding to the target epitope, thereby eluting the at least one aptamer from the target epitope; (d) recovering the at least one aptamer; and (e) iteratively repeating (b)-(d) one or more times, thereby selecting for a desired aptamer with high affinity for a target epitope of a target molecule, wherein the at least one aptamer binds to the target epitope with a K_{d} of less than about 100 nM. In some cases, the method further comprises prior to (b), immobilizing the target molecule to a solid support. In some cases, the method further comprises, prior to (b), contacting the aptamer library with the solid support in the absence of the target molecule to remove non-specific aptamers. In some cases, the competitor has a higher affinity for the target epitope than the at least one aptamer. In some cases, (c) further comprises providing the competitor at a high molar excess relative to the aptamer:target molecule complex. In some cases, (c) further comprises incubating the competitor with the aptamer:target molecule complex at a ratio of at least 1000:1. In some cases, (c) further comprises incubating the competitor with the aptamer:target molecule complex for 2 hours or less. In some cases, (e) further comprises, iteratively repeating (b)-(d) one or more times, each time with a successively greater amount of competitor in (c). In some cases, the method further comprises, prior to (b), depleting non-target epitope binding aptamers from the aptamer library, comprising: (i) incubating the target molecule with the competitor such that the competitor binds to the target epitope on the target molecule to generate a competitor:target molecule complex; (ii) incubating the competitor:target molecule complex with the aptamer library such that non-target epitope binding aptamers bind to the competitor:target molecule complex and target epitope-binding aptamers do not bind to the competitor:target molecule complex; and iii) collecting the target epitope-binding aptamers, thereby depleting non-target epitope binding aptamers from the aptamer library.

In another aspect, a method is provided for selecting a desired aptamer that specifically binds to a target epitope with high affinity, the method comprising: (a) obtaining an aptamer library; (b) incubating the aptamer library with a target molecule comprising the target epitope to form an aptamer-target molecule complex by at least one aptamer binding to the target epitope; (c) incubating a competitor, capable of specifically binding to the target epitope, with the aptamer-target molecule complex at a ratio of at least 1000:1 for about 2 hours or greater, thereby eluting the at least one aptamer from the target epitope; and recovering the at least one aptamer, thereby selecting for a desired aptamer that specifically binds to a target epitope with high affinity. In some cases, the method further comprises, prior to (b), immobilizing the target molecule to a solid support. In some cases, the method further comprises, prior to (b), contacting the aptamer library with the solid support in the absence of the target molecule to remove non-specific aptamers. In some cases, (c) further comprises providing the competitor at a high molar excess relative to the aptamer:target molecule complex. In some cases, the method further comprises performing two or more iterative rounds of (c). In some cases, the method further comprises, repeating (a)-(d) one or more times, each time with a successively greater amount of competitor in (c). In some cases, the method further comprises, prior to (b), depleting non-target epitope binding aptamers from the aptamer library, comprising: (i) incubating the target molecule with the competitor such that the competitor binds to the target epitope on the target molecule to generate a competitor:target molecule complex; (ii) incubating the competitor:target molecule complex with the aptamer library such that non-target epitope binding aptamers bind to the competitor:target molecule complex and target epitope-binding aptamers do not bind to the competitor:target molecule complex; and (iii) collecting the target epitope-binding aptamers, thereby depleting non-target epitope binding aptamers from the aptamer library. In some cases, the method further comprises repeating (c) one or more times in an iterative fashion. In some cases, the repeating comprises incubating the aptamer-target molecule complex with successively greater amounts of competitor. In some cases, the repeating comprises incubating the aptamer-target molecule complex with different competitors.

In another aspect, a method is provided for selecting a desired aptamer, the method comprising: (a) obtaining an aptamer library; (b) incubating a target molecule comprising a target epitope with a competitor capable of specifically binding to the target molecule at the target epitope, thereby generating at least one competitor:target molecule complex; (c) incubating the at least one competitor:target molecule complex with the aptamer library such that non-target epitope binding aptamers bind to the at least one competitor:target molecule complex and target epitope-binding aptamers do not bind to the at least one competitor:target molecule complex; (d) collecting the target epitope-binding aptamers, thereby depleting non-target epitope binding aptamers from the aptamer library; (e) incubating the depleted aptamer library with the target molecule, wherein at least one target-epitope binding aptamer of the depleted aptamer library specifically binds to a target molecule to form an aptamer-target molecule complex; (0 incubating the aptamer-target molecule complex with the competitor, wherein the competitor competes with the at least one aptamer for the target epitope thereby eluting the at least one aptamer from the target epitope; and (g) recovering the at least one aptamer, thereby selecting for a desired aptamer.

In another aspect, a method is provided for depleting an aptamer library of non-specific aptamers, the method comprising: (a) obtaining an aptamer library comprising a plurality of aptamers; (b) contacting the aptamer library with a solid support, wherein the solid support is to be used in successive rounds of selection; (c) collecting any aptamers of the aptamer library that do not bind to the solid support, thereby depleting the aptamer library of non-specific aptamers; (d) incubating the depleted aptamer library with a target molecule comprising a target epitope, wherein the target molecule is provided at a higher copy number than a sequence copy number of the aptamer library; and (e) recovering any aptamers that bind to the target molecule, thereby generating a first enriched pool of aptamers. In some cases, the method further comprises: (i) incubating the target molecule with a competitor capable of specifically binding to the target molecule at the target epitope, thereby generating a competitor:target molecule complex; (ii) incubating the competitor:target molecule complex with the first enriched pool of aptamers such that non-target epitope binding aptamers bind to the competitor:target molecule complex and target epitope-binding aptamers do not bind to the competitor:target molecule complex; and (iii) collecting the target epitope-binding aptamers, thereby generating a second enriched aptamer pool depleted of the non-target epitope-binding aptamers. In some cases, the method further comprises: (iv) incubating the second enriched aptamer pool with the target molecule, wherein at least one target-epitope binding aptamer of the second enriched aptamer pool specifically binds to the target molecule to form an aptamer-target molecule complex; (v) incubating the aptamer-target molecule complex with the competitor, wherein the competitor competes with the at least one aptamer for the target epitope thereby eluting the at least one aptamer from the target epitope; and (vi) recovering the at least one aptamer, thereby selecting for an aptamer.

In another aspect, a method is provided for selecting for a desired aptamer that specifically binds to a target epitope on a target molecule with high affinity, the method comprising: (a0 incubating target molecules comprising the target epitope with a non-competitive binder that blocks some, but not all, of the target epitopes on the target molecules; (b) obtaining an aptamer library comprising a number of aptamers capable of binding to the target epitope, wherein the number of aptamers capable of binding to the target epitope is greater than the number of unblocked epitopes and the number of aptamers have different binding affinities for the target epitope; (c) incubating the target molecules comprising blocked and unblocked target epitopes with the aptamer library such that the number of aptamers compete for binding to an unblocked target epitope, wherein an aptamer of the number of aptamers with higher affinity for the target epitope binds the target epitope and an aptamer of the number of aptamers with lower affinity for the target epitope does not bind the target epitope, thereby selecting for a desired aptamer that specifically binds to a target epitope with high affinity. In some cases, the non-competitive binder comprises an anionic polymer. In some cases, the anionic polymer is tRNA, dextran sulfate, heparin, or hyaluronic acid.

In any one of the preceding methods, the competitor may be an antibody or antibody fragment thereof, a small molecule or a peptide. In any one of the preceding methods, the antibody may be a monoclonal antibody. In any one of the preceding methods, the desired aptamer has a desired mechanism of action. In any one of the preceding methods, the desired aptamer alters a biological function of the target molecule. In any one of the preceding methods, the desired aptamer inhibits a biological function of the target molecule. In any one of the preceding methods, the desired aptamer enhances a biological function of the target molecule. In any one of the preceding methods, therapeutic aptamer binds to the target molecule with a K_{d} of about 100 nM or less. In any one of the preceding methods, the therapeutic aptamer binds to the target molecule with a K_{d} of about 50 nM or less. In any one of the preceding methods, the therapeutic aptamer binds to the target molecule with a K_{d} of about 10 nM or less. In any one of the preceding methods, the therapeutic aptamer binds to the target molecule with a K_{d} of about 5 nM or less. In any one of the preceding methods, the target molecule is a recombinant protein. In any one of the preceding methods, the competitor is a therapeutic molecule utilized for the treatment of a target disease. In any one of the preceding methods, the aptamer library comprises at least 10¹⁴ different aptamer sequences. In any one of the preceding methods, the competitor is incubated with the aptamer-target molecule complex at a high molar excess. In any one of the preceding methods, the competitor is incubated with the aptamer-target molecule complex at a ratio of at least 1000:1. In any one of the preceding methods, the target molecule is a recombinant protein or peptide. In any one of the preceding methods, the aptamer library is a DNA aptamer library, a modified DNA aptamer library, an RNA aptamer library, or a modified RNA aptamer library. In any one of the preceding methods, the methods further comprise amplifying the desired aptamer. In any one of the preceding methods, the methods further comprise sequencing the desired aptamer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a non-limiting example of a method workflow according to an embodiment of the disclosure.

FIG. 2 depicts a non-limiting example of a method workflow according to an embodiment of the disclosure.

FIG. 3 depicts a non-limiting example of a method workflow according to an embodiment of the disclosure.

FIG. 4 depicts a non-limiting example of a method workflow according to an embodiment of the disclosure.

FIG. 5 depicts a non-limiting example of modified library aptamers suitable to perform the methods described herein.

FIG. 6 depicts a non-limiting example of quantitative next-generation sequencing analysis at various stages of the methods described herein.

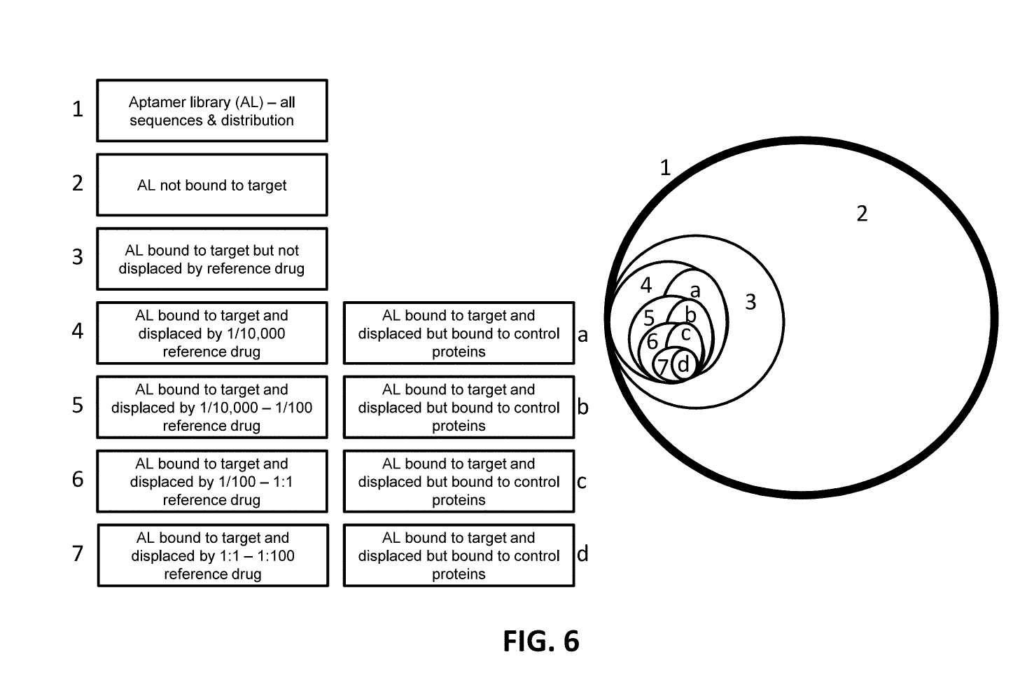

FIG. 7 depicts a non-limiting example of a positive selection step in accordance with an embodiment of the disclosure.

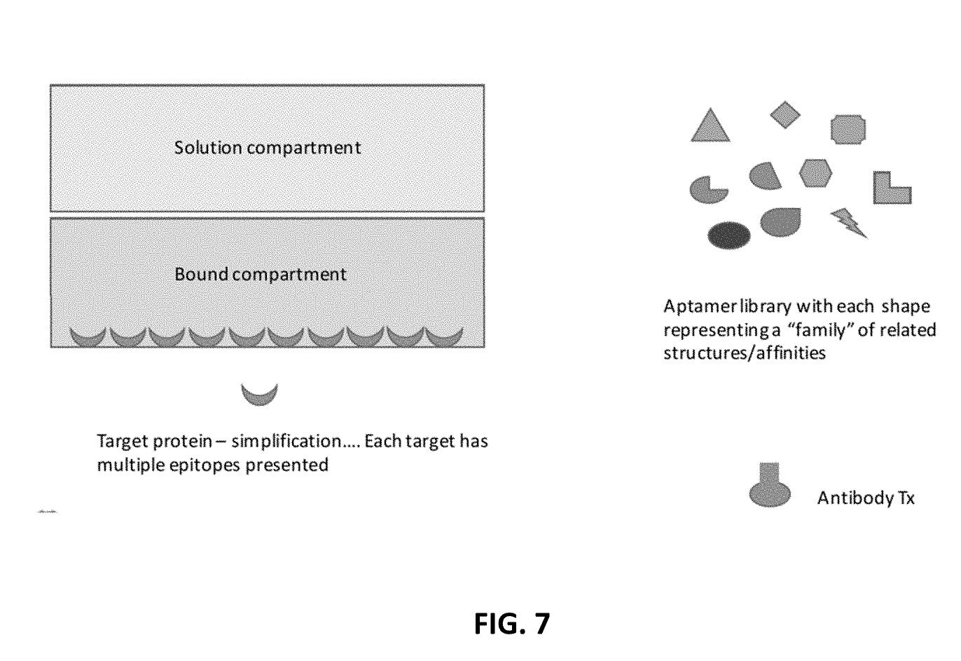

FIG. 8 depicts a non-limiting example of a positive selection step in accordance to an embodiment of the disclosure.

FIG. 9 depicts a non-limiting example of a positive selection step in accordance with an embodiment of the disclosure provided herein.

FIG. 10 depicts a non-limiting example of a positive selection step in accordance with an embodiment of the disclosure.

FIG. 11 depicts a non-limiting example of a positive selection step in accordance with an embodiment of the disclosure.

FIG. 12 depicts a non-limiting example of a positive selection step in accordance with an embodiment of the disclosure.

FIG. 13 depicts a non-limiting example of a counter-selection step in accordance with an embodiment of the disclosure.

FIG. 14 depicts a non-limiting example of a competitive elution step in accordance with an embodiment of the disclosure.

FIG. 15 depicts a non-limiting example of altering the conditions of a positive selection step to select for high affinity binding aptamers in accordance with an embodiment of the disclosure.

FIG. 16 depicts a non-limiting example of altering the conditions of a positive selection step to select for high affinity binding aptamers in accordance with an embodiment of the disclosure.

FIG. 17 depicts enrichment of aptamers to the receptor-binding domain of VEGF₁₆₅ by antibody elution.

FIG. 18 depicts enrichment of aptamers to the receptor-binding domain of VEGF₁₂₁ by antibody elution.

FIG. 19 depicts enrichment of aptamers to the exosite of factor D by antibody elution.

FIG. 20 depicts de-enrichment of aptamers to the exosite of factor D by epitope masking.

FIG. 21 depicts a non-limiting example of a flow-cytometry based analysis of a selection process as described herein.

FIG. 22 depicts a non-limiting example of a method workflow according to an embodiment of the disclosure.

FIG. 23 depicts a plot of the median relative enrichment with 95% confidence intervals of the top 50 most enriched aptamers utilizing a method as disclosed herein.

FIG. 24 depicts individual enrichment plots of the top 25 most enriched aptamers utilizing a method as disclosed herein.

FIG. 25 depicts enrichment plots of aptamers enriched utilizing a method as disclosed herein.

FIG. 26 depicts association curves of various compounds with their respective targets.

FIG. 27 depicts dissociation curves of various compounds with their respective targets.

FIG. 28 depicts a non-limiting example of antibody elution modeling according to an embodiment of the disclosure.

FIG. 29 depicts a non-limiting example of antibody elution modeling according to an embodiment of the disclosure.

FIG. 30 depicts a non-limiting example of antibody elution modeling according to an embodiment of the disclosure.

FIG. 31 depicts a non-limiting example of antibody elution modeling according to an embodiment of the disclosure.

### DETAILED DESCRIPTION OF THE INVENTION

This disclosure provides methods of enriching an aptamer library in order to generate therapeutic aptamers that bind a specific epitope of a target molecule (e.g., protein) with high affinity. The methods herein provide solutions to the presence of non-epitope specific aptamers or low affinity target-epitope binding aptamers present in a library or aptamer set. They further address the issue of variable aptamer off-rate from the target. Finally, the methods herein allow for a fuller elution of desired aptamers with competitors that bind to therapeutically-relevant epitopes on target molecules and prevention of re-binding of aptamers to the target epitope using said competitors to afford maximal enrichment. The methods disclosed herein may generally be used to generate therapeutic aptamers that specifically bind to therapeutically-relevant epitopes of target molecules.

Generally, the methods involve obtaining an enriched aptamer library, for example, an aptamer library that has been enriched for aptamers that specifically bind to a target molecule. The methods further involve subjecting the enriched aptamer library to one or more rounds of selective pressure to further bias the library for aptamers that bind to a target epitope on the target molecule with high affinity. Selective pressure may refer to any method described herein to bias the aptamer library for target epitope-binding aptamers. Various methods of exerting selective pressure on an aptamer library are described herein and it is to be understood that any combination and order of selective pressure methods may be utilized to enrich an aptamer library. Non-limiting examples of selective pressure, which are described in greater detail herein, include competitively displacing aptamers from the target epitope with a competitor, counter selection methods that utilize a competitor to block or mask the target epitope, using limited concentrations of target molecule to enrich for higher affinity aptamers, and/or adjusting the stringency of the incubation conditions during selection rounds to favor higher affinity aptamers. Aptamer pools may be amplified and sequenced to identify aptamer sequences present in the pool. The sequencing data may then be subjected to one or more bioinformatics steps involving determining the enrichment of aptamer sequences in a final aptamer pool and comparing the rate of enrichment of aptamer sequences subjected to selective pressure to identify those aptamers that are enriched in response to selective pressure. Aptamers that exhibit high levels of enrichment and/or high rates of enrichment in response to selective pressure may be tested for biological function in functional assays.

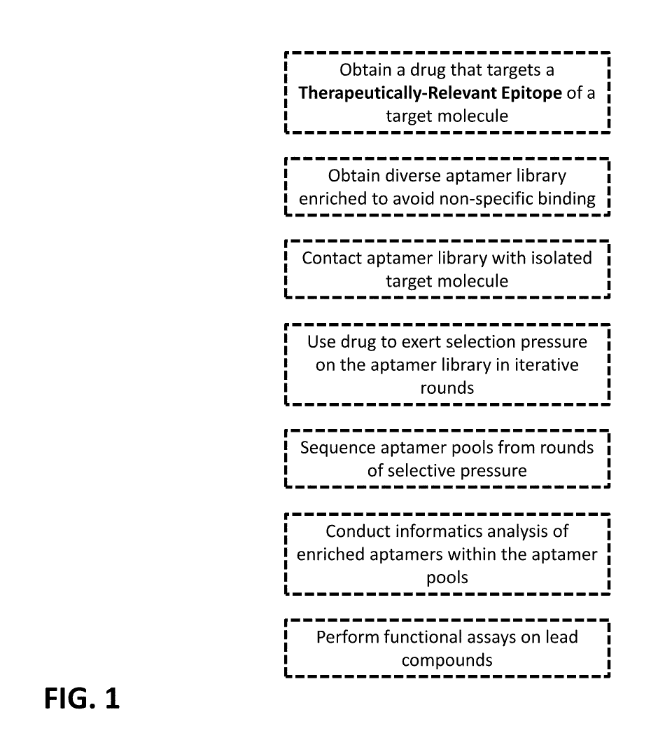

As described in FIG. 1, clinically-relevant drugs may be used to exert selection pressure on a library of aptamers, such that clinically-relevant aptamers are enriched within a library. The clinically-relevant drugs may include any molecules that can modulate the biological function of a target molecule. Often, the biological function of the target molecule may be implicated in a specific disease such that modulation (e.g., inhibition) of the biological function of the target molecule may have a therapeutic effect (e.g., may treat or cure a disease). Generally, the clinically-relevant drug may exert an effect on the target molecule by binding to a specific site on the target molecule, for example, a therapeutically-relevant epitope. These clinically-relevant drugs may be used to selectively compete with aptamers that bind to the target molecule at therapeutically-relevant epitopes. Such aptamers may mimic the therapeutic effects of the clinically-relevant drug, including possessing a mechanism of action similar to the clinically-relevant drug, thus providing therapeutic aptamers with clinical significance.

Often, the methods involve bioinformatics approaches to identify aptamers that are enriched in response to selective pressure with a clinically-relevant drug. Aptamer sequences present in a final aptamer pool (e.g., after performing one or more rounds of selection as described herein) may be compared to a starting aptamer pool (e.g., prior to performing one or more rounds of selection as described herein) and those aptamers that are enriched in the final pool relative to the starting pool may be identified. Further, the relative rate of enrichment of these aptamer sequences can be assessed by comparing the enrichment of these aptamer sequences in pools that are not subjected to selective pressure versus those pools that are. Those aptamers that are enriched in the final pool and demonstrate a high rate of enrichment under selective pressure may be identified as candidate therapeutic aptamers. After candidate therapeutic aptamers are identified, these aptamers may be tested in functional assays that assess the bioactivity and the clinical relevance of these aptamers.

The aptamer selection methods herein involve various enrichment, selection, and isolation steps to arrive at a final set of aptamer(s) of interest. These steps can include one or more “negative selection” steps in which aptamers that are not of interest are removed from the library. Examples of negative selection steps include removal of aptamers that non-specifically bind a control substrate such as magnetic beads. These steps can also include one or more “positive selection” steps in which aptamers that bind the target molecule (or target epitope) are selected or isolated from other aptamers. These steps can also include one or more “counter-selection” steps in which a ligand that is known to bind a target epitope is allowed to bind to the epitope and prevents aptamers of interest from binding to it to generate aptamer pools for use in subsequent positive selection rounds to isolate aptamers to the desired epitope. These steps can also include one or more “competitive elution” steps in which a known ligand, often with bioactivity, to the epitope (e.g., a bioactive-epitope specific competitor) is used to outcompete an aptamer that was previously bound to the epitope, thereby eluting it off the target. These steps can also include one or more “bioinformatics” steps in which relative enrichment of aptamers during different selection rounds can be analyzed to identify those aptamers that are enriched in response to selective pressure with bioactive-epitope specific competitor elution (or depletion if using a de-enrichment process).

The above steps can be performed in various order and iterations. Specific concentrations of target, ligands, and aptamers, as well as conditions and timing of elution are also contemplated and discussed further below.

In general, a “target molecule” as used herein refers to a biological molecule such as a protein, a lipid, a nucleic acid, a cell, and the like. A “target molecule” may include a molecule that is known to have a biological function, and in some cases, is a molecule that has a biological function in a disease or disorder. The “known ligand” (sometimes referred to herein as a “competitor”) that is used in the assays herein is generally a molecule that binds to and modulates the activity of a target molecule. Often, the competitor binds to a therapeutically-relevant epitope, for example, an epitope with a known bioactive function or known to play a role in the pathology of a disease. The competitor can be used to exert a selection pressure on an aptamer library such that aptamers that bind to the same or similar region of the target molecule as the competitor are enriched. Generally, the competitor is a clinically-relevant drug (e.g., has therapeutic significance) such that the aptamers that are enriched preferentially have similar therapeutic properties, including a similar mechanism of action, as the competitor. In specific instances, the competitor is a therapeutic molecule that is used or under clinical development to treat or alleviate the symptoms of a disease or disorder.

In one instance, a negative selection step is performed on an aptamer library. Negative selection may involve removing any aptamers from an aptamer library or set of aptamers that non-specifically bind to a control substrate or removing any aptamers that do not bind to a target molecule. Generally, the control substrate is a substrate that will be used in subsequent selection rounds.

In one instance, a positive selection step is performed on an aptamer library. Positive selection may involve contacting an aptamer library or aptamer pool with a target molecule of interest. Generally, the target molecule of interest includes a target epitope, and often, the target epitope is a target of a therapeutic molecule such as the ligand/competitor. Positive selection may enrich an aptamer library or pool for those aptamers that bind to the target molecule, either specifically or non-specifically. Aptamers that specifically bind to the target molecule may bind to the target epitope, or to non-target epitopes of the target molecule. Thus, one or more additional methods may be employed to further enrich the aptamer library or pool for aptamers that specifically bind to the target epitope. After each round of positive selection, aptamers that bind to the target molecule may be collected and amplified to e.g., increase the number of low copy number desired binders present in the aptamer pool.

In some instances, it may be important to ensure that rare epitope-binding aptamers in an aptamer library are recovered after positive selection. In an aptamer library with high diversity (e.g., >10¹⁴ different aptamer sequences), sequence copy number may be low (e.g., 5-15 copies). Thus, in such cases, the target molecule may be provided at a high concentration relative to the copy number of the aptamer library such that the number of available epitopes on the target molecule is not limiting. Often, these methods are performed early in the selection process, for example, in the first, second, third, fourth, and/or fifth rounds of selection to ensure that rare epitope-binding aptamers are not lost.

In one instance, an aptamer library is enriched for target-epitope binding aptamers. In one non-limiting example, counter-selection may be used to enrich for target-epitope binding aptamers. Counter-selection may generally involve contacting a target molecule comprising a target epitope with a competitor that is capable of specifically binding to the target epitope prior to the addition of the aptamer library or pool. The competitor may be provided at a high concentration such that all or substantially all of the target epitopes are blocked or masked. Upon addition of the aptamer library or pool, non-target epitope binding aptamers may bind to the target molecule, however, those aptamers that specifically bind to the target epitope may be prevented from binding. Those aptamers that bind to the target molecule may be segregated from those aptamers that do not bind, and non-binding aptamers may be recovered. In such cases where an aptamer pool enriched for target molecule-binding aptamers is provided, those aptamers that do not bind to the target molecule during counter-selection may preferentially include only those target epitope-binding aptamers. Such collected aptamers may be subjected to additional rounds of positive selection, followed by counter-selection and positive selection, to further enrich for target epitope-binding aptamers.

In another instance, target epitope-binding aptamers may be enriched in an aptamer pool by performing competitive elution. Competitive elution may involve binding a pool of target epitope-binding aptamers to the target epitope, followed by incubation with a competitor that competes with the target-epitope binding aptamer for the target epitope. If the competitor is provided at a high concentration and/or has a higher affinity for the target epitope than the bound aptamer, the bound aptamer may be displaced from the target epitope. Any displaced aptamers may be collected and subjected to additional rounds of selection to enrich for high affinity aptamers.

In one instance, an aptamer pool enriched for target epitope-binding aptamers is further enriched for those aptamers with high affinity. In one example, an aptamer pool (for example, one already enriched for target epitope-binding aptamers), may be contacted with a low concentration of target molecule such that the number of available target epitopes is limited. In such cases, those epitopes with the highest affinity may preferentially bind the target epitope whereas those aptamers with lower affinity may not bind. The higher affinity binders may then be collected and iterative rounds of such selection, or additional selection methods, may be performed to further enrich the pool for high affinity binders. Additionally or alternatively, the target molecule may be contacted with a non-competitive binder (such examples including anionic polymers such as tRNA, dextran sulfate, heparin, hyaluronic acid). In such cases, the non-competitive binder may effectively limit the number of available target epitopes on the target molecule such that high affinity binders preferentially bind.

In some situations, it may be more challenging to recover those aptamers with highest affinity for the target molecule or those aptamers with slow off-rates. In such cases, the kinetics and affinity of the aptamers for the target epitope may need to be considered to ensure maximal recovery of desired aptamers. For example, in competitive elution methods, in order for an aptamer to be outcompeted by the competitor for the target molecule, the aptamer must initially dissociate from the target epitope. For slow off-rate aptamers, the incubation period of competitor with aptamer-target molecule complex may need to be longer to ensure the bound aptamer is able to dissociate. In such cases where the incubation period is not sufficient, slow off-rate aptamers may ultimately be lost. However, aptamers that dissociate may be able to rebind to the target epitope, thereby resulting in lower chances of recovery. Thus, methods may be provided to prevent the aptamer from re-binding to the target epitope after dissociation. The methods herein provide sufficient incubation periods such that slow off-rate aptamers may be maximally recovered and to prevent aptamers from re-binding to the target epitope after dissociation. In non-limiting examples, high concentrations of competitor may be used during competitive elution steps (for example, at least 1000:1 competitor:aptamer), longer incubation periods may be used, and/or higher stringency conditions may be used. In one specific example, two or more iterative rounds of competitive elution may be used, and the aptamers recovered in each round may be pooled. In some cases, each round of competitive elution may be about 60 minutes or more, for example, about 60 minutes, about 90 minutes, about 120 minutes, about 180 minutes, or more. With each successive round of competitive elution, the same competitor and conditions may be used in each round, or in some cases, the concentration of competitor may be increased or a different competitor with a higher affinity for the target epitope may be used to increase the stringency of the elution process.

The methods may further include performing one or more bioinformatics steps. For example, samples of aptamer pools at each (or a few) round(s) of selection may be collected and used to assess the identity of aptamers present in the pool. Relative enrichment and rates of enrichment may be compared amongst the various aptamer pools sampled at different rounds during the selection process to identify those aptamers that show the highest relative enrichment and/or highest rate of enrichment in response to selection pressure. Often, an aptamer library enriched by positive selection or prior to performing one or more rounds of selection pressure as described herein may serve as the parental round against which subsequent rounds, generally involving selective pressure, are compared. For example, the relative enrichment of aptamer pools after performing one or more rounds of competitive elution may be compared with the parental round (e.g., a starting pool prior to selective pressure) to identify those aptamers that are enriched in response to selection pressure with a bioactive epitope specific competitor. Enrichment may be calculated by first calculating the enrichment of aptamers in the final aptamer pool after the final round of selection. Enrichment for a given aptamer may generally be calculated as the fraction of the aptamer sequence identified in the final aptamer pool divided by the fraction of the aptamer sequence in the library used as the starting point for the selection process. Aptamer sequences with enrichment greater than 10-100× may define a query set of sequences enriched in response to selective pressure of bioactive epitope competitor elution (or depletion if using a de-enrichment process). The query set may then be used to compare the rate of enrichment from the selective pressure rounds versus the positive selection rounds and to control fractions. For example, those aptamers that are enriched more rapidly in response to selection pressure may be of interest. Those sequences of interest may then be screened in functional assays to assess the biological function.

These methods may be suitable for the identification of rare target epitope-binding aptamers that might normally be discarded during positive selection methods. For example, positive selection methods may preferentially enrich for those aptamers that are the most frequent in an aptamer pool, however, there may be desired aptamers in a pool that are present at a low frequency. The methods provided herein include identifying those aptamers that have a low frequency in an aptamer pool, and analyzing enrichment of these aptamers in subsequent selection rounds. An aptamer present in very low starting amounts may still be present in low amounts, even after multiple rounds of selection. However, the methods herein provide for identifying desired aptamers by assessing the rate of enrichment during one or more rounds of selective pressure (e.g., competitive elution). Although these aptamers may still be present at very low frequency after selective pressure, there may be an increased rate of enrichment that can be observed in response to the selective pressure, thereby suggesting that these aptamers may be therapeutically relevant.

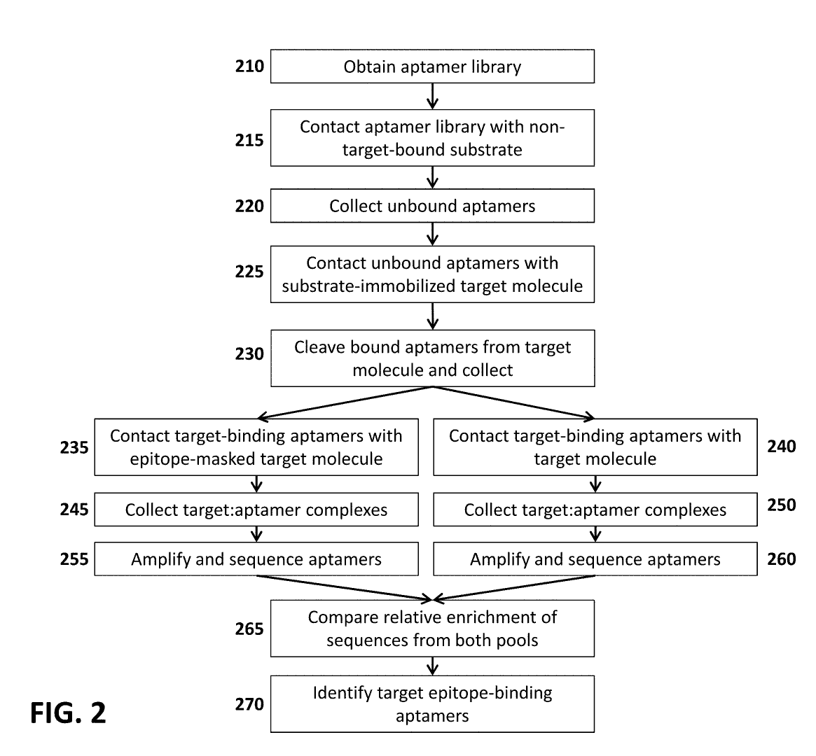

In one instance, a method as described in FIG. 2 is provided for the selection of high affinity target-epitope binding aptamers. In general, the method may begin by obtaining an aptamer library 210 that may be used in aptamer selection methods provided herein. In some cases, aptamers with non-specific binding properties may be removed from the aptamer library, such as by contacting the aptamer library with a solid support (e.g., a bead) or partitioning matrix (e.g., nitrocellulose) that lacks the target molecule. Generally, the solid support or partitioning matrix may be the same or similar as that to be used in subsequent selection rounds. Such negative selection processes 215 may be performed at any step of the selection process; preferably, such negative selection is performed prior to initiating positive selection. Aptamers that remain unbound may be collected 220 and used for further rounds of selection as described below.

The methods may further include any number of positive selection methods involving contacting the unbound aptamer pool with a target molecule 225. In some cases, the target molecule may be immobilized to a substrate (e.g., a bead) or partitioning matrix 225. Such positive selection methods may be performed after the performance of negative selection or in the absence of negative selection. During positive selection, aptamers with affinity for the target molecule may bind to the target molecules, including to desirable epitopes and to undesirable epitopes. In cases where target molecules are attached to a solid support such as a bead, non-target binders may be preferentially present in solution. In some cases, the copy number of target molecules present during any positive selection step may be higher, in some cases much higher, than the copy number of individual aptamers in the aptamer library such that every aptamer that has the ability to bind to the target molecule binds. The high target:aptamer copy number ratio may enable capture of rare aptamers that may otherwise fail to bind the target molecule. Target-binding aptamers may then be cleaved or otherwise removed from the target molecule and collected 230 for further selection methods as described below.

The method may further include de-enriching a target-binding aptamer pool for target epitope-specific aptamers. Such methods may involve one or more counter-selection methods. Such counter-selection may involve pre-binding the target molecule with a competitor molecule. Generally, the competitor molecule is designed to target a specific epitope in the target molecule, thereby blocking or masking the target epitope. The competitor molecule may, in some instances, be a clinically-relevant drug such as a monoclonal antibody, a small molecule, or a peptide. The method may further include introducing the pre-bound target molecule to the enriched pool of target-binding aptamers 235. In such counter-selection methods, target epitope-binding aptamers may be unable to bind to the target molecule (because the specific epitope is blocked) whereas non-target epitope-binding aptamers may be able to bind the target molecule. In cases where the target molecule (or competitor molecule) is attached to a solid support, aptamers that bind to the specific epitope may likely remain in solution while non-epitope specific aptamers that bind to the target molecule (or competitor molecule) may remain bound to the solid support. Target molecule:aptamer complexes may be collected 245, 250 and the aptamers may be amplified and sequenced 255, 260. Aptamers that bind the target epitope may be identified by, e.g., comparative sequencing analysis, such as by comparing the sequences of aptamers that bind to the target molecule in the absence of competitor with those aptamers that bind to the target molecule in the presence of competitor 265. In such cases, those aptamers that bind the target epitope may be more frequent in the absence of competitor, or decreased in the presence of competitor 270. Bioinformatics approaches may be utilized to assess the relative enrichment and rate of enrichment of aptamers in the final pool. In this example, those aptamers that are therapeutically relevant may be depleted in the final pool as compared to the starting pool. For example, the therapeutically relevant aptamers may be less enriched in the final pool. Additionally, therapeutically relevant aptamers may demonstrate a higher rate of depletion from the starting pool to the final pool when subjected to selective pressure.

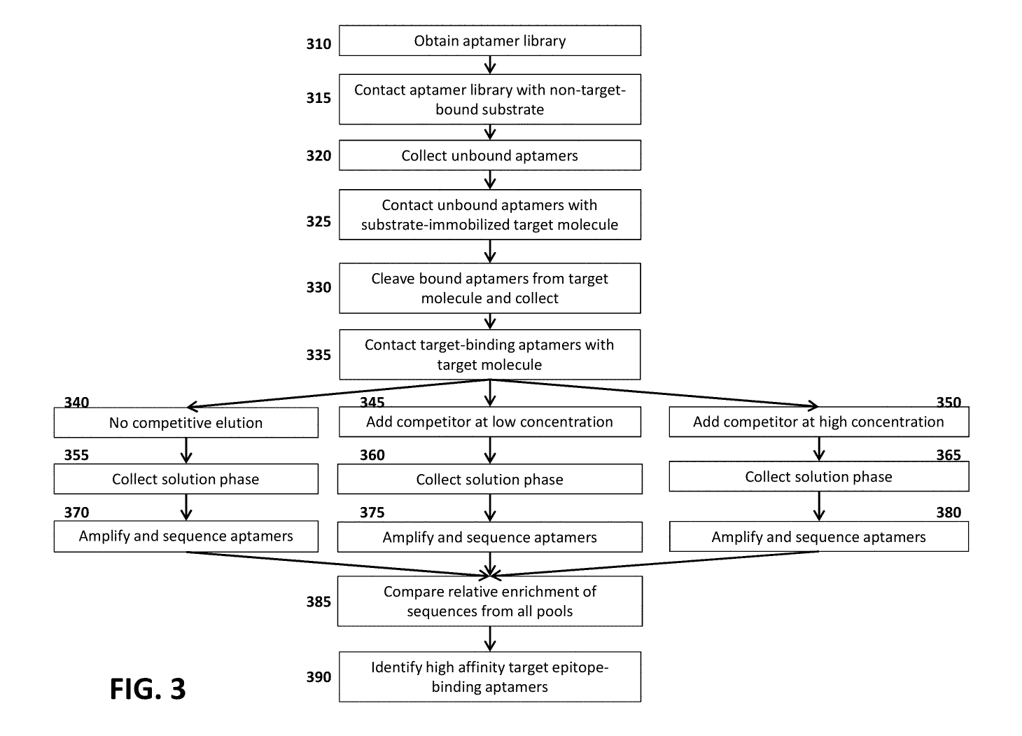

In another instance, a method as described in FIG. 3 is provided for the selection of high affinity target-epitope binding aptamers. In general, the method may begin by obtaining an aptamer library 310 that may be used in aptamer selection methods provided herein. In some cases, aptamers with non-specific binding properties may be removed from the aptamer library, such as by contacting the aptamer library with a solid support (e.g., a bead) or partitioning matrix (e.g., nitrocellulose) that lacks the target molecule. Generally, the solid support or partitioning matrix may be the same or similar to that to be used in subsequent selection rounds. Such negative selection process 315 may be performed at any step of the selection process; preferably, such negative selection is performed prior to initiating selection. Aptamers that remain unbound may be collected 320 and used for further rounds of selection as described below.

The methods may further include any number of positive selection methods involving contacting the unbound aptamer pool with a target molecule 325. In some cases, the target molecule may be immobilized to a substrate (e.g., a bead) or partitioning matrix 325. Such positive selection methods may be performed after the performance of negative selection or in the absence of negative selection. During positive selection, aptamers with affinity for the target molecule may bind to the target molecules, including to desirable epitopes and to undesirable epitopes. In cases where target molecules are attached to a solid support such as a bead, non-target binders may be preferentially present in solution. In some cases, the copy number of target molecules present during any positive selection step generally may be higher, in some cases much higher, than the copy number of individual aptamers in the aptamer library such that every aptamer that has the ability to bind to the target molecule binds. The high target:aptamer copy number ratio may enable capture of rare aptamers that may otherwise fail to bind the target molecule. Target-binding aptamers may then be cleaved or otherwise removed from the target molecule and collected 330 for further selection methods as described below.

The method may further include the positive enrichment of an aptamer pool for epitope-specific aptamers with high affinity. In some cases, such methods may include one or more iterative rounds of competitive elution. In such competitive elution methods, the target molecule may be introduced to an aptamer pool such that target-binding aptamers bind to the target molecule 335. A competitor molecule with affinity for the target epitope may then be added such that the competitor competes with the target epitope-binding aptamers for the target epitope. In such cases, if the competitor molecule has higher affinity for the target epitope than the target-epitope binding aptamers, the target-epitope binding aptamers may be displaced from the target molecule. In one such example, a competitor molecule may be added at a low concentration to one sample 345, at a high concentration to another sample 350, or no competitor may be added 340. Any aptamers that have been displaced from the target molecule may be preferentially found in the solution phase and may be collected, amplified, and sequenced 355, 360, 365, 370, 375, 380. The relative enrichment of aptamer sequences identified from each pool may be compared 385. Aptamers that bind to the target epitope may be preferentially found in those pools in which a competitor was added, and further, aptamers with higher affinity for the target epitope may be preferentially found in the pools in which a high concentration of competitor was added 390. Bioinformatics approaches may be utilized to assess the relative enrichment and/or rate of enrichment of sequences present in the final aptamer pool versus the starting pool. Those aptamers that are enriched and/or demonstrate a higher rate of enrichment in response to selective pressure may be identified as therapeutically relevant aptamers. These aptamers can be tested for biological activity in functional assays.

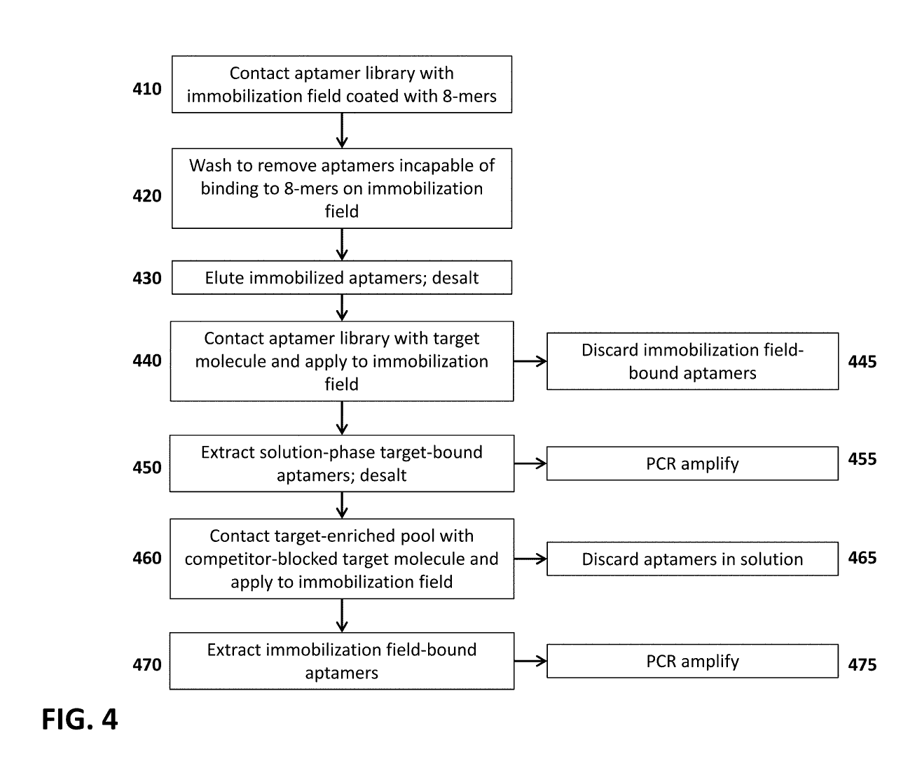

In other instances, methods are provided for aptamer selection using a solution-based target molecule, as described in FIG. 4. An aptamer library may be contacted with a solid substrate 410. The solid substrate may be any solid substrate (e.g., beads) or partitioning matrix (e.g., nitrocellulose) as described herein. In some cases, the surface of the solid substrate is coated with random oligomers (e.g., nucleic acids). The random oligomers may be 6-mers, 7-mers, 8-mers, 9-mers, 10-mers, 11-mers, or 12-mers. The solid substrate may be washed to remove any aptamers that are incapable of binding to the oligomers on the solid substrate 420. The bound aptamers may be eluted from the solid substrate and desalted 430 for further selection processes as described below.

In some cases, the aptamer library may then be contacted with a solution-based target molecule (i.e., not bound to a solid support) 440 and then applied to a solid substrate as described above. In such cases, target-bound aptamers may be blocked from binding to the solid substrate whereas non-target-bound aptamers may bind to the solid substrate. In some cases, the solid substrate-bound aptamers may be discarded 445. The target-bound aptamers may be extracted from the solution phase 450 and amplified to generate a target-bound aptamer pool 455. One or more further iterations of the above methods may be performed.

The target-bound aptamer pool may then be contacted with a competitor-blocked target molecule and then applied to a solid substrate 460. In some cases, the competitor is as described above and may preferentially bind to a target epitope of the target molecule, thereby masking or blocking the target epitope. In such cases, non-target epitope-binding aptamers may bind to the target molecule whereas target epitope-binding aptamers may not bind to the target molecule. In this example, non-target epitope-binding aptamers may be preferentially found in the solution phase (i.e., unable to bind the solid substrate) whereas target epitope-binding aptamers may be preferentially bound to the solid substrate. Any solution-phase, target-bound aptamers may be discarded 465 and the target epitope-binding aptamers may be extracted from the solid substrate 470. The enriched pool of target epitope-binding aptamers may be amplified 475. One or more further iterations of the above methods may be performed, for example, to enrich for higher affinity binders. At this stage, further rounds of selection may be performed as described herein, for example, one or more rounds of competitive elution to further enrich for high affinity target epitope-binding aptamers. Bioinformatics approaches may be used to assess the relative enrichment and/or rate of enrichment in the final aptamer pool as compared to the starting pool. Those aptamers that demonstrate high enrichment and/or high rate of enrichment may be identified as candidate therapeutic aptamers. These aptamers can be tested in functional assays to assess their biological activity.

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

In general, “sequence identity”, as may be referred to herein, refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

The term “aptamer” as used herein refers to oligonucleotide molecules and/or nucleic acid analogues that bind to a target or target molecule (e.g., a protein) with high affinity and specificity through non-Watson-Crick base pairing interactions. Generally, the aptamers described herein are non-naturally occurring oligonucleotides (i.e., synthetically produced) that are isolated and used for the treatment of a disorder or a disease. Aptamers can bind to essentially any target molecule including, without limitation, proteins, oligonucleotides, carbohydrates, lipids, small molecules, and even bacterial cells. Whereas many naturally occurring oligonucleotides, such as mRNA, encode information in their linear base sequences, aptamers can be distinguished from these naturally occurring oligonucleotides in that binding of the aptamer to a target molecule is dependent upon secondary and tertiary structures of the aptamer rather than a conserved linear base sequence and the aptamer generally does not encode information in its linear base sequence. Aptamers may be suitable as therapeutic agents and may be preferable to other therapeutic agents because: 1) aptamers may be fast and economical to produce because aptamers can be developed entirely by in vitro processes; 2) aptamers may have low toxicity and may lack an immunogenic response; 3) aptamers may have high specificity and affinity for their targets; 4) aptamers may have good solubility; 5) aptamers may have tunable pharmacokinetic properties; 6) aptamers may be amenable to site-specific conjugation of PEG and other carriers; and 7) aptamers may be stable at ambient temperatures. Often, aptamers identified by performing the methods described herein may be “therapeutic” or “therapeutically-relevant” aptamers. These aptamers may specifically bind to a target epitope of a target molecule (e.g., a bioactive epitope) to modulate a biological function of the target molecule.

The term “target” or “target molecule” as used herein refers to any molecule in which a therapeutic aptamer is generated or selected to bind to. The target molecule can be a protein, a peptide, a nucleic acid, a lipid, a small molecule, a biological cell (e.g., a bacterial cell) and the like. In some cases, the target molecule is a protein. In some cases, the target molecule is a protein expressed during an innate immune response. In some cases, the target molecule is a protein expressed during an adaptive immune response. In some cases, the target molecule is a protein expressed during an autoimmune response. In some cases, the target molecule is a protein that is expressed as part of the complement pathway or the alternative complement pathway. In some cases, the target molecule is a growth factor or cytokine involved in the formation, growth and/or stabilization of new or existing blood vessels. The target molecule is not limited to these examples, and essentially any molecule can be a target molecule.

The term “competitor” or “competitor molecule” as used herein refers to any molecule that has a known functional or binding activity on a target molecule and that can be utilized to competitively displace an aptamer or other molecule from its binding site on the target molecule. The competitor molecule may generally bind to the target molecule at a site in which an aptamer is being generated and selected for according to the methods provided herein. The competitor molecule can be any substance including a protein, an antibody or antibody fragment, a peptide, a small molecule, a lipid, and the like. In some cases, the competitor molecule is a known therapeutic agent such as an agent currently used to treat a disease or disorder or an agent currently in development for the treatment of a disease or a disorder (e.g., a clinically-relevant drug). Generally, the competitor binds to a desired epitope of a target molecule, for example, an epitope that is therapeutically relevant (e.g., a bioactive epitope).

The term “epitope” as used herein refers to the part of an antigen (e.g., a substance that stimulates an immune system to generate an antibody against) that is specifically recognized by the antibody. In some cases, the antigen is a protein or peptide and the epitope is a specific region of the protein or peptide that is recognized and bound by an antibody. Often, the therapeutic aptamers generated herein specifically bind to therapeutically relevant or bioactive epitopes (e.g., epitopes that have a known function in the pathology of a disease or disorder).

The term “exosite” as used herein may refer to a domain or region of enzyme that is capable of binding to another protein. The exosite may also be referred to herein as a “secondary binding site”, for example, a binding site that is remote from or separate from a primary binding site (e.g., an active site). In some cases, the primary and secondary binding sites may overlap. Binding of a molecule to an exosite may cause a physical change in the enzyme (e.g., a conformational change). In some cases, the activity of an enzyme may be dependent on occupation of the exosite. In some examples, the exosite may be distinct from an allosteric site.

The term “catalytic cleft” or “active site” as used herein refers to a domain of an enzyme in which a substrate molecule binds to and undergoes a chemical reaction. The active site may include amino acid residues that form temporary bonds with the substrate (e.g., a binding site) and amino acid residues that catalyze a reaction of that substrate (e.g., catalytic site). The active site may be a groove or pocket (e.g., a cleft) of the enzyme which can be located in a deep tunnel within the enzyme or between the interfaces of multimeric enzymes.

With respect to the described methods, a competitor may be capable of binding to a target molecule at a “target epitope”. For example, the target epitope may be an exosite or a catalytic cleft of an enzyme. In another example, the target epitope may be the portion of a cytokine or growth factor that binds to the cognate receptor(s) of said cytokine or growth factor. Alternatively, the target epitope may be the portion of a receptor that binds to the cognate cytokine or growth factor which signals via the receptor. The target epitope may be a portion of a cytokine, a growth factor or a receptor for a cytokine or growth factor that, when the cytokine or growth factor is in complex with its cognate receptor, is responsible for binding to an accessory protein necessary for the cytokine or growth factor-receptor complex to generate a complex capable of transducing a signal. Generally, the target epitope is an epitope having therapeutic relevance, for example, a bioactive epitope.

The terms “subject” and “patient”, to the extent used herein, are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

Described herein are methods for generating and selecting aptamers. In some cases, the aptamers can be used as therapeutic agents to treat, e.g., a disease or a disorder. In some cases, the methods described may be more effective or more efficient at selecting a therapeutic aptamer than traditional aptamer selection methods. The methods may be used to select for aptamers with a specific binding characteristic or a specific function. For example, in some cases it may be desirable to generate an aptamer that inhibits the function of a target protein. The methods can be utilized to specifically select for aptamers that inhibit the function of the target protein. The methods provided herein generally include a step of competitive displacement to specifically select for those aptamers with desired binding capabilities. The competitive displacement steps may involve the use of a competitor molecule to competitively displace an aptamer from a binding site of the target molecule. By using iterative rounds of competitive selection, aptamers with desired functions or binding characteristics can be selected. Alternatively, desired aptamers can be generated by using the competitor molecule to deplete aptamers with the desired function by pre-binding the competitor to the target to mask the desired binding site. Iterating rounds of selection with an aptamer library already enriched for aptamers to the target using the competitor-target complex may reduce the frequency of, or de-enrich, for aptamers that bind the desired epitope on the target. Following this selection procedure, such aptamers can be identified by comparing sequences present in aptamer libraries subjected to cycles of de-enrichment as compared to the aptamer library enriched to the target prior to the de-enrichment selection process. Alternatively, desired aptamers can be generated without the need for iterative amplification and enhancement, e.g., by performing different, parallel screens of the initial aptamer library against competitive agent(s) and other reference conditions and using next-generation sequencing to identify those aptamers that optimally and uniquely bind to and compete for a specific target epitope. Often, the methods involve bioinformatics approaches to identify aptamers that are enriched (or depleted in methods involving de-enrichment) in response to selective pressure with a clinically-relevant drug. Aptamer sequences present in a final aptamer pool (e.g., after performing one or more rounds of selection as described herein) may be compared to a starting aptamer pool (e.g., prior to performing one or more rounds of selection as described herein) and those aptamers that are enriched in the final pool relative to the starting pool may be identified. Further, the relative rate of enrichment of these aptamer sequences can be assessed by comparing the enrichment (or depletion if using de-enrichment methods) of these aptamer sequences in pools that are not subjected to selective pressure versus those pools that are. Those aptamers that are enriched in the final pool and demonstrate a high rate of enrichment under selective pressure may be identified as candidate therapeutic aptamers. After candidate therapeutic aptamers are identified, these aptamers may be tested in functional assays that assess the bioactivity and the clinical relevance of these aptamers.

The following description provides various embodiments of the methods. It should be understood that any combination of the steps described herein may be performed and the ordering of the steps performed may vary slightly between methods. In some cases, not all steps need be performed to practice the invention. Specific examples have been provided.

### Aptamer Libraries

The methods herein provide for generating an aptamer library. The aptamer library may be screened for the selection of one or more therapeutic aptamers using the methods as detailed herein. The aptamer library may include DNA aptamers, RNA aptamers or a combination thereof. In some cases, the DNA aptamers are modified DNA aptamers. In some cases, the RNA aptamers are modified RNA aptamers. RNA or DNA aptamers can include any number of modifications that may protect the aptamer from nuclease degradation or enhance the stability of the aptamer under physiological conditions. For example, the RNA aptamers may include one or more 2′ 0-Methyl modifications (2′OMe) or 2′ fluoro modifications (2′F). The library aptamers may include any modifications as described herein.

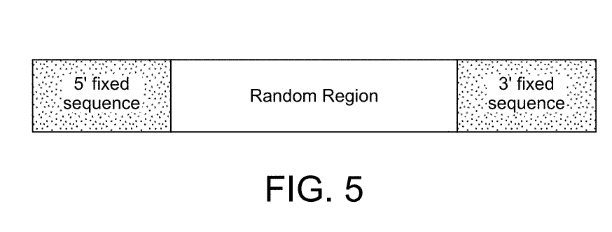

The library may contain at least about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸, about 10¹⁹, about 10²⁰ or greater than about 10²⁰ different aptamers. An example of a modified library aptamer that is suitable for performing the methods described herein is depicted in FIG. 5.

The aptamers in the aptamer library may include a random sequence of nucleotides. The random sequence of nucleotides may be of any length. In some cases, the random sequence of nucleotides may be about 20 to about 80 nucleotides in length. For example, the random sequence can be about 20, about 25, about 30, about 35, about 40, about 45, about 50, about 55, about 60, about 65, about 70, about 75, about 80, or greater than 80 nucleotides in length. The random sequence may include the portion of the aptamer that binds to the target molecule. The random sequence may include any combination of the standard nucleobases adenine (A), cytosine (C), guanine (G), thymine (T), or uracil (U). The random sequence may also include any number and combination of non-standard nucleobases or nucleic acid analogues, non-limiting examples including those produced by Tagcyx Biotechnologies (Japan) those described in U.S. Pat. No. 7,179,894 to Gorenstein et al., or those described in Georgiadis et al., 2015, J. Am. Chem. Soc. The random sequence may further comprise any number of aptamer modifications, as described herein.

The library aptamers may further include one or more conserved nucleotide regions. The one or more conserved nucleotide regions may be identical or substantially identical for each aptamer of the library. The one or more conserved nucleotide regions may be two conserved nucleotide regions that flank the aptamer. The one or more conserved nucleotide regions may include a primer sequence that can be used, for example, to prime an amplification reaction (e.g., polymerase chain reaction) or a sequencing reaction (e.g., sequencing-by-synthesis (SBS) reaction) or for reverse transcription of RNA-based libraries prior to an amplification reaction. In some cases, the one or more conserved nucleotide regions includes two nucleotide regions that function as a forward primer and a reverse primer. The one or more conserved nucleotide regions may be of any length, for example, about 5, about 6, about 7, about 8, about 9, about 10, about 11, about 12, about 13, about 14, about 15, about 16, about 17, about 18, about 19, about 20, about 21, about 22, about 23, about, 24, about 25, about 26, about 27, about 28, about 29, about 30, about 31, about 32, about 33, about 34, about 35, about 36, about 37, about 38, about 39, about 40, about 45, about 50 or greater than about 50 nucleotides in length.

The overall length of the library aptamer may be about 50 to about 800 nucleotides. For example, the overall length of the library aptamer may be about 50, about 100, about 150, about 200, about 250, about 300, about 350, about 400, about 450, about 500, about 550, about 600, about 650, about 700, about 750, about 800 or greater than about 800 nucleotides.

In some examples, the aptamer library is provided or generated in a solution. In alternative examples, the aptamer library may be immobilized to a solid support. Methods of immobilizing nucleic acids to solid supports are known in the art. Any number of chemistries or linking groups may be used to covalently attach nucleic acids to a solid support. In some cases, the library aptamers are attached to beads. Non-limiting examples of beads include polystyrene, magnetic, silica, or any combination thereof. The beads may have a functionalized surface (e.g., carboxylic acid, amine, or thiol surface chemistries) that may aid in the covalent bonding of nucleic acids. Each aptamer of the aptamer library may include one or more cleavable chemical bonds that may be chemically cleaved to release the aptamers into the solution phase to e.g., aid in the recovery of the desired aptamers.

### Target Molecules

The methods described herein provide for one or more selection methods. The selection methods generally involve the binding of an aptamer library to a target molecule to identify aptamers that have the ability to bind to the target. Target molecules can be, but are not limited to, proteins, peptides, nucleic acids (e.g., DNA or RNA), lipids, or even biological cells (e.g., bacterial cells). Often, the target molecule is known or suspected of playing a biological function in the pathology of a disease or disorder such that modulating the biological activity of the target molecule may alleviate, treat, or cure the disease or disorder. In some embodiments, the target molecules are immobilized to a solid support. In some cases, the target molecule is isolated (e.g., separated from its natural environment). In a non-limiting example, the target molecule is isolated or extracted from a biological cell, a tissue, a bodily fluid, or a biological matrix. In another non-limiting example, the target molecule is a recombinant protein (e.g., one that is produced using recombinant DNA techniques). Essentially any solid support may be used to immobilize a target molecule. The solid support may be selected based on the specific binding chemistry of the target molecule and the support. Solid supports may have functionalized surfaces to aid in the binding of a target molecule. Examples of solid supports can include, without limitation, beads as described above (e.g., polystyrene, silica, magnetic), flow columns, filters, and the like. Any known method of immobilizing a molecule to a solid support may be used. Binding of the molecule to the support can be covalent binding or non-covalent binding.

In some cases, the target molecule is a protein. The protein can be immobilized to a solid support by attaching one or more binding molecules to the protein. For example, a protein can include biotin which allows the protein to bind to streptavidin immobilized on a solid support. In some cases, the protein includes a 6× His-Tag (SEQ ID NO: 5) which can bind to an anti-His-Tag antibody immobilized on a solid support or a Ni-NTA resin. Other non-limiting examples of affinity tags that may be used include glutathione-S-transferase (GST) and maltose binding protein (MBP). It should be understood that the binding chemistry utilized may be dependent at least on the identity of the target molecule (e.g., protein or nucleic acid) and the support to be used (e.g., bead or filter).

The methods provided herein may improve the generation and selection of aptamers that have a specific functional activity (e.g., inhibition of a protein by a specific mechanism of action such as blocking the active site cleft) and as such, the target molecule used in the selection of the aptamers should retain its function. Thus, any method of immobilizing a target molecule to a solid support should be compatible with and not interfere with the function of the molecule. For example, if the target molecule is a protease, the protease should retain its proteolytic function during the selection process. Therefore, the immobilization process should be compatible with and not interfere with the proteolytic activity of the enzyme. Furthermore, as will be demonstrated herein, the immobilized molecule should also retain the ability to be bound by a competitor molecule. For example, if the target molecule is a protease, the protease should retain the ability (after immobilization) to be bound by the competitor that will be used during the selection process. Numerous methods may be used to ascertain whether the immobilized molecule retains functional activity and the ability to bind a competitor. Taking the example of a protease again, a substrate for the protease may be flowed onto the solid support and the ability of the protease to cleave the substrate is determined. Similarly, to test for competitor binding, a known amount of a competitor molecule can be flowed onto the support (e.g., column) and the amount of bound competitor can be determined relative to the amount of a bound control molecule. It should be understood that the aptamer library may be immobilized to a solid substrate and the target molecule can be provided in solution phase. The proceeding steps can be performed with either scenario.

### Methods

In some cases, the selection process may involve a negative selection step. Negative selection may be useful to e.g., remove any aptamers that have affinity for the solid support or partitioning matrix (e.g., nitrocellulose) used in the selection process. Generally, a negative selection step may be used prior to initiating the selection process to reduce the number of aptamers that have affinity for and that bind to the solid support or partitioning matrix. A negative selection step may comprise incubating the aptamer library with the solid support or partitioning matrix to be used in the downstream selection process. The solid support or partitioning matrix does not have any target molecules bound, such that any aptamers that bind to the solid support are non-specific binders. The incubation step may be performed at low-stringency such that aptamers with low-affinity for the solid support or partitioning matrix may be removed. After a sufficient incubation period, the solid support or partitioning matrix can be captured (e.g., when magnetic beads are used, a magnet may be applied) and the supernatant containing unbound aptamers can be collected and subjected to downstream selection processes. The negative selection step may be performed prior to every round of selection, at every other round of selection, or as needed based upon the appearance of enrichment of the aptamer library to the solid support or partitioning matrix.

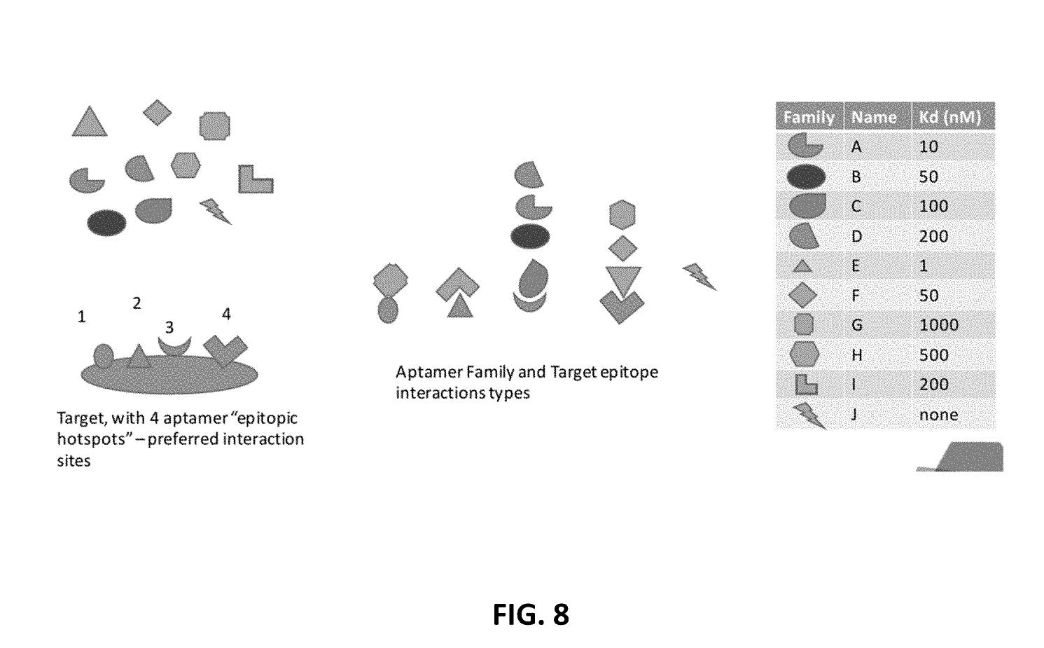

In some cases, the selection process involves one or more positive selection steps. The one or more positive selection steps may involve the exposure of the aptamer library to the immobilized target molecule (or an immobilized aptamer library can be exposed to a solution-phase target). Any aptamers that have the ability to bind to the target molecule, under the appropriate conditions, may bind to the target molecule. The aptamer library may be incubated with the target molecule for a sufficient time and under sufficient conditions for the aptamers to bind to the target molecule. At this step, any aptamers that bind to the target molecule can be selected for, and any aptamers that do not bind to the target molecule can be deselected for. It may be desirable to select for aptamers that specifically bind to the target molecule (e.g., that bind to a specific binding site on the target molecule). A target molecule may have any number of epitopes that can be recognized by an aptamer and during the positive selection step, any aptamers that bind to any epitopes found on the target molecule may be selected. At this stage then, aptamers may be selected that bind to desired epitopes as well as undesired epitopes. For example, FIG. 8 illustrates a target molecule with four different epitopes and different families of aptamers that can bind each epitope with varying affinities.

The aptamer library may be incubated with the target molecule under conditions suitable for binding of the aptamers to the target molecule. These conditions may vary and may be empirically determined. The conditions may be altered or adjusted as needed to increase or decrease the ability of the aptamers to bind to the target molecules. In some cases, the aptamers may be incubated with the target molecule under low stringency conditions (e.g., lower temperature, lower ionic strength, higher target concentration, etc.) to enable more aptamers to bind to the target molecule. In some cases, the aptamers may be incubated with the target molecules under high stringency conditions (e.g., higher temperature, higher ionic strength, lower target concentration, etc.) to enable only aptamers with high affinity for the target molecule to bind.

In some cases, the amount of aptamer library used is from about 10 pmole to about 10 nmoles. For example, the amount of aptamer library used is about 10 pmoles, about 25 pmoles, about 50 pmoles, about 75 pmoles, about 100 pmoles, about 200 pmoles, about 300 pmoles, about 400 pmoles, about 500 pmoles, about 600 pmoles, about 700 pmoles, about 800 pmoles, about 900 pmoles, about 1 nmole, about 2 nmoles, about 3 nmoles, about 4 nmoles, about 5 nmoles, about 6 nmoles, about 7 nmoles, about 8 nmoles, about 9 nmoles or about 10 nmoles. In some cases, the amount of target molecule used is from about 1 pM to about 20 μM. For example, the amount of target molecule may be about 1 pM, about 5 pM, about 10 pM, about 25 pM, about 50 pM, about 75 pM, about 100 pM, about 200 pM, about 300 pM, about 400 pM, about 500 pM, about 600 pM, about 700 pM, about 800 pM, about 900 pM, about 1 nM, about 10 nM, about 25 nM, about 50 nM, about 75 nM, about 100 nM, about 200 nM, about 300 nM, about 400 nM, about 500 nM, about 600 nM, about 700 nM, about 800 nM, about 900 nM, about 1 μM, about 5 μM, about 10 μM, about 15 μM, or about 20 μM.

In some cases, the aptamer library is incubated with the target molecule for a period of time sufficient to allow binding of the aptamers to the target molecule. The aptamer library may be incubated with the target molecule from about 5 minutes to about 120 minutes. For example, the aptamer library may be incubated with the target molecule for a period of 5, 10, 15, 20, 25, 30, 35, 40, 45, 50, 55, 60, 65, 70, 75, 80, 85, 90, 95, 100, 105, 110, 115, or 120 minutes.

In some cases, the aptamer library is incubated with the target molecule at a pH of 5.0-9.0. For example, the aptamer library may be incubated with the target molecule at a pH of 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

In some cases, the aptamer library is incubated with the target molecule at a temperature from about 20° C. to about 60° C., from about 30° C. to about 50° C., or from about 35° C. to about 42° C. For example, the aptamer library may be incubated with the target molecule at a temperature of about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C.

In some cases, the aptamer library is incubated with the target molecule in a buffered solution. Any buffer may be suitable for positive selection including, but not limited to, phosphate-buffered saline (PBS), Tris-buffered saline (TBS), Borate-buffered saline, buffers containing Tween-20, buffers containing EDTA, MES, BIS-TRIS, ADA, ACES, BIS-TRIS PROPANE, PIPES, ACES, MOPSO, Cholamine chloride, MOPS, BES, TES, HEPES, DIPSO, MOBS, TAPSO, Acetamidoglycine, TAPSO, TEA, POPSO, HEPPSO, EPS, HEPPS, Tricine, TRIZMA, Glycinamide, Glycyl-glycine, HEPBS, Bicine, TAPS, AMPB, CHES, AMP, AMPSO, CAPSO, CAPS and CABS.

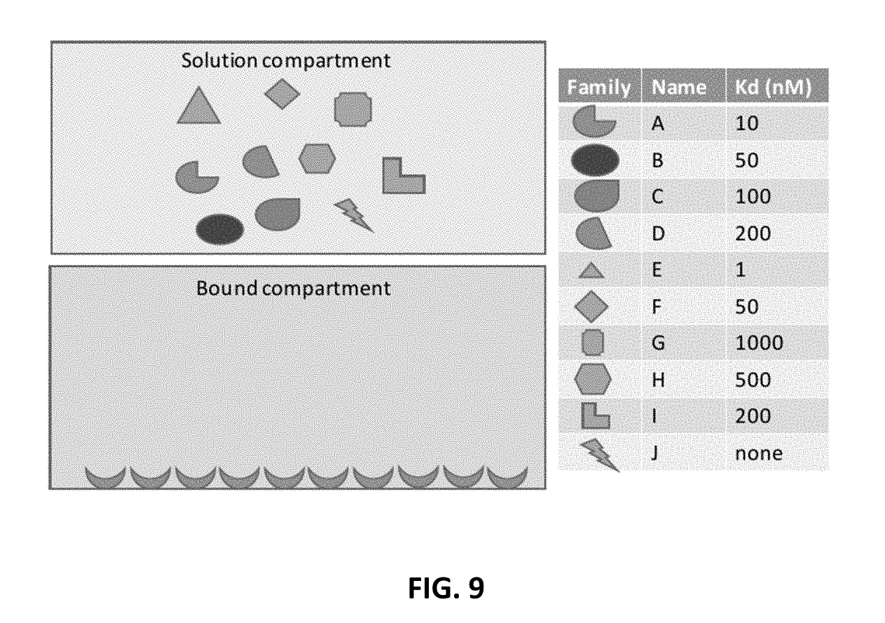

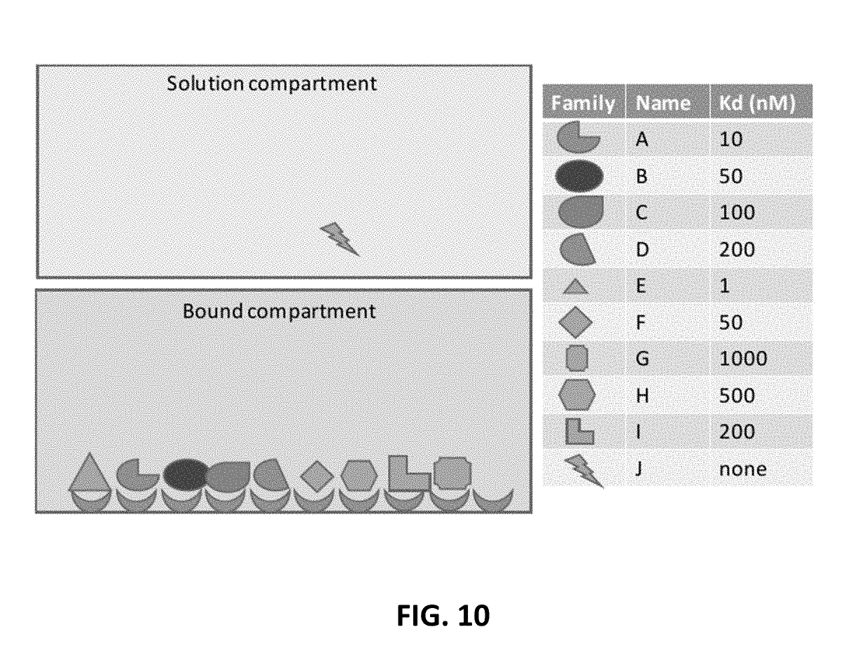

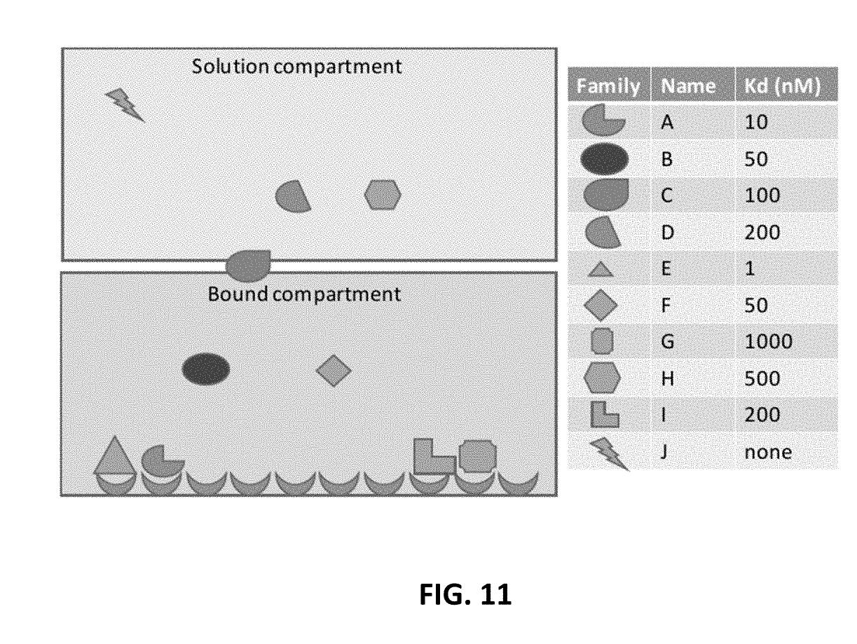

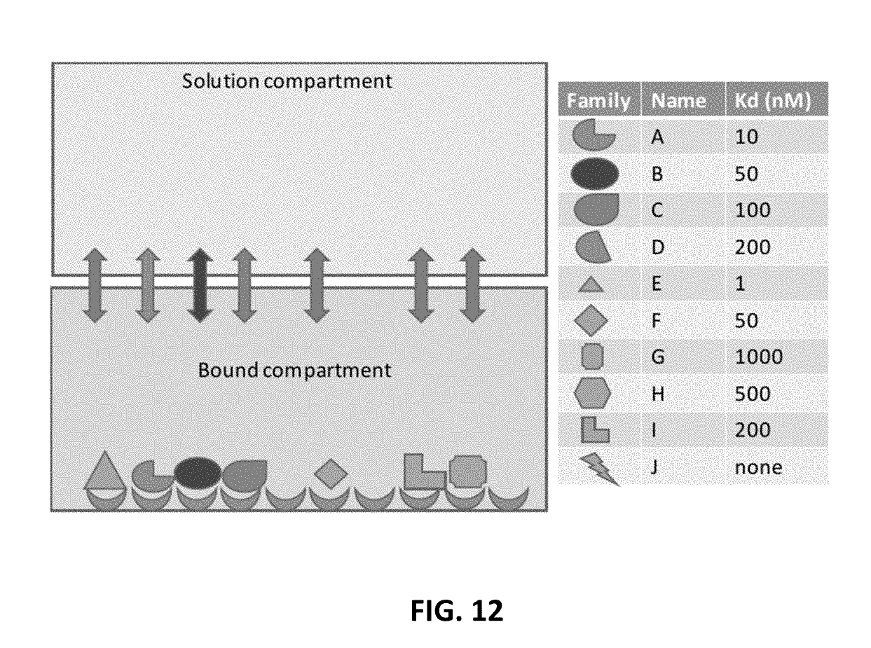

In initial rounds of selection it may be important to ensure that rare epitope-binding molecules are recovered. Often an aptamer library may have a sequence diversity on the order of about 10¹⁰, 10¹¹, 10¹², 10¹³, 10¹⁴, 10¹⁵, 10¹⁶, 10¹⁷, 10¹⁸, 10¹⁹, 10²⁰ or greater. In such aptamer libraries, sequence copy number may be low, for example, on the order of 1, 2, 3, 4, 5, 6, 7, 8, 9, 10, 11, 12, 13, 14, 15, 16, 17, 18, 19, or 20 sequence copy numbers. In such cases, positive selection may involve contacting an aptamer library with a high concentration of target molecule such that the number of available epitopes on the target molecule is not limiting. FIGS. 9-12 illustrate this concept. In this non-limiting example, an aptamer library of high diversity and low sequence copy number is provided to a target molecule containing four epitopes per target molecule. In this example, because the concentration of target molecule is high, there may be enough epitopes available on the target molecules for every aptamer that is capable of binding to one of the four epitopes on the target molecule to bind regardless of the binding affinity. Immediately after the aptamer library is added to the target molecule, as depicted in FIG. 9, none of the aptamers are yet bound to the target molecules. After a suitable incubation period, as depicted in FIG. 10, all of the aptamers with binding affinity for one of the four epitopes are bound to the target molecule, whereas those aptamers that do not have binding affinity for any of the four epitopes may remain in the solution. At equilibrium, however, there may be a distribution of aptamers found in the solution compartment and the bound compartment (FIG. 11). Washing away the solution at this stage may leave behind those aptamers that have a higher affinity for one of the four epitopes on the target molecules, however, aptamers with lower binding affinity may also be selected for (FIG. 12).

In some cases, a wash step may be performed after each positive selection step. The wash step may generally leave the higher affinity aptamers bound to the target molecule and remove any unbound aptamers and/or lower affinity aptamers. Wash steps may utilize a variety of volumes or durations to ensure sufficient stringency. Wash steps may utilize a variety of wash buffers of different ionic strengths, temperatures and pHs to ensure sufficient stringency. Wash steps may utilize different wash buffers depending on the stage in which they are performed in the method. For example, early wash steps may be performed with wash buffers of low ionic strength to remove only unbound aptamers while later steps may use wash buffers with high ionic strength to remove weakly bound aptamers. The choice of wash buffer can determine the stringency of the wash step and can be used to select for or against different aptamers. Non-limiting examples of wash buffers include Phosphate-buffered saline (PBS), Tris-buffered saline (TBS), Borate-buffered saline, buffers containing Tween-20, buffers containing EDTA, MES, BIS-TRIS, ADA, ACES, BIS-TRIS PROPANE, PIPES, ACES, MOPSO, Cholamine chloride, MOPS, BES, TES, HEPES, DIPSO, MOBS, TAPSO, Acetamidoglycine, TAPSO, TEA, POPSO, HEPPSO, EPS, HEPPS, Tricine, TRIZMA, Glycinamide, Glycyl-glycine, HEPBS, Bicine, TAPS, AMPB, CHES, AMP, AMPSO, CAPSO, CAPS and CABS.

In some cases, it may be desirable to collect the aptamers bound to the target molecule for further downstream processing or analysis. In one non-limiting example, an aptamer library is provided wherein each aptamer is immobilized on a solid support such as a bead. As previously described, each immobilized aptamer may include a cleavable chemical bond. Upon addition of an appropriate stimulus, the chemical bond can be cleaved and the aptamer released from the solid support.

After positive selection, the target molecule complexes may be washed as above to remove any unbound aptamers, the bound aptamers may be recovered from the target molecule as above to generate an enriched pool of aptamers, and the enriched pool of aptamers may be amplified. After performing several rounds of positive selection followed by recovery and amplification, the frequency of low copy number binders may be increased.

One or more rounds of positive selection may be performed. In some cases, more than one positive selection steps may be performed, for example, in some cases, 2, 3, 4, 5, 6, 7, 8, 9, 10 or more than 10 positive selection steps may be performed. The positive selection steps may comprise similar reaction conditions or they may differ substantially. In one non-limiting example, the stringency of the reaction conditions may be increased with each successive round of positive selection to further select for high affinity aptamers. Any combination of reaction conditions may be utilized to obtain the desired aptamers and may be empirically determined.

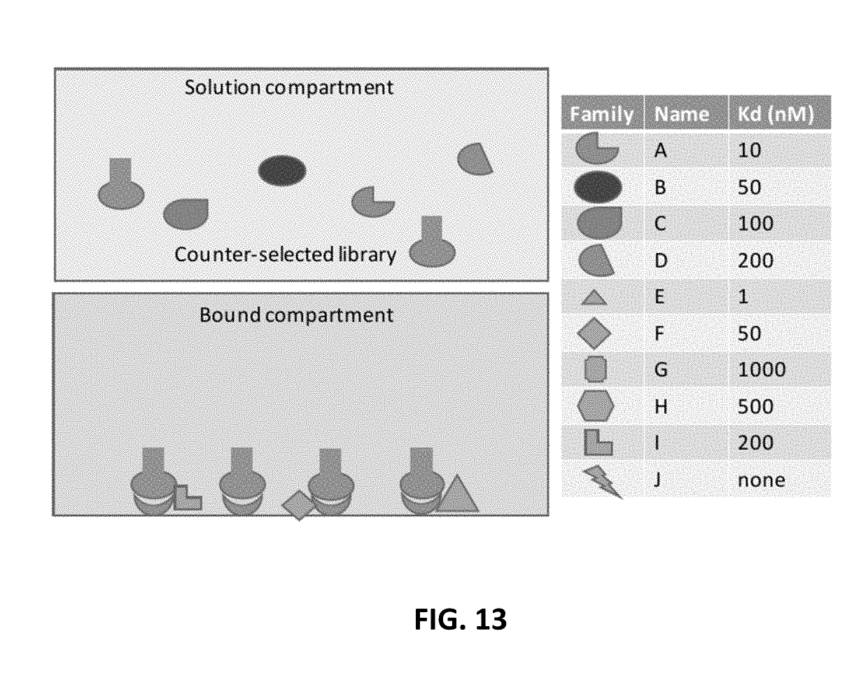

Often, it may be desirable to enrich the aptamer library or pool for target epitope-binding aptamers. For example, it may be desirable to remove any aptamers that bind to the target molecule at non-target epitopes. In some cases, one or more counter-selection methods may be performed. In some cases, counter-selection may comprise adding a competitor that is capable of specifically binding to the target epitope of the target molecule prior to the addition of the aptamer library or pool. Often, the competitive binder is any molecule that can specifically bind to the target molecule at a desired epitope. For example, in some cases, the competitive binder may be an antibody or antibody fragment, small molecule, or peptide that has high binding affinity for a desired epitope on the target molecule. Without wishing to be bound by theory, the competitive binder may mask the desired epitopes on the target molecule such that the aptamers that bind to the desired epitopes cannot bind, whereas those aptamers that bind to other regions of the target molecule can bind. (See FIG. 13) The competitive binder may be incubated with the target molecule for a period of time prior to the addition of the aptamers. The reaction conditions are generally sufficient for the competitive binder to bind the target molecule.

In some cases, the competitive binder may be added to the target molecule at a molar excess to ensure that the majority of the epitopes on the target molecule are bound by competitive binder. For example, the competitive binder may be added to the target molecule at a concentration that is at least 1.1×, 1.2×, 1.3×, 1.4×, 1.5×, 1.6×, 1.7×, 1.8×, 1.9×, 2.0×, 2.5×, 3.0×, 3.5×, 4.0×, 4.5×, 5.0×, 5.5×, 6.0×, 6.5×, 7.0×, 7.5×, 8.0×, 8.5×, 9.0×, 9.5×, 10.0×, 11.0×, 12.0×, 13.0×, 14.0×, 15.0×, 16.0×, 17.0×, 18.0×, 19.0×, 20.0×, 21.0×, 22.0×, 23.0×, 24.0×, 25.0×, 26.0×, 27.0×, 28.0×, 29.0×, 30.0×, 31.0×, 32.0×, 33.0×, 34.0×, 35.0×, 36.0×, 37.0×, 38.0×, 39.0×, 40.0×, 41.0×, 42.0×, 43.0×, 44.0×, 45.0×, 46.0×, 47.0×, 48.0×, 49.0×, 50.0×, greater than the concentration of the target molecule.

After the desired epitopes are blocked by the competitive binder, the aptamers or aptamer library may be applied to the sample. Any aptamers that can bind to regions of the target molecule that are not blocked by the competitive binder may bind to the target molecule, where as any aptamers that bind to the epitope that is blocked by the competitive binder may remain in the solution compartment. If the target molecule is immobilized to a solid support, the solid support may be partitioned (e.g., by magnet for magnetic beads) and the solution may be collected (e.g., by aspirating or decanting) to a clean tube. In some cases, the target molecule/competitive binder/aptamer complexes may be collected for further downstream processing. In some cases, one or more wash steps may be performed as previously described to remove any unbound aptamers. In some cases, the wash steps are performed with low stringency to ensure that any aptamers that are bound to the target molecule remain bound. In some instances, after the unbound aptamers have been collected, any remaining competitive binder may be removed from the solution compartment (i.e., any competitive binder that did not bind the target molecule). Any method of removing competitive binders known in the art may be used and the method chosen may depend on the identity of the competitive binder used. In a non-limiting example, when the competitive binder is any antibody, bead-immobilized Protein A may be used to sequester and remove the antibody from the solution compartment.

In one instance, it may be desirable to further enrich an aptamer library or pool for those aptamers that have high affinity for the target epitope. In some cases, competitive elution may be performed to enrich for those aptamers that have high affinity for the desired epitope. In some cases, competitive elution involves the addition of a competitive binder to the target molecule after the aptamers have been bound to the target molecule. In some cases, competitive elution may be performed after one or more rounds of positive selection. In some cases, competitive elution may be performed after one or more rounds of counter-selection, followed by one or more rounds of positive selection. These steps may generally be performed in any order and any combination.

In some cases, competitive elution involves the incubation of aptamers with the target molecule under conditions that allow the aptamers to bind to the target molecule. In general, the competitor molecule may be incubated with the bound aptamer-target molecule at a high molar excess of competitor molecule to allow the competitor molecule to compete off and displace any aptamer that shares a binding site with the competitor. The competitor molecule that may be selected is based on possessing an analogous function to that which the aptamer library is being screened for. For example, if an aptamer that inhibits an enzyme is desired, a competitor molecule that inhibits that same enzyme may be used. Similarly, if an aptamer that binds an enzyme at a specific binding site is desired, a competitor molecule that binds to that same enzyme at that specific binding site may be used. In one specific but non-limiting example, if an aptamer that inhibits the function of the alternative complement pathway enzyme Factor D is desired, an anti-Factor D antibody or Fab with inhibitory activity against Factor D could be used as a competitor molecule, such as an anti-Factor D Fab having an amino acid sequence of heavy chain variable region of: EVQLVQSGPELKKPGASVKVSCKASGYTFTNYGMNWVRQAPGQGLEWMGWINTY TGETTYADDFKGRFVFSLDTSVSTAYLQISSLKAEDTAVYYCERGGVNNWGQGTLV TVSSASTKGPSVFPLAPSSKSTSGGTAALGCLVKDYFPEPVTVSWNSGALTSGVHTFP AVLQSSGLYSLSSVVTVPSSSLGTQTYICNVNHKPSNTKVDKKVEPKSCDKTHT (SEQ ID NO:1) and an amino acid sequence of light chain variable region of:

(SEQ ID NO: 2)
DIQVTQSPSSLSASVGDRVTITCITSTDIDDDMNWYQQKPGKVPKLLISG
GNTLRPGVPSRFSGSGSGTDFTLTISSLQPEDVATYYCLQSDSLPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

The examples of target and competitor molecules highlighted throughout this disclosure are not to be considered preferred or exhaustive; any known molecule with a desired functional or binding characteristic could be used as a competitor molecule. Non-limiting examples of competitor molecules may include proteins, peptides, antibodies, nucleic acids, other aptamers, small molecules, lipids, and the like. In some cases, the competitor molecule is a known therapeutic agent either currently used for the treatment of a disease or disorder or in clinical development for the treatment of a disease or disorder. Generally, the competitor binds to an epitope of a target molecule with known therapeutic relevance, such as a bioactive epitope.

Essentially any target and competitor pair can be utilized in this process. Non-limiting examples of target molecule and competitor [in brackets] pairs include Factor D [a therapeutically-relevant Fab to Factor D with an amino acid sequence of heavy chain variable region according to SEQ ID NO:1 and of light chain variable region according to SEQ ID NO:2; MAb 166-32; LS-C1357351; Factor P [anti-fP antibodies such as those produced by Novelmed or Novartis]; VEGF [Ranibizumab; or a therapeutically-relevant MAb to the Receptor-binding domain (RBD) of VEGF with an amino acid sequence of heavy chain variable region according to SEQ ID NO:3 and of light chain variable region according to SEQ ID NO:4; Aflibercept; abcipar pegol]; PDGF [Fovista; anti-PDGF antibodies such as those produced by Regeneron or Novartis]; Angiopoeitin 2 [anti-Ang2 antibodies or Fabs such as those produced by Genentech or Regeneron]; CTLA-4 receptor [Yervoy]; PD-1 and PDL-1 [Keytruda; PD Opdivo; atezolizumab; durvalumab]; amyloid-β [solanezumab; aducanumab; crenezumab]; interleukin 4 and interleukin 13 [Dupilumab]; LINGO-1 [Anti-LINGO-1; Biogen Idec]; PSK9 [antibodies such as those produced by Regeneron; evolocumab; alirocumab]; C5 [eculizumabl; anti-C5 antibodies such as those produced by Novartis]; and Interleukin-6 [Siltuximab; sirukumab; olokizumab].

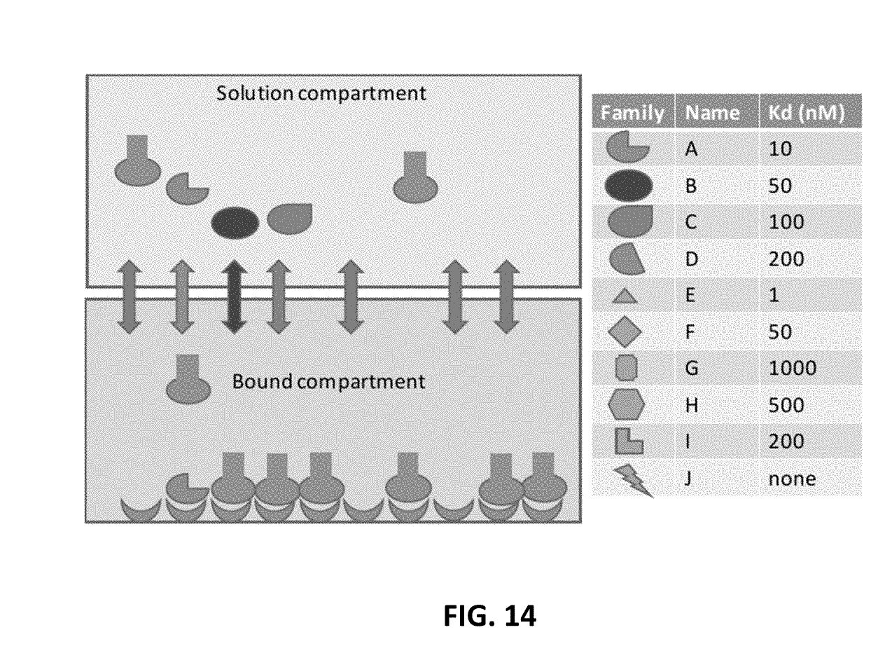

The competitor may have any affinity for the target molecule. In some cases, the competitor may have a high (pM to single-digit nM), a low (μM and greater) or a moderate (10s to 100s of nM) affinity for the target molecule. In some cases, the competitor may have higher affinity for the target molecule than the average binding affinity of the aptamers for the target molecule. After addition of the competitor, aptamers that share the same or similar epitope on the target molecule but with lower affinity for the epitope than the competitor may be displaced and be predominantly in the solution compartment. After addition of the competitor, aptamers that share the same or similar epitope on the target molecule but that have equal or greater affinity for the epitope than the competitor may be predominantly in the target-bound compartment. FIG. 14 illustrates this concept. As depicted, after addition of the competitor, lower affinity aptamers are competitively displaced from the epitope, however, those aptamers that have an equal or higher affinity for the epitope than the competitor (e.g., K_{d}=10 nM), may remain bound to the target molecule.

In some cases, the amount of competitor to be added can be determined based on the affinity of the competitor for the epitope. For example, if the competitor has low affinity for the epitope, the amount of the competitor added may be higher than for a competitor that has high affinity for the epitope. Generally, the amount of competitor added should be weighed relative to its binding affinity for the epitope such that the competitor competes with the bound aptamers.

In some cases, the competitor is added at a high molar excess. For example, the competitor may be added to the target molecule at a concentration that is at least 1.1×, 1.2×, 1.3×, 1.4×, 1.5×, 1.6×, 1.7×, 1.8×, 1.9×, 2.0×, 2.5×, 3.0×, 3.5×, 4.0×, 4.5×, 5.0×, 5.5×, 6.0×, 6.5×, 7.0×, 7.5×, 8.0×, 8.5×, 9.0×, 9.5×, 10.0×, 11.0×, 12.0×, 13.0×, 14.0×, 15.0×, 16.0×, 17.0×, 18.0×, 19.0×, 20.0×, 21.0×, 22.0×, 23.0×, 24.0×, 25.0×, 26.0×, 27.0×, 28.0×, 29.0×, 30.0×, 31.0×, 32.0×, 33.0×, 34.0×, 35.0×, 36.0×, 37.0×, 38.0×, 39.0×, 40.0×, 41.0×, 42.0×, 43.0×, 44.0×, 45.0×, 46.0×, 47.0×, 48.0×, 49.0×, 50.0×, 55.0×, 60.0×, 65.0×, 70.0×, 75.0×, 80.0×, 85.0×, 90.0×, 95.0×, or 100.0× greater than the concentration of the target molecule.

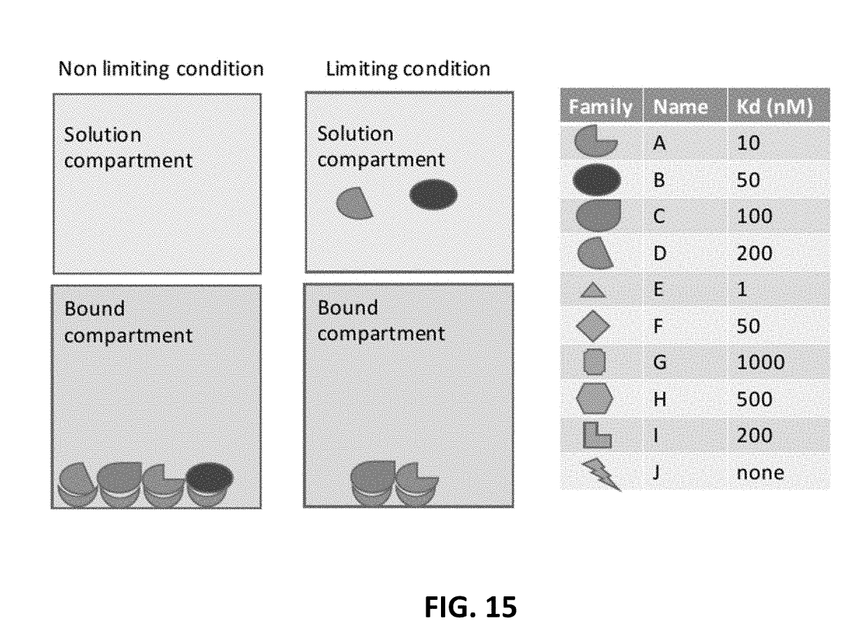

Additionally or alternatively, high affinity binders may be selected by limiting the amount of target molecules present in a positive selection step to eliminate lower affinity aptamers and enrich for higher affinity binders. The concentration of target molecules relative to the concentration of aptamers in the positive selection step may be at a ratio of about 5:1 to about 1:1,000. For example, FIG. 15 demonstrates that higher affinity binders may be selected for by limiting the concentration of target molecule present during the positive selection step. As depicted, under non-limiting conditions, high and low affinity binders may bind to the target molecules. However, when the target molecules are limited, the higher affinity binders may act as competitors and preferentially bind to the target molecules, thereby preventing the lower affinity binders from binding. Those aptamers that bind to the target molecule under the situations may be recovered, thus enriching for higher affinity binders.

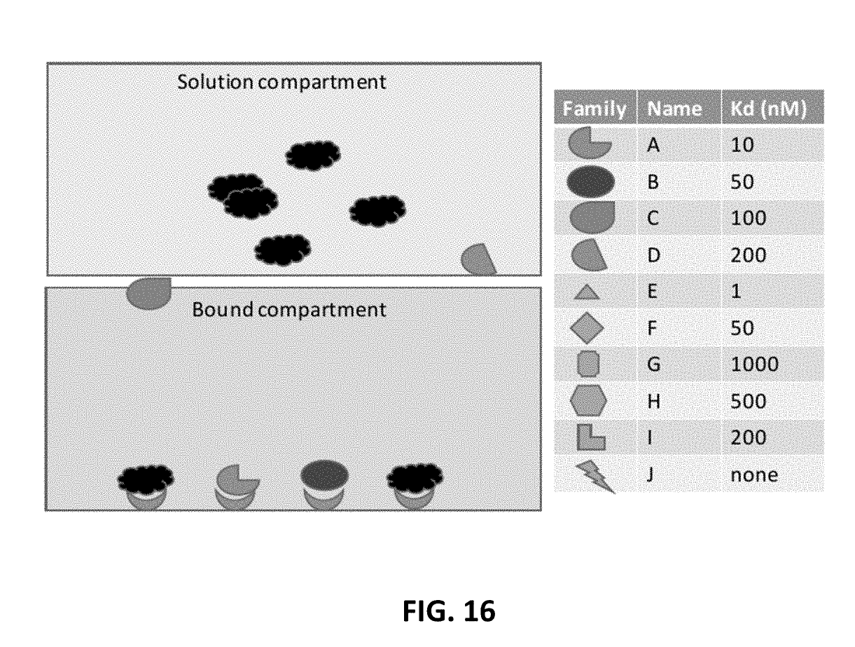

Alternatively or additionally, higher affinity aptamers may be selected for by the addition of a non-specific binder. The non-specific binder may be any molecule that non-specifically binds to the target molecule. Non-limiting examples of non-specific binders that may be utilized include: anionic polymers such as tRNA, dextran sulfate, heparin sulfate, hyaluronic acid, salmon sperm DNA, or other suitable polyanionic polymers. Without wishing to be bound by theory, the non-specific binder may non-specifically bind to the target molecule thus limiting the number of epitopes available on the target molecule. In such cases, those aptamers with high affinity may outcompete lower affinity aptamers for the target epitope, as illustrated in FIG. 16. In this example, only the high affinity aptamers may remain bound to the target molecules.

In some cases, the methods provided herein may select for aptamers that have high binding affinity for a target epitope present on the target molecule. The dissociation constant (K_{d}) can be used to describe the affinity of an aptamer for a target epitope (or to describe how tightly the aptamer binds). The dissociation constant may be defined as the molar concentration at which half of the binding sites of a target are occupied by the aptamer. Thus, the smaller the K_{d}, the tighter the binding of the aptamer to its target. The methods provided herein may preferentially select for aptamers that have a K_{d} of less than about 500 nM, 450 nM, 400 nM, 350 nM, 300 nM, 250 nM, 200 nM, 150 nM, 100 nM, 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 15 nM, 10 nM, 5 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 5 pM, or 1 pM. In some instances, the methods provide herein may preferentially select for aptamers that have a K_{d} of less than about 100 nM, 50 nM, 10 nM, or 5 nM.

Generally, the incubation conditions may be optimized to ensure maximal elution of the epitope-bound aptamers. In some situations, it may be more challenging to recover those aptamers with highest affinity for the target molecule or those aptamers with slow off-rates. In such cases, the kinetics and affinity of the aptamers for the target epitope may need to be considered to ensure maximal recovery of desired aptamers. For example, in competitive elution methods, in order for an aptamer to be outcompeted by the competitor for the target molecule, the aptamer must initially dissociate from the target epitope. For slow off-rate aptamers, the incubation period of competitor with aptamer-target molecule complex may need to be longer to ensure the bound aptamer is able to dissociate. In such cases where the incubation period is not sufficient, slow off-rate aptamers may ultimately be lost. For example, Example 8 depicts a method of antibody elution modeling to determine sufficient incubation times to maximize recovery of various compounds.

In some instances, aptamers that dissociate from a target molecule may be able to rebind to the target epitope, thereby resulting in lower chances of recovery. Thus, methods may be provided to prevent the aptamer from re-binding to the target epitope after dissociation. The methods herein provide sufficient incubation periods such that slow off-rate aptamers may be maximally recovered and to prevent aptamers from re-binding to the target epitope after dissociation.

In a non-limiting example, high concentrations of competitor may be used during competitive elution steps. In some cases, the ratio of competitor to aptamer may be at least 5:1, 10:1, 50:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10,000:1 or greater. In some instances, the ratio of competitor to aptamer is at least 1000:1. In some cases, incubation periods may be adjusted or altered to maximize the recovery of high affinity aptamers. For example, in some cases, the incubation period during a competitive elution step may be at least 30 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, or greater. In some instances, the incubation period during a competitive elution step is about 120 minutes or less.

In some examples, two or more iterative rounds of competitive elution may be used and the aptamers recovered in each round may be pooled. With each successive round of competitive elution, the same competitor and conditions may be used in each round, or in some cases, the concentration of competitor may be increased or a different competitor with a higher affinity for the target epitope may be used to increase the stringency of the elution process.

In some examples, the competitor molecule is provided at various concentrations or for various time periods. For example, the bound aptamer-target molecule can be aliquoted into a plurality of aliquots and each aliquot can be exposed to a different concentration of the competitor molecule. This method may be used to screen for aptamers that require a high concentration of competitor for displacement (i.e., have high affinity for the target). In another example, the aliquots can be exposed to the competitor molecule for various time periods. This method may be used to screen for aptamers that require a long incubation period with the competitor for displacement to occur.

In some examples, different competitor molecules each with a different affinity for the same or similar epitope may be used to competitively displace aptamers from the target molecule. For example, successive rounds of incubation may be performed with competitors each having a different binding affinity for the epitope. Lower binding-affinity competitors may be used first, and with each successive round of selection (i.e., binding to target molecule, washing, competitive elution), higher binding-affinity competitors may be used in subsequent selection rounds to increase the stringency of the selection process.

The methods further provide for one or more bioinformatics steps to identify aptamers with therapeutic relevance from a pool of aptamers. Generally, the bioinformatics steps involve comparing the aptamer sequences (identified using standard next-generation sequencing methods) present in a final aptamer pool (i.e., after performing one or more rounds of selective pressure as described herein) with a starting (parental) aptamer pool. For example, an aptamer library enriched by positive selection or prior to performing selective pressure methods may be the parental round against which subsequent selective pressure rounds are compared. In some instances, the enrichment of each aptamer sequence present in the final aptamer pool is calculated. Enrichment may be calculated as the fraction of an individual aptamer sequence present in the final aptamer pool divided by the fraction of the individual aptamer sequence present in the parental aptamer pool. Those aptamer sequences that demonstrate greater than 10-100× enrichment in the final aptamer pool may define a query set of sequences that are enriched in response to selective pressure with a bioactive epitope competitor. In situations in which a de-enrichment strategy is used, depletion in the final aptamer pool may be assessed. In some instances, the methods further involve comparing rates of enrichment between a final aptamer pool that has undergone selective pressure with control aptamer pools or aptamer pools that have not undergone selective pressure. In such cases, aptamers of interest may be selected by identifying those aptamers that have a higher rate of enrichment in aptamer pools subjected to selective pressure versus aptamer pools not subjected to selective pressure. These methods may be particularly suited to identify aptamers that are present in very low amounts throughout the selection process, whereas these aptamers may ordinarily be lost during traditional selection methods.

The methods provided herein further include testing a subset of candidate aptamers in functional assays. Functional assay may be performed in vitro such as in cell-based assay or cell-free assays. Generally, the functional assay is selected such that a biological activity of the candidate aptamers can be assessed. In a non-limiting example, if it is desired that a therapeutic aptamer inhibits the enzymatic activity of an enzyme by binding to the active site of the enzyme, the functional assay may preferentially test the ability of the candidate aptamer to bind to the active site and inhibit the enzymatic activity of the enzyme. Aptamers that exhibit desired bioactive properties in functional assays may be further screened with in vivo assays (e.g., animal models, disease models).

### Partitioning

Standard methods of partitioning (or separating) may be used after any step in the process, in any combination and as many times as deemed necessary. For example, partitioning methods may be used after negative selection steps, positive selection steps, counter-selection steps, wash steps, competitive elution steps, and any other steps disclosed herein. In some cases, partitioning methods may be used to separate one compartment from another (e.g., to separate the solid support from the solution). Examples of partitioning methods may depend on the solid support used. For example, magnetic beads can be separated by applying a magnetic source to the sample. In another example, a column can be washed and then the immobilized aptamer-target molecule can be eluted from the column. Other non-limiting examples of partitioning may include nitrocellulose filter binding, electrophoretic separation, microscopy (e.g., Atomic Force Microscopy), microfluidic-based partitioning, microarray-based partitioning or a well plate. The unbound aptamers can be discarded, may be reapplied to target molecules for further selection, or may be collected for downstream processing such as next-generation sequencing. The steps of binding and selection may be repeated as many times as is deemed necessary, in some cases, one, two, three, four, five or more than five times. In some cases, the aptamers and target molecules are incubated in solution phase. In this scenario, the bound aptamer-target molecules can be immobilized to a solid substrate after binding has taken place. After binding to the solid substrate, the immobilized aptamer-target molecules can be partitioned utilizing the techniques described above.

### Screening

Candidate therapeutic aptamers can be tested for functional or binding characteristics. Any method of testing an aptamer for functional or binding characteristics may be used. For example, surface plasmon resonance (SPR) may be used to measure binding affinity of the aptamer to a target molecule. SPR is a common technique that is well known in the art for measuring binding affinities of aptamers and antibodies to target molecules. In some cases, in vitro assays may be used to determine the activity of the aptamer. It will be understood that the appropriate in vitro assay to be used will depend at least on the specific target molecule, aptamer, and desired activity and one of skill in the art would be able to select the appropriate in vitro assay based on these criteria.

In another example, validation of binding to the specific/desired epitope may be performed by competitive SPR using the competitor agent, for example, by binding candidate aptamers to a plate and flowing the target over the candidate. The competitor can then be co-flowed in (i.e., target & competitor are flowed over the plate) at increasing concentrations to identify those sequences that compete best with the competitor. In other examples, functional assays can be performed to test the activity of the aptamer. In yet other examples, crystallographic techniques can be utilized.

Once representative structural motifs or sequences have been identified, they may be optimized through one or more optimization steps. The one or more optimization steps may increase the affinity or activity of the aptamer. The one or more optimization steps will generally be systematic and empirical. For example, a modification can be made to the motif or sequence and the resulting aptamer can be tested for affinity or activity. Any modification that reduces the affinity or activity of the aptamer may be discarded; any modification that improves the affinity or activity of the aptamer may be incorporated. In some cases, the minimal binding domains (the smallest number of nucleotides necessary for binding to the target molecule) can be determined using truncation assays, whereby the 5′ and 3′ ends of the aptamer are truncated one nucleotide at a time and an effect on affinity or activity is determined. In other examples, the one or more optimization steps may include linker scanning mutagenesis (e.g., replacing each nucleotide with a 3-carbon spacer and testing), secondary structure predicting algorithms including consideration of suboptimal folds, or doped selections (e.g., partially re-randomizing the sequence to build a library for use in further selection experiments). Any number of modifications can be made to the aptamers and are described below.

Aptamers as described herein may include any number of modifications than can affect the function or affinity of the aptamer. For example, aptamers may be unmodified or they may contain modified nucleotides to improve stability, nuclease resistance or delivery characteristics. In some cases, the aptamers described herein contain modified nucleotides to improve the affinity and specificity of the aptamers for a specific epitope, exosite or active site. Examples of modified nucleotides include those modified with guanidine, indole, amine, phenol, hydroxymethyl, or boronic acid. In other cases, nucleotide triphosphate analogs or CE-phosphoramidites may be modified at the 5′ position to generate, for example, 5-benzylaminocarbonyl-2′-deoxyuridine (BndU); 5-[N-(phenyl-3-propyl)carboxamide]-2′-deoxyuridine (PPdU); 5-(N-thiophenylmethylcarboxyamide)-2′-deoxyuridine (ThdU); 5-(N-4-fluorobenzylcarboxyamide)-2′-deoxyuridine (FBndU); 5-(N-(1-naphthylmethyl)carboxamide)-2′-deoxyuridine (NapdU); 5-(N-2-naphthylmethylcarboxyamide)-2′-deoxyuridine (2NapdU); 5-(N-1-naphthylethylcarboxyamide)-2′-deoxyuridine (NEdU); 5-(N-2-naphthylethylcarboxyamide)-2′-deoxyuridine (2NEdU); 5-(N-tryptaminocarboxyamide)-2′-deoxyuridine (TrpdU); 5-isobutylaminocarbonyl-2′-deoxyuridine (IbdU); 5-(N-tyrosylcarboxyamide)-2′-deoxyuridine (TyrdU); 5-(N-isobutylaminocarbonyl-2′-deoxyuridine (iBudU); 5-(N-benzylcarboxyamide)-2′-O-methyluridine, 5-(N-benzylcarboxyamide)-2′-fluorouridine, 5-(N-phenethylcarboxyamide)-2′-deoxyuridine (PEdU), 5-(N-3,4-methylenedioxybenzylcarboxyamide)-2′-deoxyuridine (MBndU), 5-(N-imidizolylethylcarboxyamide)-2′-deoxyuridine (ImdU), 5-(N-isobutylcarboxyamide)-2′-O-methyluridine, 5-(N-isobutylcarboxyamide)-2′-fluorouridine, 5-(N—R-threoninylcarboxyamide)-2′-deoxyuridine (ThrdU), 5-(N-tryptaminocarboxyamide)-2′-O-methyluridine, 5-(N-tryptaminocarboxyamide)-2′-fluorouridine, 5-(N-[1-(3-trimethylamonium)propyl]carboxyamide)-2′-deoxyuridine chloride, 5-(N-naphthylmethylcarboxyamide)-2′-O-methyluridine, 5-(N-naphthylmethylcarboxyamide)-2′-fluorouridine, 5-(N-[1-(2,3-dihydroxypropyO]carboxyamide)-2′-deoxyuridine), 5-(N-2-naphthylmethylcarboxyamide)-2′-O-methyluridine, 5-(N-2-naphthylmethylcarboxyamide)-2′-fluorouridine, 5-(N-1-naphthylethylcarboxyamide)-2′-O-methyluridine, 5-(N-1-naphthylethylcarboxyamide)-2′-fluorouridine, 5-(N-2-naphthylethylcarboxyamide)-2′-O-methyluridine, 5-(N-2-naphthylethylcarboxyamide)-2′-fluorouridine, 5-(N-3-benzofuranylethylcarboxyamide)-2′-deoxyuridine (BFdU), 5-(N-3-benzofuranylethylcarboxyamide)-2′-O-methyluridine, 5-(N-3-benzofuranylethylcarboxyamide)-2′-fluorouridine, 5-(N-3-benzothiophenylethylcarboxyamide)-2′-deoxyuridine (BTdU), 5-(N-3-benzothiophenylethylcarboxyamide)-2′-O-methyluridine, 5-(N-3-benzothiophenylethylcarboxyamide)-2′-fluorouridine; 5-[N-(1-morpholino-2-ethyl)carboxamide]-2′-deoxyuridine (MOEdu); R-tetrahydrofuranylmethyl-2′-deoxyuridine (RTMdU); 3-methoxybenzyl-2′-deoxyuridine (3MBndU); 4-methoxybenzyl-2′-deoxyuridine (4MBndU); 3,4-dimethoxybenzyl-2′-deoxyuridine (3,4DMBndU); S-tetrahydrofuranylmethyl-2′-deoxyuridine (STMdU); 3,4-methylenedioxyphenyl-2-ethyl-2′-deoxyuridine (MPEdU); 4-pyridinylmethyl-2′-deoxyuridine (PyrdU); or 1-benzimidazol-2-ethyl-2′-deoxyuridine (BidU); 5-(amino-1-propenyl)-2′-deoxyuridine; 5-(indole-3-acetamido-1-propenyl)-2′-deoxyuridine; or 5-(4-pivaloylbenzamido-1-propenyl)-2′-deoxyuridine. Aptamers as described herein may include any number of modifications that can affect the function or the stability of the aptamer. For example, oligonucleotides may be quickly degraded in bodily fluids by intracellular and extracellular enzymes such as endonucleases and exonucleases. In some cases, the aptamers described herein contain modified nucleotides to improve in vivo stability or to improve delivery characteristics.

Modifications of the aptamers contemplated in this disclosure include, without limitation, those which provide other chemical groups that incorporate additional charge, polarizability, hydrophobicity, hydrogen bonding, electrostatic interaction, and functionality such as increased resistance to nuclease degradation to the nucleic acid aptamer bases or to the nucleic acid aptamer as a whole. Modifications to generate oligonucleotide populations that are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, and unusual base-pairing combinations such as the isobases isocytidine and isoguanosine. Modifications can also include 3′ and 5′ modifications such as capping, e.g., addition of a 3′-3′-dT cap to increase exonuclease resistance.

In some cases, the aptamers described herein may be bound or conjugated to one or more molecules. Any number of molecules can be bound or conjugated to aptamers, non-limiting examples including antibodies, peptides, proteins, small molecules, gold nanoparticles, radiolabels, fluorescent labels, dyes, haptens (e.g., biotin), other aptamers, or nucleic acids (e.g., siRNA). In some cases, aptamers may be conjugated to molecules that increase the stability, the solubility or the bioavailability of the aptamer. Non-limiting examples include polyethylene glycol (PEG) polymers, carbohydrates and fatty acids. In some cases, molecules that improve the transport or delivery of the aptamer may be used, such as cell penetration peptides. Non-limiting examples of cell penetration peptides can include peptides derived from Tat, penetratin, polyarginine peptide Arg₈ sequence, Transportan, VP22 protein from Herpes Simplex Virus (HSV), antimicrobial peptides such as Buforin I and SynB, polyproline sweet arrow peptide molecules, Pep-1 and MPG.

In some instances, a polyethylene glycol (PEG) polymer chain is covalently bound to the aptamer, referred to herein as PEGylation. PEGylation may increase the half-life and stability of the aptamer in physiological conditions. In some cases, the PEG polymer is covalently bound to the 5′ end of the aptamer. In some cases, the PEG polymer is covalently bound to the 3′ end of the aptamer. In some cases, the PEG polymer is covalently attached to a specific nucleobase within the aptamer, such as the 5 position of a specific pyrimidine or the 8 position of a specific purine residue. The PEG polymer can have a molecular weight of, for example, 10 kDa, 20 kDa, 30 kDa, 40 kDa, 50 kDa, 60 kDa, 70 kDa, 80 kDa or greater. In some cases, the aptamers described herein are PEGylated. The PEG polymer can be branched, linear or any combination thereof wherein the total molecular weight is as described above.

### Sequencing Methods

In some cases, the aptamer is amplified and sequenced at various stages of the methods. Sequencing methods are well known in the art and may include Maxim-Gilbert, chain-termination or high-throughput systems. Alternatively, or additionally, the sequencing methods may comprise next generation sequencing, Helioscope™ single molecule sequencing, Nanopore DNA sequencing, Lynx Therapeutics' Massively Parallel Signature Sequencing (MPSS), 454 pyrosequencing, Single Molecule real time (RNAP) sequencing, Illumina (Solexa) sequencing, SOLiD sequencing, Ion Torrent™, Ion semiconductor sequencing, Single Molecule SMRT™ sequencing, Polony sequencing, DNA nanoball sequencing, VisiGen Biotechnologies approach, or a combination thereof. Alternatively, or additionally, the sequencing methods can comprise one or more sequencing platforms, including, but not limited to, Genome Analyzer IIx, HiSeq, NextSeq, and MiSeq offered by Illumina, Single Molecule Real Time (SMRT™) technology, such as the PacBio RS system offered by Pacific Biosciences (California) and the Solexa Sequencer, True Single Molecule Sequencing (tSMS™) technology such as the HeliScope™ Sequencer offered by Helicos Inc. (Cambridge, Mass.), nanopore-based sequencing platforms developed by Genia Technologies, Inc., and the Oxford Nanopore MinION.

### Kits

The methods of the present disclosure may be performed using a kit. The kit may comprise one or of the following: an aptamer library as described herein, one or more competitors, a solid substrate, a target, buffers, RNase inhibitor, DNase inhibitor, plates, beads, and magnetic beads.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. A Method for Improved Selection of Therapeutic Aptamers

### A. Protein Biotinylation

Target proteins are resuspended in PBS pH 7.2 to a final concentration of 5 μM. EZ-Link NHS-PEG4-Biotin (ThermoFisher Scientific) is prepared as a 20 mM stock according to manufacturer's instructions. 100 μl of 5 μM target protein is mixed with 1 μl of 20 mM NHS-PEG4-Biotin and incubated for 2 hours on ice. Unreacted biotin is removed by dialysis. The final volume of the biotinylated protein is brought to 1 mL in PBS pH 7.2 to yield a working stock of 500 nM biotinylated target protein. Biotin incorporation is determined with the Pierce Biotin Quantitation Kit (ThermoFisher Scientific).

### B. Aptamer Library Preparation

An aptamer bead library is resuspended in 10 mL Buffer B in a 15 mL conical tube (PBS pH 7.4 (10 mM phosphate buffer, 137.5 mM NaCl), 5.7 mM KCl, 1 mM MgCl₂, 1 mM CaCl₂, and 0.05% Tween-20). Tubes are centrifuged at 3000 rcf (swinging bucket rotor) for 10 minutes at room temperature. The supernatant is carefully removed by gentle aspiration, leaving ˜100 μl volume to wet beads. 3 mL of Buffer B is added to the aptamer bead library, incubated at 95° C. for 5 minutes, then allowed to cool for 30 minutes at room temperature. 7 mL of Buffer A (PBS pH 7.4 (10 mM phosphate buffer, 137.5 mM NaCl), 5.7 mM KCl, 1 mM MgCl₂, 1 mM CaCl₂, 0.2% BSA and 0.05% Tween-20) is added to the aptamer bead library and centrifuged at 3000 rcf (swinging bucket rotor) for 10 minutes at room temperature. Supernatant is carefully removed by gentle aspiration, leaving ˜100 μl volume to wet beads. Buffer A is added to a final volume of 1.8 mL and transferred to a 2 mL tube. Optional additional washes and centrifuges are performed to ensure transfer of all beads into the 2 mL tube.

### C. Coupling of Target Protein to Dynabeads M-280 Streptavidin Magnetic Beads

Dynabeads® M-280 Streptavidin beads (ThermoFisher Scientific, catalog #11205D) are resuspended well by gentle inversion. 250 μL M-280 beads are transferred into a 1.5 mL tube and tube is placed into a magnetic stand for ˜1 minute (until beads are fully captured), the supernatant is carefully removed by aspiration and discarded. 250 μL Buffer B is added to the M-280 beads and the beads are resuspended by inversion or gentle pipetting to wash the beads. Beads are captured using a magnetic stand, the supernatant is carefully removed by aspiration and discarded. The wash step is repeated 3 times with 250 μL Buffer B. The washed M-280 beads are resuspended in 100 μL of Buffer B. 300 μL of 500 nM biotinylated protein target is added to the washed M-280 beads. Beads are incubated for 30 minutes at room temperature with rotation. Target-coupled M-280 beads are captured using a magnetic stand, supernatant is carefully removed by aspiration and discarded. 200 μL Buffer B is added to the target-coupled M-280 beads and the beads are resuspended by inversion or gentle pipetting to wash the beads. Beads are captured a using magnetic stand, supernatant is carefully removed by aspiration, and discarded. The wash step is repeated for a total of 3 times with 200 μL Buffer B. The washed target-coupled M-280 beads are resuspended in 100 μL of Buffer A. The target-coupled M-280 beads are now ready for use in the Primary Positive Selection. The target-coupled beads are stored on ice until use.

### D. Negative Selection

This step will remove any aptamer library sequences with affinity towards the Dynabeads® M-280 Steptavidin magnetic beads. Briefly, M-280 beads are resuspended well by gentle inversion. 250 μL M-280 beads are transferred into a 1.5 mL tube, the tube is placed into a magnetic stand for ˜1 minute (until beads are fully captured by magnet), and the supernatant is carefully removed by aspiration and discarded. 500 μL Buffer A is added to the M-280 beads and the beads are resuspended by inversion or gentle pipetting to wash the beads. The beads are captured using a magnetic stand, and the supernatant is carefully removed by aspiration and discarded. The wash steps are repeated for a total of 3 times with 500 μL Buffer A. The washed M-280 beads are resuspended in 50 μL Buffer A.

Next, the washed M-280 beads from above are added to 1.8 mL of the prepared aptamer library beads and incubated for 1 hour at 37° C. with rotation. The M-280 beads are captured using a magnetic stand. The supernatant is carefully removed by aspiration and transferred (containing unbound Aptamer Library Beads) to a fresh 15 mL conical tube. 500 μL Buffer A is added to the M-280 beads and the beads are resuspended by inversion or gentle pipetting to wash beads. Beads are captured using a magnetic stand. The supernatant is gently removed by aspiration, and combined with the unbound Aptamer Library Beads from above. The M-280 beads are again washed, captured, and the supernatant is collected for a total of 4 times, and all unbound Aptamer Library Beads are combined. 10 mL of Buffer A is added to the unbound Aptamer Library Beads and the beads are mixed by gentle inversion to wash the beads. The Aptamer Library Beads are now pelleted by centrifugation at 3000 rcf (swinging bucket rotor) for 10 minutes at room temperature. The supernatant is gently removed by aspiration and discarded. The wash step is repeated for a total of 3 times with 10 mL Buffer A. The Aptamer Library beads are resuspended in a total volume of 1.8 mL Buffer A and transferred to a fresh 2 mL tube. The Aptamer Library Beads are now ready for primary positive selection with target proteins.

### E. Primary Positive Selection

This step will capture any Aptamer Library Beads containing sequences with affinity towards the target protein. Briefly, the entire volume of target-coupled M-280 beads from above is combined with 1.8 mL prepared Aptamer Library Beads (i.e., after negative selection) from above and incubated for 90 minutes at 37° C. with rotation. Half of the Aptamer Library Beads/target-coupled M-280 beads are transferred to a fresh 1.5 mL tube. The Aptamer Library Beads/target-coupled M-280 beads are captured using a magnetic stand. The supernatant is carefully removed by aspiration and discarded. The remaining Aptamer Library Beads/target-coupled M-280 beads are combined with the captured M-280 beads from above and resuspended by inversion or gentle pipetting. The Aptamer Library Beads/target-coupled M-280 beads are captured using a magnetic stand and the supernatant is carefully removed by aspiration and discarded. The Aptamer Library Beads/target-coupled M-280 beads are washed 8 times in 1 mL 37° C. Buffer A, the beads are captured in a magnetic stand, and the supernatant is aspirated and discarded after each wash. The Aptamer Library Beads/target-coupled M-280 beads are next washed 2 times in 1 mL 37° C. Buffer B, the beads are captured in a magnetic stand, and the supernatant is aspirated and discarded after each wash. The supernatant from the final wash should be clear (i.e., no remaining unbound Aptamer Library Beads). The Aptamer Library Beads/target-coupled M-280 beads are resuspended in 50 μL Buffer B. The Aptamer Library has now been enriched for target-binding sequences.

F. Cleavage of Aptamers from Beads

This step will cleave selected aptamers from the Aptamer Library Beads for use in the subsequent Secondary De-enrichment step. Briefly, 50 μL 1 N NaOH (or equal volume) is added to the 50 μL of Aptamer Library Bead/target-coupled M-280 beads from the Primary Positive Selection step and incubated at 65° C. for 30 minutes. 40 μL 2 M Tris-Cl is added to neutralize reaction. The M-280 beads are captured using a magnetic stand and the supernatant is transferred to a fresh 1.5 mL tube. The total volume should be ˜140 μL.

Next, the cleaved aptamers are desalted. Two Zeba Spin Desalting Columns (Thermo Fisher Scientific) are each placed in a 1.5 mL tube, and centrifuged at 1500×g for 1 minute to remove storage buffer. 300 μL Buffer B is added to the top of the resin bed and columns are centrifuged at 1500×g for 1 minute. Buffer is discarded. The wash step is repeated for a total of 3 times with 300 μL Buffer B, and each column is transferred to a new 1.5 mL tube. Half of the volume of the cleaved library from above is added to each desalting column. Columns are centrifuged at 1500×g for 2 minutes to collect the sample and columns are discarded. The eluents from each spin column are pooled. This is the cleaved aptamer library enriched for sequences binding to each of the target proteins.

### G. Secondary De-Enrichment Selection

This step will de-enrich the aptamers isolated in the Positive Primary Selection step for sequences specific to the epitope bound by the predicate competitor. Incubation #1 is performed according to Table 1. Briefly, in 1.5 mL tubes, reactions 1 to 5 as listed in Table 1 are prepared in the order from left to right. The target and bioactive epitope binder (e.g., a clinically relevant drug such as a mAb, antibody fragment, single-chain antibody, peptide or small molecule) are incubated for 15 minutes at 37° C. prior to adding the cleaved pool to the reaction. The reactions are incubated at 37° C. for 1 hour.

Next, incubation #2 is performed per Table 1. Briefly, Dynabead® M-280 Streptavidin magnetic beads are gently resuspended by inversion and 50 μl of beads are transferred to a fresh 1.5 mL tube. The M-280 beads are washed 3 times with 500 μL of Buffer B. The washed beads are resuspended in 25 μL of Buffer B. According to Table 1, 5 IA of M-280 beads are added to tubes 2-5. Tubes 1-5 are incubated for 30 minutes at 37° C. with rotation. Aptamer library/target-coupled M-280 beads are captured using a magnetic stand. The supernatant is carefully removed by aspiration and discarded. 150 μL of 37° C. Buffer B is added to the beads and the M-280 beads are resuspended by inversion or gentle pipetting to wash the beads. The M-280 beads are again captured using a magnetic stand, and the supernatant is carefully removed by aspiration and discarded. The wash step is repeated for a total of 3 times with 150 μL 37° C. Buffer B. The M-280 bead pellets are resuspended in 100 μL Buffer B. Each tube now contains aptamers de-enriched for the target of interest.

TABLE 1
Secondary Positive Selection Scheme.

| | Incubation #1 | | | | | | | | |
|---|---|---|---|---|---|---|---|---|---|
| | | | | 500 nM | | 5 μM | | | |
| | | | | Biotin- | | Anti- | | | Incubation #2 |
| | | Selection | | Target | | Target | | Cleaved | Magnetic |
| Fraction/barcode | Conditions | Buffer | | Protein | | Competitor | | Pool | particles |
| 1 | Start control | 135 | μl | 0 | μ1 | 0 | μl | 15 μl | 0 |
| 2 | 100 nM Target; | 105 | μl | 30 | μl | 0 | μl | 15 μl | + |
| | No Comp | | | | | | | | |
| 3 | 100 nM Target; | 103.5 | μl | 30 | μl | 1.5 | μl | 15 μl | + |
| | Low Comp | | | | | | | | |
| 4 | 100 nM Target; | 90 | μl | 30 | μl | 15 | μl | 15 μl | + |
| | High Comp | | | | | | | | |
| 5 | Negative control | 135 | μl | 0 | μl | 0 | μl | 15 μl | + |

### H. PCR and Gel Analysis of Selected Aptamers

A polymerase chain reaction (PCR) is prepared for each target and control reaction from the Secondary Positive Selection plus a “No Template Control” (NTC) (6 rxns total) using the enriched Aptamer/M-280 beads from above as template. PCR reactions are as follows:

1X PCR Buffer
2.5 mM MgCl2
0.2 mM dNTPs
0.4 uM Forward Primer (the same forward primer is used for each
reaction)
10 ul enriched Aptamer/M-280 beads
0.4 uM matched reverse primer #1-5 per Table 1. (Reverse Primer #5
is used for the NTC)
1 unit Taq polymerase
ddH20
100 uL total volume

PCR is performed per the following cycle conditions: a) initial denaturation at 94° C. for 1 minute; b) 20 cycles of 94° C. for 30 seconds, 50° C. for 30 seconds, 72° C. for 1 minute; final extension at 72° C. for 3 minutes. 10 μl of reaction is removed after every 4 cycles beginning after cycle #8 to determine the appropriate number of cycles to generate a clean PCR product without over-amplification.

Aliquots from PCR cycles 8, 12, 16 and 20 are analyzed on a 10% TBE polyacrylamide gel. The expected PCR product is ˜75 bp. An appropriate PCR cycle number to yield a clean PCR product for each target and control reaction from Table 1 is selected. A final PCR reaction is performed using the PCR conditions from above and the appropriate PCR cycle numbers as determined above.

### I. Next Generation Sequencing

80 μl of each of the above 5 PCR reactions (per Table 1) are combined into 1 tube, being careful not to include any M-280 beads. PCR products are sequenced and the identity of the enriched aptamers is determined.

### J. Bioinformatics

Aptamers to the target epitope are identified by comparative sequence analysis as follows: Aptamers to the target epitope are increased in frequency in Condition 2 as compared to Conditions 1 and 5, and those specific to the target epitope are decreased in frequency in Conditions 3 and 4 as compared to Condition 2 (Table 1).

### Example 2. A Method for Improved Selection of Therapeutic Aptamers

### A. Protein Biotinylation

Target proteins are resuspended in PBS pH 7.2 to a final concentration of 5 μM. EZ-Link NHS-PEG4-Biotin (ThermoFisher Scientific) is prepared as a 20 mM stock according to manufacturer's instructions. 100 μl of 5 μM target protein is mixed with 1 μl of 20 mM NHS-PEG4-Biotin and incubated for 2 hours on ice. Unreacted biotin is removed by dialysis. The final volume of the biotinylated protein is brought to 1 mL in PBS pH 7.2 to yield a working stock of 500 nM biotinylated target protein. Biotin incorporation is determined with the Pierce Biotin Quantitation Kit (ThermoFisher Scientific).

### B. Aptamer Library Preparation

An aptamer bead library is resuspended in 10 mL Buffer B in a 15 mL conical tube (PBS pH 7.4 (10 mM phosphate buffer, 137.5 mM NaCl), 5.7 mM KCl, 1 mM MgCl₂, 1 mM CaCl₂, and 0.05% Tween-20). Tubes are centrifuged at 3000 rcf (swinging bucket rotor) for 10 minutes at room temperature. The supernatant is carefully removed by gentle aspiration, leaving ˜100 μL volume to wet beads. 3 mL of Buffer B is added to the aptamer bead library, incubated at 95° C. for 5 minutes, then allowed to cool for 30 minutes at room temperature. 7 mL of Buffer A (PBS pH 7.4 (10 mM phosphate buffer, 137.5 mM NaCl), 5.7 mM KCl, 1 mM MgCl₂, 1 mM CaCl₂, 0.2% BSA and 0.05% Tween-20) is added to the aptamer bead library and centrifuged at 3000 rcf (swinging bucket rotor) for 10 minutes at room temperature. Supernatant is carefully removed by gentle aspiration, leaving ˜100 μL volume to wet beads. Buffer A is added to a final volume of 1.8 mL and transferred to a 2 mL tube. Optional additional washes and centrifuges are performed to ensure transfer of all beads into the 2 mL tube.

### C. Coupling of Target Protein to Magnetic Beads

Dynabeads® M-280 Streptavidin magnetic beads are resuspended well by gentle inversion. 250 μl M-280 beads are transferred into a 1.5 mL tube and tubes are placed into a magnetic stand for ˜1 minute (until beads are fully captured). The supernatant is carefully removed by aspiration and discarded. 250 μL Buffer B is added to the M-280 beads and the beads are resuspended by inversion or gentle pipetting to wash the beads. The beads are captured using a magnetic stand, and the supernatant is carefully removed by aspiration and discarded. The wash step is repeated for a total of 3 times with 250 μl Buffer B. The washed beads are resuspended in 100 μL of Buffer B. 300 μl of 500 nM biotinylated target protein is added to the washed M-280 beads and incubated 30 minutes at room temperature with rotation. Target-coupled M-280 beads are captured using a magnetic stand, and the supernatant is carefully removed by aspiration and discarded. 200 μL Buffer B is added to the target-coupled M-280 beads and the beads are resuspended by inversion or gentle pipetting to wash the beads. The beads are captured using a magnetic stand, and the supernatant is carefully removed by aspiration and discarded. The wash steps are repeated for a total of 3 times with 200 μL Buffer B. The washed target-coupled M-280 beads are resuspended in 100 μL of Buffer A. The target-coupled M-280 beads are now ready for use in the Primary Positive Selection.

### D. Negative Selection

This step will remove any aptamer library sequences with affinity towards the Dynabeads® M-280 Steptavidin magnetic beads. Briefly, M-280 beads are resuspended well by gentle inversion. 250 μl M-280 beads are transferred into a 1.5 mL tube, the tube is placed into a magnetic stand for ˜1 minute (until beads are fully captured by magnet), and the supernatant is carefully removed by aspiration and discarded. 500 μL Buffer A is added to the M-280 beads and the beads are resuspended by inversion or gentle pipetting to wash the beads. The beads are captured using a magnetic stand, and the supernatant is carefully removed by aspiration and discarded. The wash steps are repeated for a total of 3 times with 500 μl Buffer A. The washed M-280 beads are resuspended in 50 μL Buffer A.

Next, the washed M-280 beads from above are added to 1.8 mL of the prepared aptamer library beads and incubated for 1 hour at 37° C. with rotation. The M-280 beads are captured using a magnetic stand. The supernatant is carefully removed by aspiration and transferred (containing unbound Aptamer Library Beads) to a fresh 15 mL conical tube. 500 μL Buffer A is added to the M-280 beads and the beads are resuspended by inversion or gentle pipetting to wash beads. Beads are captured using a magnetic stand. The supernatant is gently removed by aspiration, and combined with the unbound Aptamer Library Beads from above. The M-280 beads are again washed, captured, and the supernatant is collected for a total of 4 times, and all unbound Aptamer Library Beads are combined. 10 mL of Buffer A is added to the unbound Aptamer Library Beads and the beads are mixed by gentle inversion to wash the beads. The Aptamer Library Beads are now pelleted by centrifugation at 3000 rcf (swinging bucket rotor) for 10 minutes at room temperature. The supernatant is gently removed by aspiration and discarded. The wash step is repeated for a total of 3 times with 10 mL Buffer A. The Aptamer Library beads are resuspended in a total volume of 1.8 mL Buffer A and transferred to a fresh 2 mL tube. The Aptamer Library Beads are now ready for primary positive selection with target proteins.

### E. Primary Positive Selection

This step will capture any Aptamer Library Beads containing sequences with affinity towards the target protein. Briefly, the entire volume of target-coupled M-280 beads from above is combined with 1.8 mL prepared Aptamer Library Beads (i.e., after negative selection) from above and incubated for 90 minutes at 37° C. with rotation. Half of the Aptamer Library Beads/target-coupled M-280 beads are transferred to a fresh 1.5 mL tube. The Aptamer Library Beads/target-coupled M-280 beads are captured using a magnetic stand. The supernatant is carefully removed by aspiration and discarded. The remaining Aptamer Library Beads/target-coupled M-280 beads are combined with the captured M-280 beads from above and resuspended by inversion or gentle pipetting. The Aptamer Library Beads/target-coupled M-280 beads are captured using a magnetic stand and the supernatant is carefully removed by aspiration and discarded. The Aptamer Library Beads/target-coupled M-280 beads are washed 8 times in 1 mL 37° C. Buffer A, the beads are captured in a magnetic stand, and the supernatant is aspirated and discarded after each wash. The Aptamer Library Beads/target-coupled M-280 beads are next washed 2 times in 1 mL 37° C. Buffer B, the beads are captured in a magnetic stand, and the supernatant is aspirated and discarded after each wash. The supernatant from the final wash should be clear (i.e., no remaining unbound Aptamer Library Beads). The Aptamer Library Beads/target-coupled M-280 beads are resuspended in 50 μL Buffer B. The Aptamer Library has now been enriched for target-binding sequences.

F. Cleavage of Aptamers from Beads

This step will cleave selected aptamers from the Aptamer Library Beads for use in the subsequent Secondary Positive De-enrichment step. Briefly, 50 μl 1 N NaOH (or equal volume) is added to the 50 μL of Aptamer Library Bead/target-coupled M-280 beads from the Primary Positive Selection step and incubated at 65° C. for 30 minutes. 40 μl 2 M Tris-Cl is added to neutralize reaction. The M-280 are captured beads using a magnetic stand and the supernatant is transferred to a fresh 1.5 mL tube. The total volume should be ˜140 μL.

Next, the cleaved aptamers are desalted. Two Zeba Spin Desalting Columns (Thermo Fisher Scientific) are each placed in a 1.5 mL tube, and centrifuged at 1500×g for 1 minute to remove storage buffer. 300 μL Buffer B is added to the top of the resin bed and columns are centrifuged at 1500×g for 1 minute. Buffer is discarded. The wash step is repeated for a total of 3 times with 300 μL Buffer B, and each column is transferred to a new 1.5 mL tube. Half of the volume of the cleaved library from above is added to each desalting column. Columns are centrifuged at 1500×g for 2 minutes to collect the sample and columns are discarded. The eluents from each spin column are pooled. This is the cleaved aptamer library enriched for sequences binding to each of the target proteins.

### G. Secondary Enrichment Selection

This step will enrich the aptamers isolated in the Positive Primary Selection step for sequences specific to the epitope bound by the predicate competitor. Incubation #1 is performed according to Table 2. Briefly, in 1.5 mL tubes, reactions 1 to 5 as listed in Table 2 are prepared. The reactions are incubated at 37° C. for 1 hour.

Next, incubation #2 is performed per Table 2. Briefly, Dynabeads® M-280 Streptavidin magnetic beads are gently resuspended by inversion and 50 μl of beads are transferred to a fresh 1.5 mL tube. The M-280 beads are washed 3 times with 500 μl of Buffer B. The washed beads are resuspended in 25 μL of Buffer B. According to Table 2, 5 μL of M-280 beads are added to tubes 2-5. Tubes 1-5 are incubated for 30 minutes at 37° C. with rotation. Aptamer library/target-coupled M-280 beads are captured using a magnetic stand. The supernatant is carefully removed by aspiration and discarded. 150 μL of 37° C. Buffer B is added and the M-280 beads are resuspended by inversion or gentle pipetting to wash the beads. The M-280 beads are again captured using a magnetic stand, and the supernatant is carefully removed by aspiration and discarded. The wash step is repeated for a total of 3 times with 150 μl 37° C. Buffer B. The M-280 bead pellets are resuspended in 100 μL Buffer A. Each tube now contains aptamers enriched for the target of interest.

TABLE 2
Secondary Positive Selection Scheme.

| | Incubation #1 | | | | |
|---|---|---|---|---|---|
| | | | 500 nM | | Incubation |
| | | | Biotin- | | #2 |
| Fraction/ | | Cleaved | Target | Selection | Magnetic |
| barcode | Conditions | Pool | Protein | Buffer | particles |
| 1 | Start | 15 μl | 0 μl | 135 μl | 0 |
| | control | | | | |
| 2 | 100 nM | 15 μl | 30 μl | 115 μl | + |
| | Target; No | | | | |
| | Comp | | | | |
| 3 | 100 nM | 15 μl | 30 μl | 115 μl | + |
| | Target; | | | | |
| | Low Comp | | | | |
| 4 | 100 nM | 15 μl | 30 μl | 115 μl | + |
| | Target; | | | | |
| | High | | | | |
| | Comp | | | | |
| 5 | Negative | 15 μl | 0 μl | 135 μl | + |
| | control | | | | |

Next, incubation #3 is prepared according to Table 3 to elute epitope-specific aptamers with a bioactive epitope binder (e.g., a clinically relevant drug such as a mAb, antibody fragment, single-chain antibody, peptide or small molecule). Briefly, to the 100 μL of enriched aptamers from above, reactions 1-5 are prepared per Table 3 below and incubated at 37° C. for 1 hour. Aptamer library/target-coupled M-280 beads are captured using a magnetic stand. For tubes 3 and 4, the supernatant is carefully removed and transferred to a new 1.5 mL tube, taking care not to transfer any M-280 beads. Each of tubes 3 and 4 contains aptamers enriched for the target epitope of interest. For tubes 1, 2, and 5, the M-280 bead pellets are resuspended by gentle pipetting. Each of tubes 1, 2, and 5 contains aptamers enriched to target of interest.

TABLE 3
Elution of Epitope-Specific Aptamers

| | | 5 μM Anti- | |
|---|---|---|---|
| Fraction/ | | Target | |
| Barcode | Conditions | Competitor | Selection Buffer B |
| 1 | Start Control | 0 μl | 50 μl |
| 2 | 100 nM Target; No | 0 μl | 50 μl |
| | Comp | | |
| 3 | 100 nM Target; Low | 1.5 μl | 48.5 μl |
| | Comp | | |
| 4 | 100 nM Target; High | 15 μl | 35 μl |
| | Comp | | |
| 5 | Negative Control | 0 μl | 50 μl |

### H. PCR and Gel Analysis of Selected Aptamers

A polymerase chain reaction (PCR) is prepared for each target and control reaction from the Secondary Positive Selection plus a “No Template Control” (NTC) (6 rxns total) using the enriched Aptamer/M-280 beads from above as template. PCR reactions are as follows:

1X PCR Buffer
2.5 mM MgCl2
0.2 mM dNTPs
0.4 uM Forward Primer (the same forward primer is used
for each reaction)
5 ul enriched Aptamer/M-280 beads
0.4 uM matched reverse primer #1-7 per Table 2.
(Reverse Primer #5 is used for the NTC)
1 unit Taq polymerase
ddH20
100 uL total volume

PCR is performed per the following cycle conditions: a) initial denaturation at 94° C. for 1 minute; b) 20 cycles of 94° C. for 30 seconds, 50° C. for 30 seconds, 72° C. for 1 minute; final extension at 72° C. for 3 minutes. 10 μl of reaction is removed after every 4 cycles beginning after cycle #8 to determine the appropriate number of cycles to generate a clean PCR product without over-amplification.

Aliquots from PCR cycles 8, 12, 16 and 20 are analyzed on a 10% TBE polyacrylamide gel. The expected PCR product is ˜75 bp. An appropriate PCR cycle number to yield a clean PCR product for each target and control reaction from Table 2 is selected. A final PCR reaction is performed using the PCR conditions from above and the appropriate PCR cycle numbers as determined above.

### I. Next Generation Sequencing

80 μl of each of the above 5 PCR reactions (per Table 2) are combined into 1 tube, being careful not to include any M-280 beads. PCR products are sequenced and the identity of the enriched aptamers is determined.

Aptamers to the target epitope are identified by comparative sequence analysis as follows: Aptamers to the target epitope are increased in frequency in Condition 2 as compared to Conditions 1 and 5, and those specific to the target epitope are further increased in frequency in Conditions 3 and 4 as compared to Condition 2 (Table 3).

### Example 3. Identification of Base-Modified Aptamers to the VEGF Receptor Binding Domain Using a De-Enrichment Method According to the Disclosure

A desirable mechanism of action for inhibitors of VEGF may be to bind to the receptor binding domain (RBD) in such a manner as to block the interaction of VEGF with its cognate receptors. Several inhibitors of VEGF employ this mechanism of action, including a monoclonal antibody to the RBD of VEGF having an amino acid sequence of heavy chain variable region of: EVQLVESGGGLVQPGGSLRLSCAASGYTFTNYGMNWVRQAPGKGLEWVGWINTYT GEPTYAADFKRRFTFSLDTSKSTAYLQMNSLRAEDTAVYYCAKYPHYYGS SHWYFD VWGQGTLVTVSSASTKGPSVFPLAPSSKSTSGGTAALGCLVKDYFPEPVTVSWNSGA LTSGVHTFPAVLQS SGLYSLSSVVTVPS SSLGTQTYICNVNHKPSNTKVDKKVEPKSC DKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIE KTISKAKGQPREPQVYTLPPSREEMTKNQVSLTCLVKGFYPSDIAVEWESNGQPENN YKTTPPVLDSDGSFFLYSKLTVDKSRWQQGNVFSCSVMHEALHNHYTQKSLSLSPG K (SEQ ID NO:3) and an amino acid sequence of light chain variable region of:

(SEQ ID NO: 4)
DIQMTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;

ranibizumab; and the VEGF receptor decoy, aflibercept. In addition to directly blocking the interaction between VEGF and its cognate receptors, inhibitors that bind the RBD of VEGF recognize all isoforms of VEGF, including VEGF₁₁₀, VEGF₁₂₁, VEGF₁₆₅ and VEGF₁₈₉, the latter two of which also contain a heparin binding domain. Numerous aptamer selections to VEGF have been conducted, which have demonstrated the heparin binding domain is the dominant epitope for aptamer selection. While the heparin binding domain is the dominant epitope against which aptamers to VEGF are generally isolated, it is assumed that aptamers are also selected to other epitopes of VEGF, albeit at a lower frequency within the aptamer libraries generated against VEGF. Therefore, to isolate aptamers to the RBD of VEGF, a selection strategy may need to be employed which enables the identification of aptamers to this domain of VEGF. In this example, a therapeutically relevant mAb to the RBD of VEGF having an amino acid sequence of heavy chain variable region according to SEQ ID NO:3 and of light chain variable region according to SEQ ID NO:4 was used in a de-enrichment protocol according to methods described herein to generate VEGF RBD aptamers.

### A. Preparation of Bead-Immobilized, Base-Modified Aptamer Libraries

Bead-immobilized, base-modified libraries for selection of aptamers to VEGF were constructed as follows. Briefly, polystyrene beads were used to synthesize bead-based library designs. For each library, synthesis was performed on four separate columns with a pool and split step after every second base to create a random region of fifteen two-base blocks based on a software-generated design. The two-base block library design enables a means to identify sites of incorporation of base-modified residues during analysis of the resultant aptamer sequence data. 5-Position-modified deoxyuridine residues were randomly scattered in the random region. This allows for library sequences that have from zero to twelve modifications. The three modifications used in this example (indoles, phenols and primary amines) were introduced with modified nucleoside phosphoramidites during library synthesis.

B. Preparation of Bead Immobilized Human VEGF₁₂₁ and VEGF₁₆₅.

Biotinylated recombinant human VEGF₁₆₅ was reconstituted at 100 μg/mL in deionized water. A 100 μg/mL solution was calculated to be 5.3 μM using the calculated monomer molecular weight of 19 kDa. Biotinylated recombinant human VEGF₁₂₁ was reconstituted at 100 μg/mL in deionized water. A 100 μg/mL solution was calculated to be 6 uM using the monomer molecular weight of 16.7 kDa.

Prior to the selection of aptamers, 250 pmoles each of VEGF₁₂₁ and VEGF₁₆₅ were coupled to Dynabeads M-280 Streptavidin Beads. M-280 beads were washed three times in 250 μl buffer B (10 mM phosphate buffer pH 7.4, 137.5 mM NaCl, 5.7 mM KCl, 1 mM MgCl₂, 1 mM CaCl₂, and 0.05% Tween) and resuspended in 100 μl buffer B, and then 47.2 μL of 5.3 μM VEGF₁₆₅ (250 pmoles) and 41.7 μl of 6.0 μM VEGF₁₂₁ (250 pmoles) were added to washed M-280 beads, and the solution was incubated at room temperature with rotation for 30 minutes. The VEGF-coupled beads were then captured using a magnetic stand, washed three times by gentle inversion with 200 μl buffer B, and resuspended in 100 μl of selection buffer A (10 mM phosphate buffer pH 7.4, 137.5 mM NaCl, 5.7 mM KCl, 1 mM MgCl₂, 1 mM CaCl₂, 0.2% BSA and 0.05% Tween).

### C. Aptamer Library Preparation and Negative Selection Against M-280 Beads.

A bead-coupled aptamer library was resuspended in 10 mL of buffer B, and washed by centrifugation at 3,000 rcf for 10 minutes, and the supernatant removed. The aptamer library was then resuspended in 3 mL of buffer B, heated at 95° C. for 5 minutes, and then cooled for 30 minutes at room temperature to renature the bead-immobilized aptamer library. The renatured aptamer library was then washed by adding 7 mL of buffer B, followed by centrifugation as before and resuspended in 1.8 mL buffer A. A 250 μl aliquot of non-VEGF coupled M-280 beads was washed three times with 500 μL buffer A, resuspended in final volume of 50 μl buffer A, and transferred to the tube containing the aptamer library. The aptamer library and non-VEGF coupled beads were incubated for 1 hour at 37° C. with rotation to allow any aptamers with affinity to the M-280 beads to bind to the M-280 beads. Following this incubation, the M-280 beads and any associated bead-immobilized aptamer library were collected on the magnetic stand, and the supernatant containing unbound aptamer library was removed and transferred to a fresh tube. The M-280 beads were gently washed four times with 500 μL of buffer A, and the supernatants from each wash were combined with the prior supernatant to generate a pool of aptamer library beads, pre-cleared of those with affinity to the M-280 streptavidin beads. The pre-cleared aptamer library was subsequently washed three times with 10 mL buffer A, and resuspended in 1.8 mL buffer A prior to use in selection of aptamers to the RBD of VEGF.

### D. Isolation of Aptamers to VEGF.

To identify aptamers to VEGF, the 100 μl of M-280 immobilized VEGF₁₂₁ and VEGF₁₆₅ was added to the pre-cleared aptamer library, and incubated for 90 minutes at 37° C. with rotation to enable binding of aptamers with affinity for VEGF to the M-280 bead coupled VEGF. Following the incubation, aptamers bound to VEGF were isolated by collection of the aptamer/VEGF-coupled M-280 beads complex using the magnetic stand, and the supernatant discarded. The aptamer/VEGF-coupled M-280 beads were then washed eight times with 1 mL of buffer A, followed by two times with 1 mL buffer B, with all wash buffers having been pre-warmed to 37° C. The aptamer/VEGF-coupled M-280 beads, now enriched for aptamers to VEGF, were then resuspended in 50 μL of buffer B.

Aptamers enriched to VEGF were then cleaved from beads by addition of an equal volume of 1 N NaOH and incubated at 65° C. for 30 minutes, followed by neutralization of the solution with 2 M Tris-Cl at a volume equivalent to 80% of the cleavage reaction. The aptamers to VEGF cleaved from the aptamer library beads were then desalted into selection buffer B.

### E. De-Enrichment of Aptamers Specific for the RBD of VEGF.

To identify aptamers specific to the RBD of VEGF, a second selection step was conducted using a clone of a therapeutically relevant mAb to the RBD of VEGF having an amino acid sequence of heavy chain variable region according to SEQ ID NO:3 and of light chain variable region according to SEQ ID NO:4 (anti-VEGF mAb). Binding reactions, including the Start and Negative control reactions, were prepared according to Table 4, with the order of addition as listed left to right in the table. VEGF isoforms and anti-VEGF mAb were incubated for 15 minutes at 37° C. prior to adding the cleaved pool to the reaction. Following addition of the cleaved aptamer pool to VEGF, the reactions listed in Table 4 were incubated at 37° C. for 1 hour with rotation. Aptamers were isolated by addition of 5 μL of M-280 beads, to reactions 2-7 as listed for Incubation #2 in Table 4, followed by incubation for 30 minutes at 37° C. Magnetic beads containing VEGF/aptamer complexes were subsequently captured with a magnetic stand and washed three times with 150 μL of buffer B pre-warmed to 37° C., and resuspended in 100 μL buffer B to generate aptamer pools de-enriched for aptamers to the RBD of VEGF.

TABLE 4
Secondary Positive Selection Scheme

| | Incubation #1 | | | | | | | | |
|---|---|---|---|---|---|---|---|---|---|
| | | | | | | 6.6 μM | | | Incubation #2 |
| | | Selection | | | | Anti-VEGF | | Cleaved | Magnetic |
| Fraction/barcode | Conditions | Buffer | | VEGF | | mAb | | Pool | particles |
| 1 | Start control | 135 | μl | 0 | μl | 0 | μl | 15 μl | 0 |
| 2 | VEGF₁₆₅; No mAb | 132.2 | μl | 2.8 | μl | 0 | μl | 15 μl | + |
| 3 | VEGF₁₆₅; Low mAb | 131.1 | μl | 2.8 | μl | 1.1 | μl | 15 μl | + |
| 4 | VEGF₁₆₅; High mAb | 120.8 | μl | 2.8 | μl | 11.4 | μl | 15 μl | + |
| 5 | VEGF₁₂₁; No mAb | 132.5 | μl | 2.5 | μl | 0 | μl | 15 μl | + |
| 6 | VEGF₁₂₁; High mAb | 121.1 | μl | 2.5 | μl | 11.4 | μl | 15 μl | + |
| 7 | Negative control | 135 | μl | 0 | μl | 0 | μl | 15 μl | + |

Note:
Final target protein concentration 100 nM; Low antibody concentration 50 nM; High antibody concentration 500 nM

Aptamers to the RBD of VEGF were identified by comparative sequence analysis as follows: Aptamers to VEGF₁₆₅ were increased in frequency in Condition 2 as compared to Conditions 1 and 7, and those specific to the RBD were reduced in frequency in Conditions 3 and 4 as compared to Condition 2 (Table 4). Similarly, aptamers to VEGF₁₂₁ were increased in Condition 5 as compared to Conditions 1 and 7, and those specific to the RBD were reduced in frequency in Conditions 6 as compared to Condition 5 (Table 4).

### F. Preparation of Isolated Aptamer Pools for Sequencing.

A PCR reaction was prepared for the VEGF aptamer pools as well as the start and negative control reactions by combining 5 μL of the isolated aptamers or control pools as template for each of 5×20 μl PCR reactions containing 1×PCR buffer, 2.5 mM MgCl₂, 0.2 mM dNTPs, 0.4 μM forward primer and 0.4 μM of reverse primer, with each set of PCR reactions containing a unique reverse primer containing a 6-nucleotide index for next generation sequencing, and 1 unit Taq polymerase. PCR reactions were run using an initial denaturation at 94° C. for 1 minute, followed by cycles of 94° C. for 30 seconds; 50° C. for 30 seconds; 72° C. for 1 minute, with a final extension of 72° C. for 3 minutes. The appropriate number of PCR cycles for each condition was determined in initial pilot PCR reactions. PCR products were subsequently purified using a Qiagen MinElute PCR Purification Kit, and subjected to next generation sequencing.

Sequences obtained from the selection strategy were analyzed as follows. Briefly, sites of base-modifications were restored to the individual sequences based on the two-base block synthetic codes and the design of the library. Frequencies for each sequence for each condition were determined, and normalized across each condition, and those sequences with approximately 2× or greater enrichment in fraction 2 as compared to 1 or 7 were considered VEGF₁₆₅ aptamers. Subsequently, the frequency of such sequences that met this criterion in fraction 2 were compared to fractions 3 and 4, and sequences whose frequencies decreased in fraction 3 and/or 4 were considered RBD aptamers. Similarly, those sequences with approximately 2× or greater enrichment in fraction 5 as compared to 1 or 7 were considered VEGF₁₂₁ aptamers. Subsequently the frequency of such sequences that met this criterion in fraction 5 were compared to fraction 6, and sequences whose frequency decreased in fraction 6 were considered RBD aptamers.

As shown in Table 5, this improved selection process led to the identification of aptamers that met the analysis criteria outlined above, and are therefore presumed to be specific to the RBD of VEGF.

TABLE 5
Aptamers to VEGF RBD Identified by De-enrichment
Methods to VEGF₁₆₅

| | Frequency in Condition | | | | |
|---|---|---|---|---|---|
| Aptamer # | 2 | 3 | 4 | 1 | 7 |
| 025 | 22468 | 699 | 474 | 139 | 806 |
| 026 | 9078 | 81 | 67 | 81 | 144 |
| 027 | 4771 | 364 | 147 | 53 | 828 |
| 028 | 3914 | 155 | 99 | 24 | 54 |
| 029 | 3229 | 270 | 129 | 30 | 108 |
| 030 | 2365 | 124 | 70 | 13 | 50 |
| 031 | 2268 | 124 | 73 | 22 | 41 |
| 032 | 2265 | 65 | 28 | 15 | 59 |
| 033 | 1820 | 68 | 49 | 8 | 27 |
| 034 | 1289 | 78 | 52 | 15 | 23 |
| 035 | 1208 | 286 | 163 | 17 | 842 |
| 036 | 1203 | 44 | 34 | 5 | 0 |
| 037 | 1136 | 305 | 197 | 23 | 275 |
| 038 | 875 | 6 | 21 | 5 | 0 |
| 039 | 835 | 174 | 29 | 9 | 27 |
| 040 | 815 | 31 | 9 | 11 | 0 |
| 041 | 806 | 457 | 244 | 23 | 108 |
| 042 | 791 | 367 | 192 | 17 | 81 |
| 043 | 774 | 420 | 202 | 23 | 104 |
| 044 | 762 | 6 | 5 | 5 | 0 |

As shown in Table 6, this improved selection process led to the identification of aptamers that met the analysis criteria outlined above, and are therefore presumed to be specific to the RBD of VEGF.

TABLE 6
Aptamers to VEGF RBD Identified by De-enrichment
Methods to VEGF₁₂₁

Frequency in Condition

| Compound # | 5 | 6 | 1 | 7 |
|---|---|---|---|---|
| 045 | 4770 | 28 | 42 | 86 |
| 046 | 2798 | 30 | 33 | 27 |
| 047 | 1698 | 26 | 29 | 266 |
| 048 | 967 | 505 | 541 | 662 |
| 049 | 801 | 4 | 0 | 0 |
| 050 | 712 | 0 | 9 | 63 |
| 051 | 700 | 0 | 2 | 0 |

### Example 4. Identification of Base-Modified Aptamers to the VEGF Receptor Binding Domain Using a Positive Enrichment Method According to the Disclosure

In this example, a therapeutically relevant mAb to the RBD of VEGF having an amino acid sequence of heavy chain variable region according to SEQ ID NO:3 and of light chain variable region according to SEQ ID NO:4 (Anti-VEGF mAb) was used in a positive enrichment protocol according to the methods described herein to generate VEGF RBD aptamers.

Briefly, preparation of bead immobilized recombinant VEGF, aptamer library preparation, negative selection, and selection of aptamers that bind to VEGF were essentially as described in Example 3 (see Example 3, A-D).

### E. Positive Enrichment of Aptamers Specific for the RBD of VEGF.

To identify aptamers specific to the RBD of VEGF, a second selection step was conducted using anti-VEGF mAb. Binding reactions, including the Start and Negative control reactions, were prepared according to Table 7, and were incubated at 37° C. for 1 hour with rotation. Aptamers were isolated by addition of 5 μL of M-280 beads, to reactions 2-7 as listed for Incubation #2 in Table 7, followed by incubation for 30 minutes at 37° C. Magnetic beads containing VEGF/aptamer complexes were subsequently captured with a magnetic stand and washed three times with 150 μL of buffer B pre-warmed to 37° C., and resuspended in 100 μL buffer B to generate aptamer pools further enriched for aptamers to VEGF.

TABLE 7
Secondary Positive Selection Scheme

| | | | | | Incubation |
|---|---|---|---|---|---|
| | Incubation #1 | | | | #2 |
| Fraction/ | | Cleaved | | Selection | Magnetic |
| barcode | Conditions | Pool | VEGF | Buffer B | particles |
| 1 | Start | 15 μl | 0 μl | 135 μl | 0 |
| | control | | | | |
| 2 | VEGF₁₆₅ | 15 μl | 2.8 μl | 132.2 μl | + |
| 3 | VEGF₁₆₅ | 15 μl | 2.8 μl | 132.2 μl | + |
| 4 | VEGF₁₆₅ | 15 μl | 2.8 μl | 132.2 μl | + |
| 5 | VEGF₁₂₁ | 15 μl | 2.5 μl | 132.5 μl | + |
| 6 | VEGF₁₂₁ | 15 μl | 2.5 μl | 132.5 μl | + |
| 7 | Negative | 15 μl | 0 μl | 135 μl | + |
| | Control | | | | |

Note:
Final target protein concentration is 100 nM

To identify aptamers to the RBD of VEGF, to the 100 μl of aptamers enriched to VEGF per Table 7 above, reactions to specifically elute aptamers to the RBD of VEGF were prepared per Table 8, and incubated at 37° C. for 2 hr. Following this incubation, aptamer library/VEGF-coupled M-280 beads were captured using the magnetic stand. For conditions 3, 4 and 6, aptamers eluted by anti-VEGF mAb were isolated by carefully removing the supernatant and transferring to a new tube, taking care not to transfer any M-280 beads. For conditions 1, 2, 5 and 7, the tubes were removed from the magnet and the M-280 bead pellets were resuspended by gentle pipetting.

TABLE 8
Elution of Epitope-Specific Aptamers

| Fraction/ | | 6.6 μM anti- | Selection |
|---|---|---|---|
| Barcode | Conditions | VEGF mAb | Buffer B |
| 1 | Start Control | 0 μl | 50 μl |
| 2 | VEGF₁₆₅; No mAb | 0 μl | 50 μl |
| 3 | VEGF¹⁶⁵; Low mAb | 1.1 μl | 48.9 μl |
| 4 | VEGF₁₆₅; High mAb | 11.4 μl | 38.6 μl |
| 5 | VEGF₁₂₁; No mAb | 0 μl | 50 μl |
| 6 | VEGF₁₂₁; High mAb | 11.4 μl | 38.6 μl |
| 7 | Negative Control | 0 μl | 50 μl |

Note:
Low mAb concentration is 50 nM;
High mAb concentration is 500 nM

Aptamers to the RBD of VEGF were identified by comparative sequence analysis as follows: Aptamers to VEGF₁₆₅ were increased in frequency in Condition 2 as compared to Conditions 1 and 7, and those specific to the RBD were further increased in frequency in Conditions 3 and 4 as compared to Condition 2 (Table 8). Similarly, aptamers to VEGF₁₂₁ were increased in Condition 5 as compared to Conditions 1 and 7, and those specific to the RBD were further increased in frequency in Conditions 6 as compared to Condition 5 (Table 8).

### F. Preparation of Isolated Aptamer Pools for Sequencing.

A PCR reaction was prepared for the VEGF aptamer pools as well as the start and negative control reactions by combining 5 μL of the isolated aptamers or control pools as template for each of 5×20 μl PCR reactions containing 1×PCR buffer, 2.5 mM MgCl₂, 0.2 mM dNTPs, 0.4 μM forward primer and 0.4 μM of reverse primer, with each set of PCR reactions containing a unique reverse primer containing a 6-nucleotide index for next generation sequencing, and 1 unit Taq polymerase. PCR reactions were run using an initial denaturation at 94° C. for 1 minute, followed by cycles of 94° C. for 30 seconds; 50° C. for 30 seconds; 72° C. for 1 minute, with a final extension of 72° C. for 3 minutes. The appropriate number of PCR cycles for each condition was determined initial pilot PCR reactions. PCR products were subsequently purified using a Qiagen MinElute PCR Purification Kit, and subjected to next generation sequencing.

Sequences obtained from the selection strategy were analyzed as follows. Briefly, sites of base-modifications were restored to the individual sequences based on the two-base block synthetic codes and the design of the library. Frequencies for each sequence for each condition were determined, and normalized across each condition, and those sequences with approximately 2× or greater enrichment in fraction 2 as compared to fraction 1 or 7 were identified as potential VEGF₁₆₅ aptamers. Subsequently, the frequency of such sequences that met this criterion in fraction 2 were compared to fractions 3 and 4, and sequences whose frequencies further increased in fraction 3 and/or 4 were considered RBD aptamers. Similarly, those sequences with approximately 2× or greater enrichment in fraction 5 as compared to 1 or 7 were considered VEGF₁₂₁ aptamers. Subsequently the frequency of such sequences that met this criterion in fraction 5 were compared to fraction 6, and sequences whose frequency further increased in fraction 6 were considered RBD aptamers.

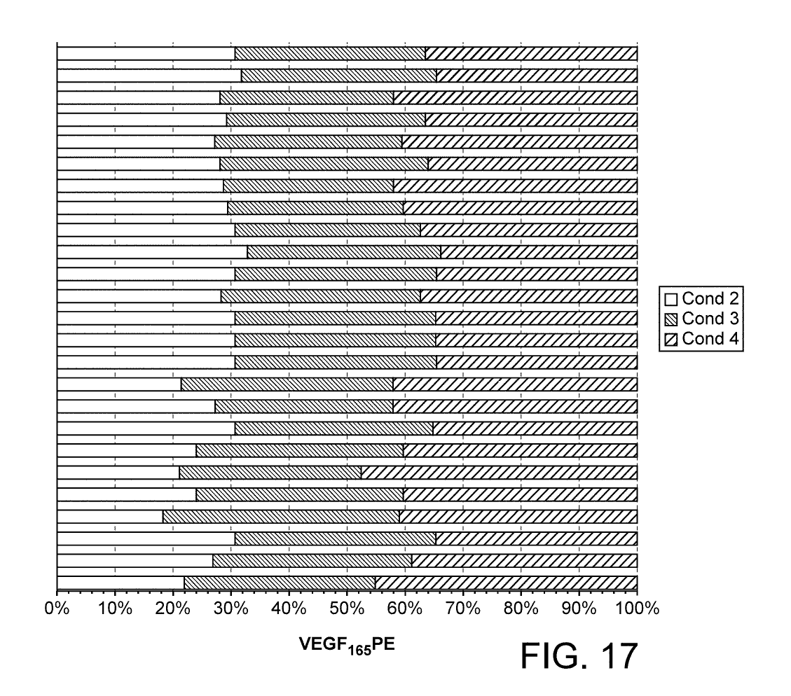

Aptamers enriched to VEGF₁₆₅ were identified as sequences enriched in Condition 2 as compared to 1 and 7 (Table 8), and these sequences were subsequently analyzed to identify those further enriched in Conditions 3 and 4 as Compared to Condition 2. As shown in FIG. 17, this improved selection process led to the identification of a number of aptamers for which the frequency increased upon elution with anti-VEGF mAb. These aptamers met the analysis criteria outlined above, and were therefore presumed to be specific to the RBD of VEGF

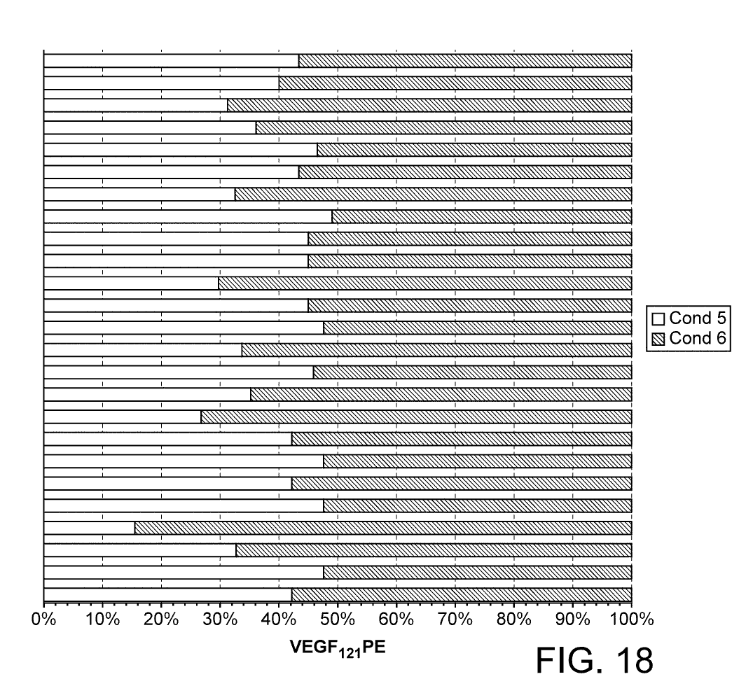

Aptamers enriched to VEGF₁₂₁ were identified as sequences enriched in Condition 5 as compared to 1 and 7 (Table 8), and these sequences were subsequently analyzed to identify those further enriched in Conditions 6 as Compared to 5. As shown in FIG. 18, this improved selection process led to the identification of a number of aptamers for which the frequency increased upon elution with anti-VEGF mAb. These aptamers met the analysis criteria outlined above, and were therefore presumed to be specific to the RBD of VEGF.

### Example 5. Identification of Base-Modified Aptamers to the Exosite of Complement Factor D (fD) Using a Method According to the Disclosure

A desirable mechanism for inhibition of fD activation C3bB to C3bBb may be to prevent the association of C3bB with fD, an interaction which occurs via the exosite of fD. Inhibitors of if) such as an anti-fD Fab having an amino acid sequence of heavy chain variable region according to SEQ ID NO:1 and of light chain variable region according to SEQ ID NO:2 (anti-fD Fab), inhibit fD activation of C3bB by binding to the exosite of fD and blocking its association with C3bB. The dominant epitope for aptamer generation to fD has not been established. In this example, anti-fD Fab was used in a positive enrichment and de-enrichment protocol in accordance with embodiments of the disclosure to generate aptamers to the exosite of fD.

### A. Preparation of Bead-Immobilized, Base-Modified Aptamer Libraries

Bead-immobilized, base-modified libraries for selection of aptamers to fD were constructed as follows. Briefly, polystyrene beads were used to synthesize bead-based library designs. For each library, synthesis was performed on four separate columns with a pool and split step after every second base to create a random region of fifteen two-base blocks based on a software-generated design. The two-base block library design enables a means to identify sites of incorporation of base-modified residues during analysis of the resultant aptamer sequence data. 5-Position-modified deoxyuridine residues were randomly scattered in the random region. This allows for library sequences that have from zero to twelve modifications. The three modifications used in this example (indoles, phenols and primary amines) were introduced with modified nucleoside phosphoramidites during library synthesis.

### B. Preparation of Bead Immobilized Human ID.

Human fD was reconstituted at 5 μM final concentration in PBS, pH 7.2, and 100 μl of fD was combined with 1 μL of 20 mM EZ-Link™ NHS-PEG4 Biotin and incubated for 2 hours on ice. Following this incubation, unreacted biotin was removed by dialysis into selection buffer B ((10 mM phosphate buffer pH 7.4, 137.5 mM NaCl), 5.7 mM KCl, 1 mM MgCl₂, 1 mM CaCl₂, and 0.05% Tween), the biotin incorporation was determined with a Biotin Quantitation Kit (Pierce), and then the biotinylated fD was diluted to 500 nM in selection buffer B.

Prior to the selection of aptamers, 500 pmoles of fD was coupled to Dynabeads® M-280 Streptavidin Beads. M-280 beads were washed three times in 250 μl buffer B and resuspended in 100 μL buffer B, and then 150 μL of 5.0 μM fD (500 pmoles) was added to washed M-280 beads, and the solution was incubated at room temperature with rotation for 30 minutes. The fD-coupled beads were then captured using a magnetic stand, washed three times by gentle inversion with 200 μl buffer B, and resuspended in 100 μl of selection buffer A ((10 mM phosphate buffer pH 7.4, 137.5 mM NaCl), 5.7 mM KCl, 1 mM MgCl₂, 1 mM CaCl₂, 0.2% BSA and 0.05% Tween).

### C. Aptamer Library Preparation and Negative Selection Against M-280 Beads.

The bead-coupled aptamer library was resuspended in 10 mL of buffer B, and washed by centrifugation at 3,000 rcf for 10 minutes, and the supernatant was removed. The aptamer library was then resuspended in 3 mL of buffer B, heated at 95° C. for 5 minutes, and then cooled for 30 minutes at room temperature to renature the bead-immobilized aptamer library. The renatured aptamer library was then washed by adding 7 mL of buffer B, followed by centrifugation as before and resuspended in 1.8 mL buffer A. A 250 μl aliquot of non-fD coupled M-280 beads was washed three times with 500 μL buffer A, resuspended in final volume of 50 μl buffer A, and transferred to the tube containing the aptamer library. The aptamer library and non-fD coupled beads were incubated for 1 hour at 37° C. with rotation to allow any aptamers with affinity to the M-280 beads to bind to the M-280 beads. Following this incubation, the M-280 beads and any associated bead-immobilized aptamer library were collected on the magnetic stand, and the supernatant containing unbound aptamer library was removed and transferred to a fresh tube. The M-280 beads were gently washed four times with 500 μL of buffer A, and the supernatants from each wash were combined with the prior supernatant to generate a pool of aptamer library beads, pre-cleared of those with affinity to the M-280 streptavidin beads. The pre-cleared aptamer library was subsequently washed three times with 10 mL buffer A, and resuspended in 1.8 mL buffer A prior to use in selection of aptamers to the exosite of fD.

### D. Isolation of Aptamers to fD.

To identify aptamers to fD, the 100 μl of M-280 immobilized fD was added to the pre-cleared aptamer library, and incubated for 90 minutes at 37° C. with rotation to enable binding of aptamers with affinity for fD to the M-280 bead coupled fD. Following the incubation, aptamers bound to fD were isolated by collection of the aptamer/fD-coupled M-280 beads complex using the magnetic stand, and the supernatant was discarded. The aptamer/fD-coupled M-280 beads were then washed eight times with 1 mL of buffer A, followed by two times with 1 mL buffer B, with all wash buffers having been pre-warmed to 37° C. The aptamer/fD-coupled M-280 beads, now enriched for aptamers to fD, were then resuspended in 50 μl of buffer B.

Aptamers enriched to fD were then cleaved from beads by addition of an equal volume of 1 N NaOH and incubation at 65° C. for 30 minutes, followed by neutralization of the solution with 2 M Tris-Cl at a volume equivalent to 80% of the cleavage reaction. The aptamers to fD cleaved from the aptamer library beads were then desalted into selection buffer B.

### E. Positive Enrichment of Aptamers Specific for the Exosite of fD.

To identify aptamers specific to the exosite of fD, a second selection step was conducted using anti-fD Fab. Binding reactions, including the Start and Negative control reactions, were prepared according to Table 9 and Table 11, and were incubated at 37° C. for 1 hour with rotation. Aptamers were isolated by addition of 5 μL of M-280 beads, to reactions 2-4 as listed for Incubation #2 in Table 9, followed by incubation for 30 minutes at 37° C. Magnetic beads containing fD/aptamer complexes were subsequently captured with a magnetic stand and washed three times with 150 μL of buffer B pre-warmed to 37° C., and resuspended in 100 μL buffer B to generate aptamer pools further enriched for aptamers to fD.

TABLE 9
Secondary Positive Selection Scheme

| | | | | | Incubation |
|---|---|---|---|---|---|
| | Incubation #1 | | | | #2 |
| Fraction/ | | Cleaved | | Selection | Magnetic |
| barcode | Conditions | Pool | fD | Buffer B | particles |
| 1 | Start | 15 μl | 0 μl | 135 μl | 0 |
| | control | | | | |
| 2 | fD | 15 μl | 3.75 μl | 131.25 μl | + |
| 3 | fD | 15 μl | 3.75 μl | 131.25 μl | + |
| 4 | fD | 15 μl | 3.75 μl | 131.25 μl | + |

Note:
Final target protein concentration is 125 nM

To identify aptamers to the exosite of fD, to the 100 μl of aptamers enriched to fD per Table 9 above, reactions to specifically elute aptamers to the exosite of fD were prepared per Table 10, and incubated at 37° C. for 2 hr. Following this incubation, aptamer library/fD-coupled M-280 beads were captured using the magnetic stand. For conditions 3 and 4, aptamers eluted by anti-fD Fab were isolated by carefully removing the supernatant and transferring to a new tube, taking care not to transfer any M-280 beads. For condition 1 and 2, the tubes were removed from the magnet and the M-280 bead pellets were resuspended by gentle pipetting.

TABLE 10
Elution of Epitope-Specific Aptamers

| Fraction/ | | 10 μM Anti-fD | Selection |
|---|---|---|---|
| Barcode | Conditions | Fab | Buffer B |
| 1 | Start Control | 0 μl | 50 μl |
| 2 | fD; No Fab | 0 μl | 50 μl |
| 3 | fD; Low Fab | 1.5 μl | 48.5 μl |
| 4 | fD; High Fab | 15 μl | 35 μl |

Note:
Low Fab concentration is 50 nM;
High Fab concentration is 500 nM

Aptamers to the exosite of fD were identified by comparative sequence analysis as follows. Aptamers to fD were increased in frequency in Condition 2 as compared to Conditions 1 and 7, and those specific to the exosite of fD were further increased in frequency in Conditions 3 and 4 as compared to Condition 2 (Table 10).

### F. De-Enrichment of Aptamers Specific for the Exosite of fD.

To identify aptamers specific to the exosite of fD, a second selection step was conducted using a clone of the Fab lamplizumab. Binding reactions, including the Start and Negative control reactions, were prepared according to Table 9 and Table 11, with the order of addition as listed left to right in Table 11. Factor D and anti-fD Fab were incubated for 15 minutes at 37° C. prior to adding the cleaved pool to the reaction. Following addition of the cleaved aptamer pool to fD, the reactions listed in Table 9 were incubated at 37° C. for 1 hour with rotation (for start control and selection to fD without anti-fD Fab). Aptamers were isolated by addition of 54 of M-280 beads, to reactions 5-7 as listed for Incubation #2 in Table 11, followed by incubation for 30 minutes at 37° C. Magnetic beads containing fD/aptamer complexes were subsequently captured with a magnetic stand and washed three times with 150 μL of buffer B pre-warmed to 37° C., and resuspended in 1004 buffer B to generate aptamer pools de-enriched for aptamers to the exosite of fD.

TABLE 11
Secondary Positive Selection Scheme

| | Incubation #1 | | | | | | | | |
|---|---|---|---|---|---|---|---|---|---|
| | | | | | | 10 μM | | | Incubation #2 |
| | | Selection | | | | Anti-fD | | Cleaved | Magnetic |
| Fraction/barcode | Conditions | Buffer | | fD | | Fab | | Pool | particles |
| 5 | fD; Low Fab | 129.75 | μl | 3.75 | μl | 1.5 | μl | 15 μl | + |
| 6 | fD; High Fab | 116.25 | μl | 3.75 | μl | 15 | μl | 15 μl | + |
| 7 | Negative control | 135 | μl | 0 | μl | 0 | μl | 15 μl | + |

Note:
Final target protein concentration 125 nM; Low Fab concentration 50 nM; High Fab concentration 500 nM

Aptamers to the exosite of fD were identified by comparative sequence analysis as follows. Aptamers to fD were increased in frequency in Condition 2 as compared to Conditions 1 and 7, and those specific to the exosite of fD were reduced in frequency in Conditions 6 and 7 as compared to Condition 2 (Table 11).

### G. Preparation of Isolated Aptamer Pools for Sequencing.

A PCR reaction was prepared for the fD aptamer pools as well as the start and negative control reactions by combining 5 μL of the isolated aptamers or control pools as template for each of 5×204 PCR reactions containing 1×PCR buffer, 2.5 mM MgCl₂, 0.2 mM dNTPs, 0.4 μM forward primer and 0.4 μM of reverse primer, with each set of PCR reactions containing a unique reverse primer containing a 6-nucleotide index for next generation sequencing, and 1 unit Taq polymerase. PCR reactions were run using an initial denaturation at 94° C. for 1 minute, followed by cycles of 94° C. for 30 seconds; 50° C. for 30 seconds; 72° C. for 1 minute, with a final extension of 72° C. for 3 minutes. The appropriate number of PCR cycles for each condition was determined in initial pilot PCR reactions. PCR products were subsequently purified using a Qiagen MinElute PCR Purification Kit, and subjected to next generation sequencing.

Sequences obtained from the selection strategy were analyzed as follows. Briefly, sites of base-modifications were restored to the individual sequences based on the two-base block synthetic codes and the design of the library. Frequencies for each sequence for each condition were determined, and normalized across each condition, and those sequences with approximately 2× or greater enrichment in fraction 2 as compared to fractions 1 or 7 were identified as potential fD aptamers.

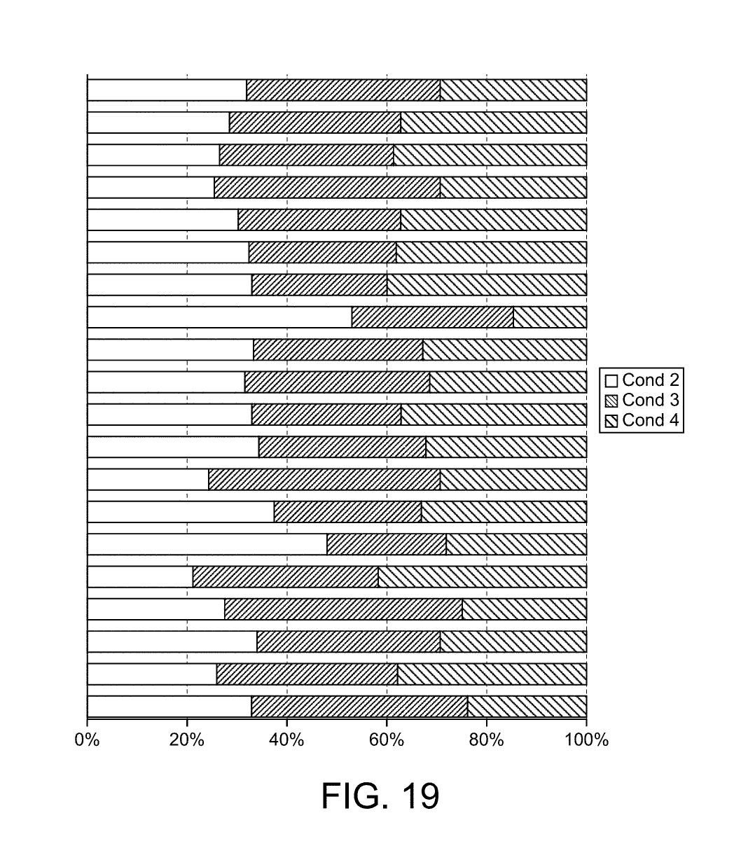

Aptamers enriched to fD by the positive enrichment protocol were identified as sequences enriched in Condition 2 as compared to 1 and 7 (Table 10), and these sequences were subsequently analyzed to identify those further enriched in Conditions 3 and 4 as Compared to Condition 2. As shown in FIG. 19, this improved selection process led to the identification of a number of aptamers for which the frequency increased upon elution with anti-fD Fab. These aptamers met the analysis criteria outlined above, and were therefore presumed to be specific for the exosite of fD.

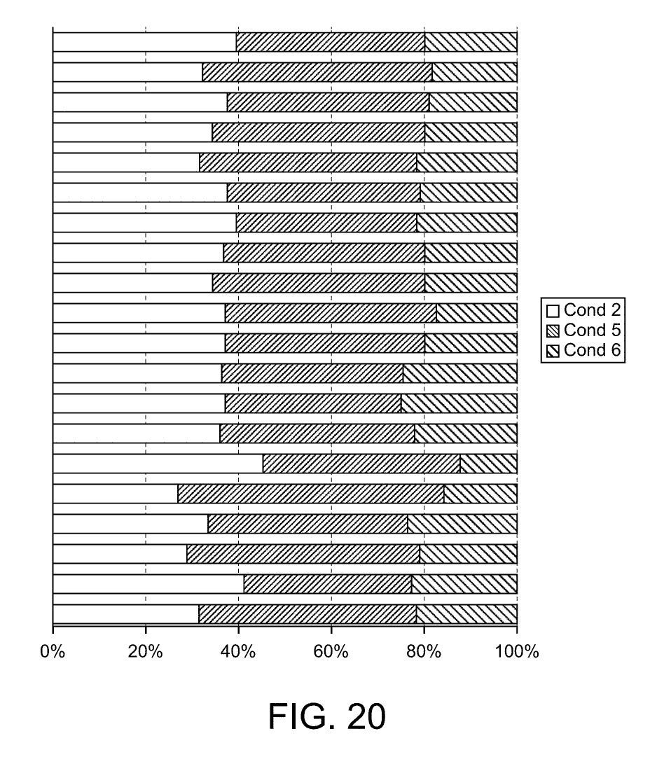

Additional aptamers enriched to the exosite of fD using the de-enrichment protocol were identified as sequences enriched in Condition 2 as compared to 1 and 7 (Table 10 and Table 11), and these sequences were subsequently analyzed to identify those for which the frequency decreased in Conditions 5 and 6 as Compared to Condition 2. As shown in FIG. 20, this improved selection process led to the identification of a number of aptamers for which the frequency decreased when the exosite of fD was masked with anti-fD Fab, particularly at the higher anti-fD Fab concentration used in condition 6. These aptamers met the analysis criteria outlined above, and were therefore presumed to be specific to the exosite of fD.

### Example 6. Identification of Modified RNA Aptamers to the Exosite of fD Using a Method According to the Disclosure

### A. Enrichment of Library for Factor D Aptamers in the Primary Selection

To identify aptamers to the exosite of fD, an initial primary selection to fD was performed to generate an aptamer library enriched to fD. The aptamer library was comprised of a 30-nucleotide random region flanked by constant regions containing a built-in stem region. For nuclease stability, the library was composed of 2′F G and 2′-O-methyl A/C/U. The purpose of the primary selection was to generate an aptamer library that was enriched in aptamers to fD, yet maintained an appropriate level of sequence diversity for use in the selection method.

The starting library was transcribed from a pool of ˜10¹⁴ dsDNA molecules. The dsDNA library was generated by primer extension using Klenow exo (−) DNA polymerase, a pool forward primer and synthetic ssDNA molecule encoding the library. The dsDNA was subsequently converted to 100% backbone modified RNA via transcription using a mixture of 2′F GTP, 2′-O-methyl ATP/CTP/UTP and a variant of T7 RNA polymerase bearing the mutations Y639L and H784A in buffer optimized to facilitate efficient transcription. Following transcription, RNAs were treated with DNAse to remove the template dsDNA and were purified.

The selection targeting fD was facilitated by the use of a His-tagged recombinant human fD protein and magnetic His capture beads. Briefly, various amounts of beads were washed three times with immobilization buffer (50 mM sodium phosphate, pH 8.0, 300 mM NaCl, 0.01% Tween-20) and were resuspended in 50 μL of immobilization buffer. fD, in immobilization buffer, was then added to the beads and incubated at room temperature for 30 minutes. The amount of target protein in the primary selection varied with the rounds (Table 12). The beads were washed 3 times with binding buffer SB1T (40 mM HEPES, pH 7.5, 125 mM NaCl, 5 mM KCl, 1 mM MgCl₂, 1 mM CaCl₂, 0.05% Tween-20) to remove any unbound protein and were then re-suspended in 50 μL SB1T buffer containing 1 μg/μl ssDNA and 0.1% BSA.

For the first round of selection, ˜3 nanomoles of the Round 0 RNA pool, ˜10″ sequences, was used. Prior to each round, the library was thermally equilibrated by heating at 80° C. for 5 minutes and cooled at room temperature for 15 minutes in the presence of a 1.5-fold molar excess of reverse primer to allow the library to refold and simultaneously block the 3′ end of the pool. Following renaturation, the final volume of the reaction was adjusted to 50 μL, in SB1T supplemented with 1 μg/ml ssDNA and 0.1% BSA.

For the first round, the library was added to the fD immobilized on beads and incubated at 37° C. for 1 hour with intermittent mixing. After an hour, the beads were washed using 3×1 ml SB1T buffer to remove unbound aptamers. For round 0, each wash step was incubated for 5 minutes. After washing, fD bound aptamers were eluted using 200 μL elution buffer (2M Guanidine-HCl in SB1T buffer) two times (total volume 400 μL). The eluted aptamers, in 400 μL of elution buffer, were precipitated by adding 40 μL 3M NaOAc, pH 5.2, 1 ml ethanol and 2 μl glycogen and incubating at −80° C. for 15 minutes. The recovered library was converted to DNA by reverse transcription using Super Script™ IV reverse transcriptase, and the ssDNA was subsequently amplified by PCR. The resulting dsDNA library was subsequently converted back into modified RNA via transcription as described above. DNased, purified RNA was used for subsequent rounds.

For subsequent rounds, the washing time and number of washes was varied as the selection progressed, the input RNA was kept fixed at 25 picomole, and the protein input varied (Table 12). After the first round, a negative selection step was included in all the subsequent rounds. For the negative selection, the pool was prepared as described before and first incubated with non-labelled beads for 1 hour at 37° C. in SB1T buffer. The beads were then spun down and the supernatant containing molecules that did not bind to the unlabeled beads, were incubated with fD labeled beads for an additional 1 hour at 37° C.

TABLE 12
Primary Selection details

| | | Target | | | | | |
|---|---|---|---|---|---|---|---|
| | Input library | protein | Binding | Washing | | | |
| Round | pmoles/conc | pmoles/conc | buffer | buffer | washes | #cycles | NGS |
| 0 | 1000 pm/40 μM | 40 pm/0.4 μM | SB1T | SB1T | 3 × 5 min | 22 | yes |
| 1 | 25 pm/1 μM | 40 pm/0.4 μM | SB1T | SB1T | 3 × 5 min | 22 | yes |
| 2 | 25 pm/1 μM | 40 pm/0.4 μM | SB1T | SB1T | 3 × 5 min | 20 | yes |
| 3 | 25 pm/1 μM | 4 pm/0.04 μM | SB1T | SB1T | 3 × 5 min | 18 | yes |
| 4 | 25 pm/1 μM | 8 pm/0.08 μM | SB1T | SB1T | 3 × 10 min | 18 | yes |
| 5 | 25 pm/1 μM | 8 pm/0.08 μM | SB1T | SB1T | 3 × 10 min | 16 | yes |
| 6 | 25 pm/1 μM | 4 pm/0.04 μM | HBSS | SB1T | 4 × 15 min | 14 | yes |
| 7 | 25 pm/1 μM | 4 pm/0.04 μM | HBSS | HBSS + SB1T | 4 × 15 min | 14 | yes |
| 8 | 25 pm/1 μM | 4 pm/0.04 μM | SB1T | SB1T | 4 × 15 min | 12 | yes |

### B. Assessing the Progress of Selection

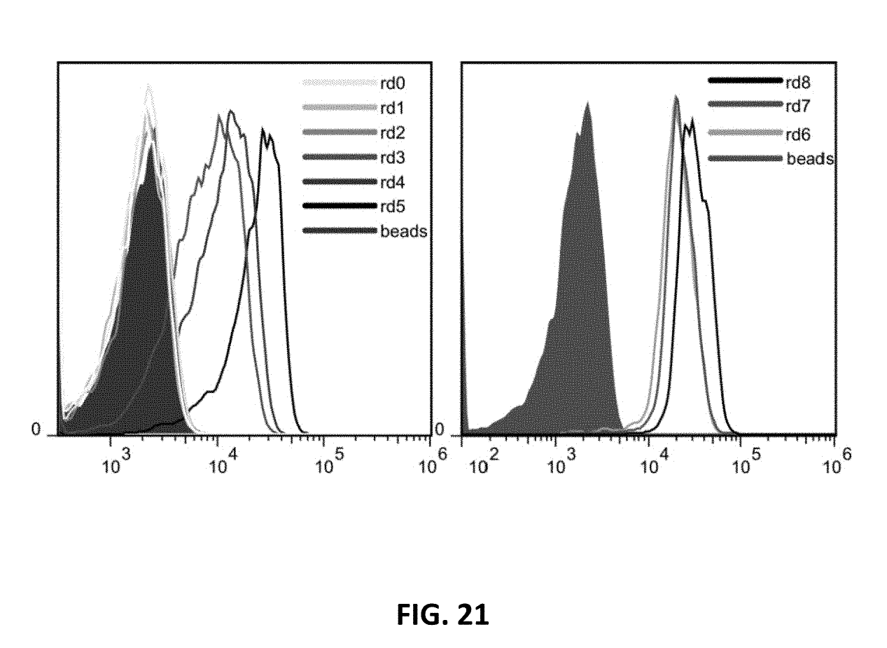

Flow cytometry was used to assess the progress of the selection. For these assays, RNA from each round was first hybridized with reverse complement oligonucleotide composed of 2′OMe RNA labeled with Dylight®650 (Dy650-N30S.R.OMe). Briefly, the library was combined with 1.5-fold molar excess of Dy650-N30S.R.OMe, was heated at 80° C. for 6 minutes and allowed to cool at room temperature for 15 minutes after which it was incubated with beads labelled with fD in SB1T buffer containing 0.1% BSA and 1 μg/μl ssDNA. Following incubation for 1 hour at 37° C., the beads were washed three times with SB1T, re-suspended in SB1T buffer and analyzed by flow cytometry. As shown in FIG. 21, an improvement in fluorescent signal with the progressing rounds was seen as early as Round 3. After Round 6, there was little change in the binding signal though Round 8. The apparent affinity of rounds 6, 7, and 8 for fD was also measured using flow cytometry-based assays and revealed K_{d}s in the range of 8-45 nM (FIG. 21; Table 13).

TABLE 13
Affinity constant of selected rounds and aptamers
generated in selection to ID

| Round | K_{d} (nM) |
|---|---|
| Rd 6 | 34.4 |
| Rd 7 | 45.1 |
| Rd 8 | 8.8 |

### C. Positive Enrichment of Aptamers Specific for the Exosite of fD.

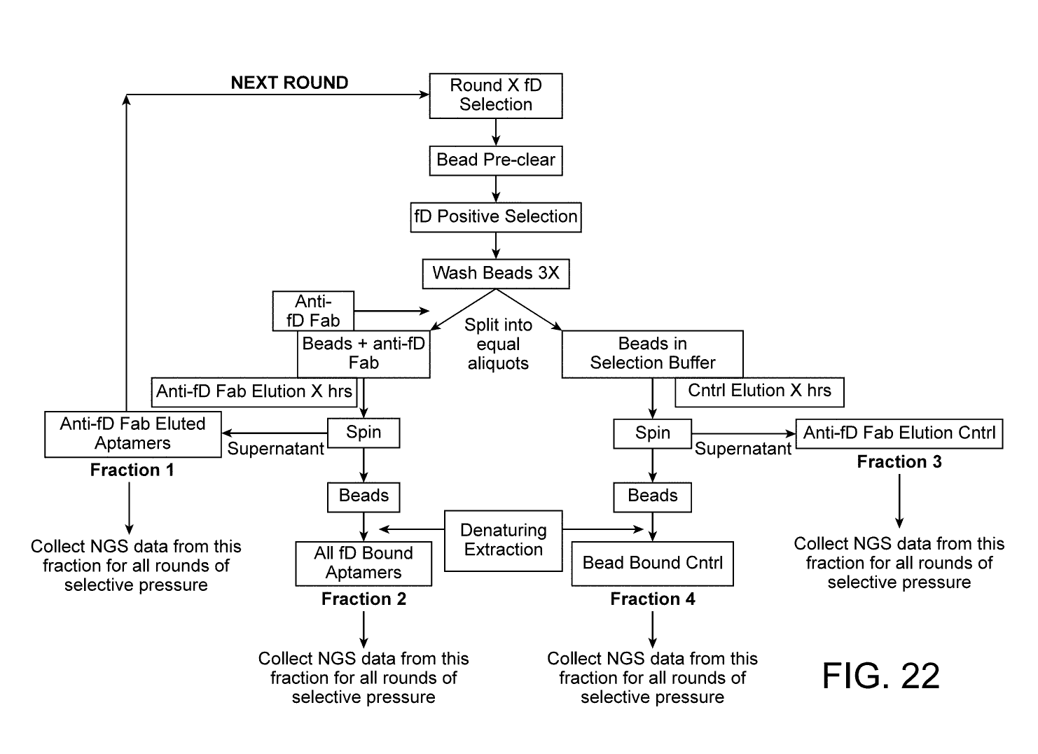

To identify modified RNA aptamers specific to the exosite of fD, a secondary selection was conducted using an anti-fD Fab having an amino acid sequence of heavy chain variable region according to SEQ ID NO:1 and of light chain variable region according to SEQ ID NO:2 (anti-fD Fab). Round 7 of the primary selection to fD was chosen to use in the selection scheme outlined in FIG. 22. Binding reactions were assembled as outlined in Table 14, with the initial primary selection reaction (Round 7) as the new input RNA. Briefly, the input RNA was renatured as described above, the negative selection was conducted as described above, and the resultant input RNA was added to immobilized fD at the concentrations listed in Table 14 and allowed to incubate for 1 hour at 37° C. with intermittent shaking. Input library and target concentration in Table 14 represent the concentration of each in the positive selection step, prior to washing and splitting of the binding reaction into aliquots with and without anti-fD Fab, while the input anti-fD Fab represents the final anti-fD Fab concentration in the anti-fD Fab elution arm of the selection scheme. Following this incubation, the immobilized fD beads were washed three times for 10 minutes each, and the reactions were split into an anti-fD Fab elution arm and a control elution arm. Aptamers were eluted with anti-fD Fab for 1 hour, the beads were pelleted by centrifugation, and the supernatant was collected to yield Fraction 1 (i.e., competitively eluted fraction). Aptamers that remained on the beads following elution with anti-fD Fab were recovered by guanidinium extraction as described above to yield Fraction 2. In the control elution arm, Fraction 3 represented aptamers that dissociated from the beads during the 1 hour mock elution, whereas Fraction 4 represented aptamers that were retained on the beads at the end of the mock elution. Fraction 1, aptamers eluted by anti-fD Fab, were processed for subsequent rounds of selections, whereas fractions 2-4 were collected and archived for comparative sequence analysis at each round of selection.

TABLE 14
Conditions for Positive Enrichment of Aptamers to
the Exosite of fD

| Round | Input Library | Target Protein | Input anti-fD Fab |
|---|---|---|---|
| 8 V | 25 pmole/250 nM | 4 pmole/40 nM | 100 nM |
| 9 V | 25 pmole/250 nM | 4 pmole/40 nM | 100 nM |
| 10 V | 25 pmole/250 nM | 4 pmole/40 nM | 100 nM |

After conducting round 10V, binding of rounds 8V, 9V and 10V to immobilized fD was measured using flow cytometry as described above. All rounds exhibited binding to immobilized fD, with no apparent increase in the affinity of round 10V, as compared to round 9V, for fD. Subsequently, DNA pools from Fractions 1-4 of rounds 8V through 10V were analyzed by deep sequencing. Sequencing libraries for individual selection rounds were prepared using the archived primary PCR product as template and were amplified in a PCR reaction using forward and reverse library primers modified to include binding and barcoding sequences for multiplexed Illumina DNA sequencing. Sequencing reactions were run on an Illumina MiSeq sequencer using a 150 bp paired end read kit. Raw sequencing data consisted of paired-end sequence and read quality data in two FASTQ format files, one for each DNA strand.

Enrichment, defined as the frequency of a given sequence in a round divided by the total sequence count for the round was calculated for all sequences, and then the relative enrichment for each sequence present in round 10V fraction 1 (10V1) as compared to round 7 was calculated to identify those aptamers enriched in response to the selective pressure of elution using the clone of anti-fD Fab. For the top 1000 sequences, the relative enrichment of all sequenced rounds of selective pressure as well as round 8 of the primary selection was also calculated to compare the enrichment of these sequences across fraction 1 of selective pressure to their relative enrichment in the control fractions.

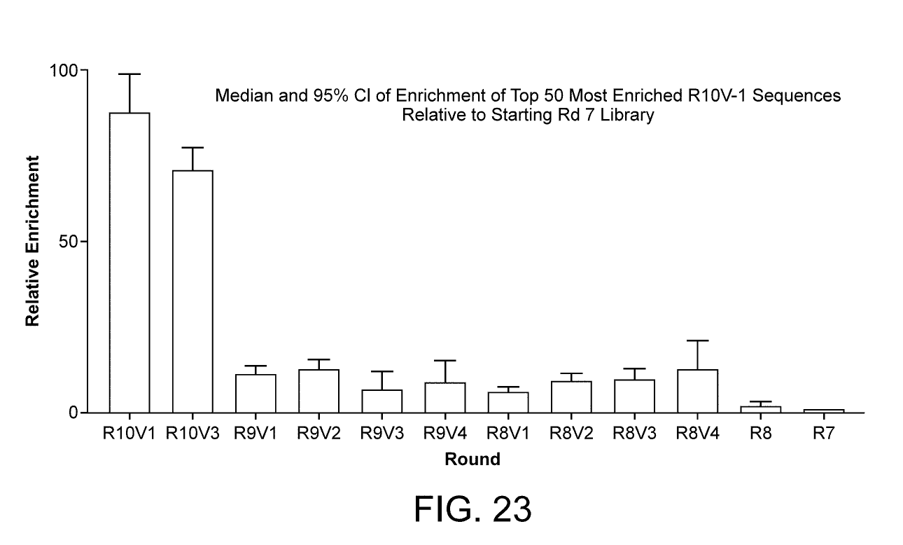

FIG. 23 shows a plot of the median relative enrichment with 95% confidence intervals of the top 50 most enriched aptamers in round 10V1 as compared to the other rounds of selective pressure as well as the last two rounds of the standard selection. Clearly, elution of aptamers with the clone of anti-fD Fab enriched for a population of aptamers not enriched in the primary selection. Importantly, this population of aptamers was enriched relative to the mock elution fraction 3 (FIG. 22), demonstrating that there is enrichment due to competition with the anti-fD Fab as opposed to simple equilibrium dissociation.

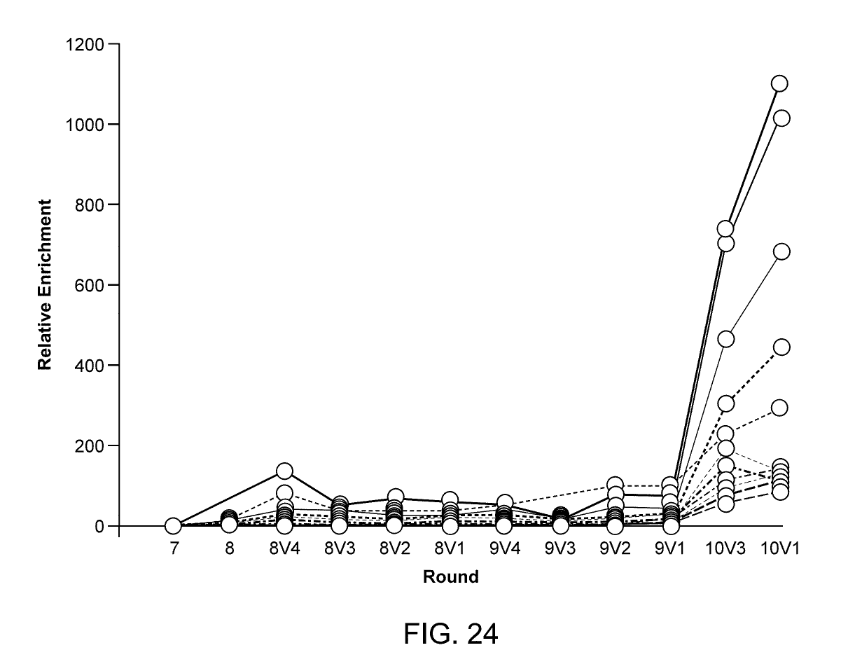

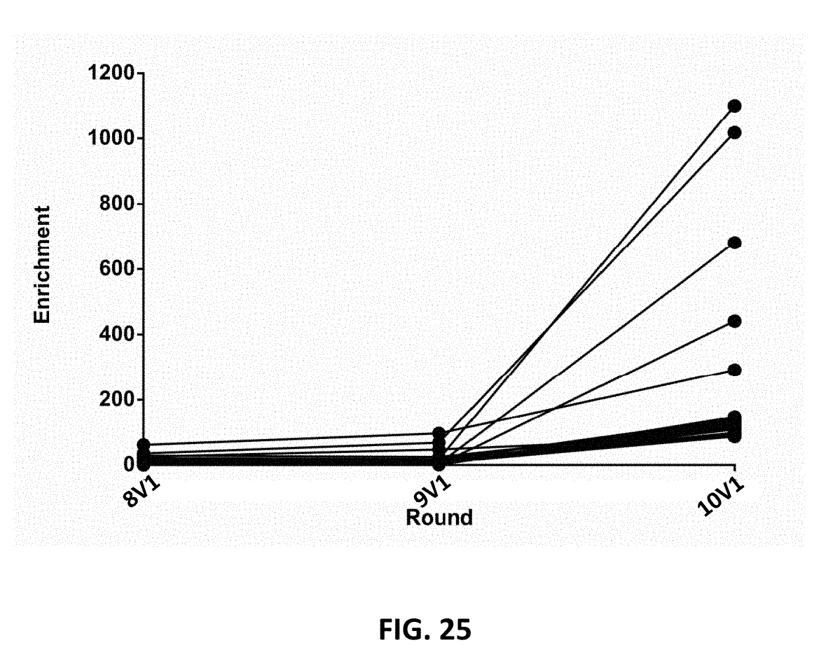

FIG. 24 shows individual enrichment plots of the top 25 most enriched aptamers in 10V1 (i.e., fraction competitively eluted). This analysis, in concordance with the population analysis presented in FIG. 23, shows a clear response in enrichment to the selective pressure presented by competitive elution with the clone of lamplizumab, with relative enrichments ranging from approximately 80-1100 fold over the 3 rounds of selective pressure. Further, as shown in the plot in FIG. 25, enrichment was modest from round 8V to 9V, and substantial in progressing from round 9V to 10V.

A critical feature of this improved selection method and informatics approach to analysis of the resultant aptamer library is that this population of aptamers would not have been identified by primary selection methods alone. The absolute frequency of the 50 most enriched aptamers present in 10V1 ranged from a high of 0.004 to <10⁻⁵ with a mean frequency of 0.0008 and median frequency of 7.2×10⁻⁵ for this population of aptamers, as depicted in Table 15.

TABLE 15
Sequence counts and frequency of 50 most enriched
aptamers in 10V1.

| | Sequence Counts | Frequency |
|---|---|---|
| Aptamer | R10V1 | R10V1 |
| 1 | 2117 | 0.000436 |
| 2 | 1957 | 0.000403 |
| 3 | 20957 | 0.004317 |
| 4 | 849 | 0.000175 |
| 5 | 561 | 0.000116 |
| 6 | 283 | 5.83E−05 |
| 7 | 542 | 0.000112 |
| 8 | 264 | 5.44E−05 |
| 9 | 515 | 0.000106 |
| 10 | 510 | 0.000105 |
| 11 | 254 | 5.23E−05 |
| 12 | 495 | 0.000102 |
| 13 | 233 | 4.80E−05 |
| 14 | 79775 | 0.016432 |
| 15 | 2642 | 0.000544 |
| 16 | 215 | 4.43E−05 |
| 17 | 209 | 4.31E−05 |
| 18 | 190 | 3.91E−05 |
| 19 | 557 | 0.000115 |
| 20 | 181 | 3.73E−05 |
| 21 | 3733 | 0.000769 |
| 22 | 355 | 7.31E−05 |
| 23 | 173 | 3.56E−05 |
| 24 | 169 | 3.48E−05 |
| 25 | 168 | 3.46E−05 |
| 26 | 167 | 3.44E−05 |
| 27 | 165 | 3.40E−05 |
| 28 | 329 | 6.78E−05 |
| 29 | 158 | 3.25E−05 |
| 30 | 308 | 6.34E−05 |
| 31 | 153 | 3.15E−05 |
| 32 | 458 | 9.43E−05 |
| 33 | 148 | 3.05E−05 |
| 34 | 292 | 6.01E−05 |
| 35 | 292 | 6.01E−05 |
| 36 | 15435 | 0.003179 |
| 37 | 6436 | 0.001326 |
| 38 | 142 | 2.92E−05 |
| 39 | 840 | 0.000173 |
| 40 | 15284 | 0.003148 |
| 41 | 1324 | 0.000273 |
| 42 | 1232 | 0.000254 |
| 43 | 349 | 7.19E−05 |
| 44 | 346 | 7.13E−05 |
| 45 | 1154 | 0.000238 |
| 46 | 311 | 6.41E−05 |
| 47 | 30500 | 0.006283 |
| 48 | 780 | 0.000161 |
| 49 | 556 | 0.000115 |
| 50 | 184 | 3.79E−05 |

In conclusion, this improved selection method and informatics approach has identified a population of aptamers competitively eluted by the anti-fD Fab, and which therefore are anticipated to contain individual aptamers that bind to the exosite of fD.

### Example 7. Modeling Positive Selection with Limited Target Concentration

As described herein, one method for increasing the stringency of a positive selection process and for preferentially enriching an aptamer pool for higher affinity binders, may involve limiting the amount of target molecule available for binding in a positive selection step. Under such conditions, without wishing to be bound by theory, aptamers with higher affinity may outcompete aptamers with lower affinity for the same epitope. This example demonstrates modeling of association curves of various compounds with different concentrations of their respective target molecules.

Briefly, binding constants provided in Table 16 were used with varying target concentrations to construct binding and kinetic association curves using one-site equilibrium binding models without ligand depletion and monophasic kinetic association curves (GraphPad Prism 7).

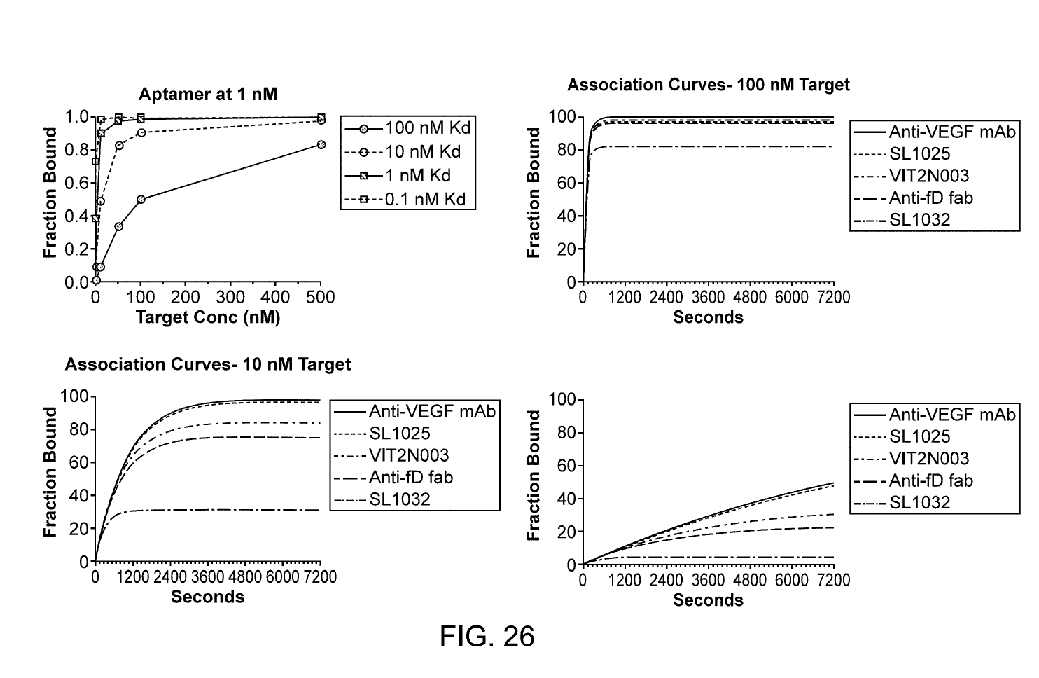

As depicted in FIG. 26, limiting target concentration readily eliminated low affinity binders. Amongst the higher affinity binders, kinetics and affinity impacted enrichment. The duration of the binding step in positive selection may be used to enrich for slower off-rate binders. Generally, the signal may need to be greater than background at low target concentration to ensure selection for target binders over non-specific binders. Additionally or alternatively, non-specific competitors as described herein may achieve enrichment similar to limiting target concentration.

### Example 8. Antibody Elution Modeling

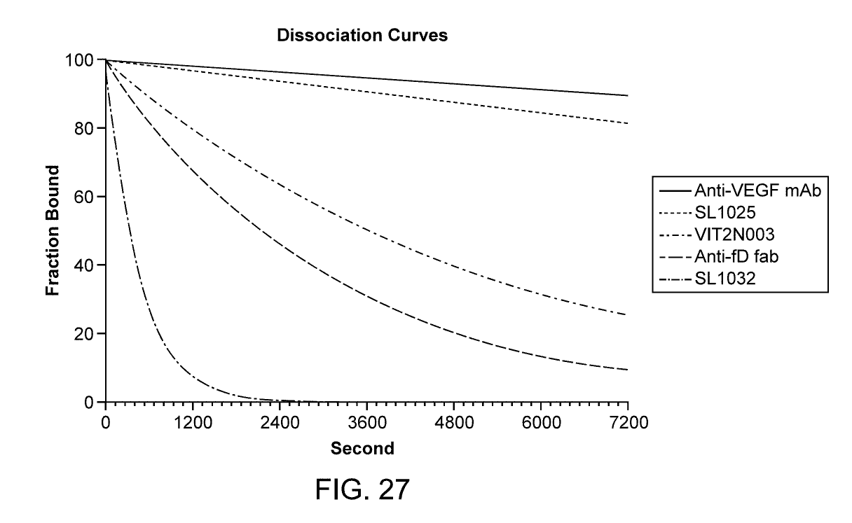

In some situations, it may be more challenging to recover those aptamers with highest affinity for the target molecule or those aptamers with slow off-rates. In such cases, the kinetics and affinity of the aptamers for the target epitope may need to be considered to ensure maximal recovery of desired aptamers. For example, in competitive elution methods, in order for an aptamer to be outcompeted by the competitor for the target molecule, the aptamer must initially dissociate from the target epitope. For slow off-rate aptamers, the incubation period of competitor with aptamer-target molecule complex may need to be longer to ensure the bound aptamer is able to dissociate. In such cases where the incubation period is not sufficient, slow off-rate aptamers may ultimately be lost. For example, Table 16 shows the t₁₁₂ of complexes formed between canonical aptamers and predicate mAbs and their respective targets and FIG. 27 depicts the respective dissociation curves. As shown, a therapeutically relevant mAb to the RBD of VEGF having an amino acid sequence of heavy chain variable region according to SEQ ID NO:3 and of light chain variable region according to SEQ ID NO:4 (anti-VEGF mAb) and SL1025 demonstrate very slow off-rates whereas SL1032 and VIT2N003 demonstrate very fast off-rates. In a competitive elution scheme as described herein, it may be necessary to increase the incubation time in order to allow enough time for dissociation of the desired aptamer. In the example depicted, a competitive elution time of 30 minutes may be sufficient time to allow dissociation of SL1032 from its target molecule, however, would be insufficient time to allow dissociation of anti-VEGF mAb.

TABLE 16
Kinetics of various antibodies and aptamers

| Compound | Kₒₙ (M⁻¹ s⁻¹) | K_{off} (s⁻¹) | K_{d} (nM) | T_{1/2} (min) |
|---|---|---|---|---|
| Anti-VEGF mAb | 3.0 × 10⁵ | 1.5 × 10⁻⁵ | 0.05 | 770 |
| Anti-fD Fab | 8.2 × 10⁷ | 3.3 × 10⁻⁴ | 0.004 | 35 |
| Anti-IL-6 aptamer | 1.2 × 10⁵ | 2.8 × 10⁻⁵ | 0.233 | 413 |
| #1 (SL1025) | | | | |
| Anti-IL-6 aptamer | 1.4 × 10⁶ | 2.2 × 10⁻³ | 1.6 | 5 |
| #2 (SL1032) | | | | |
| Anti-VEGF₁₂₁ | 6.39 × 10⁵ | 1.92 × 10⁻⁴ | 0.3 | 60 |
| aptamer #1 | | | | |
| (VIT2N003) | | | | |

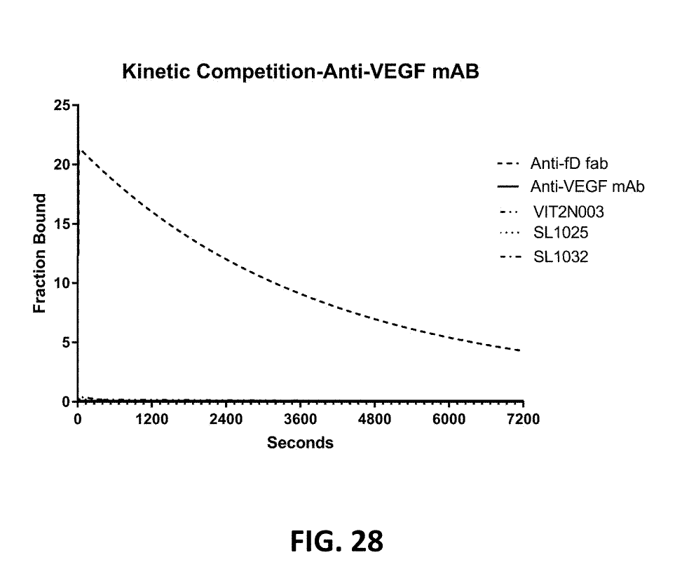

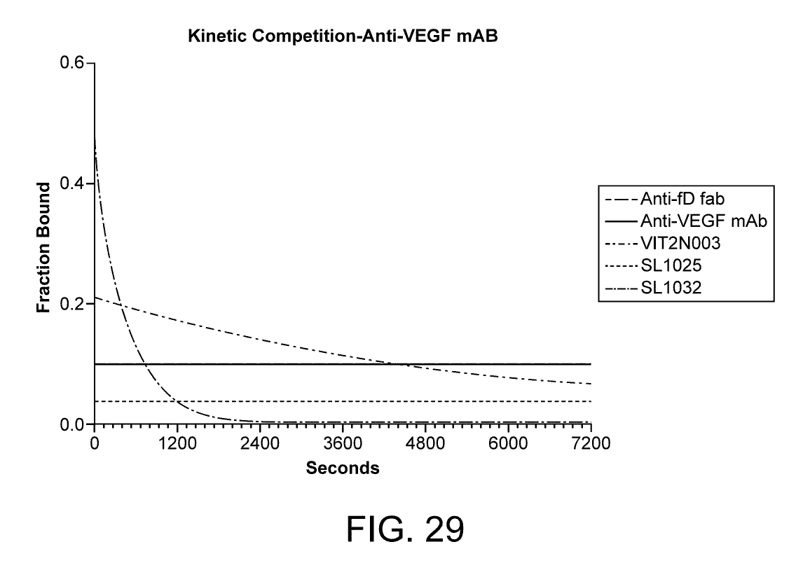

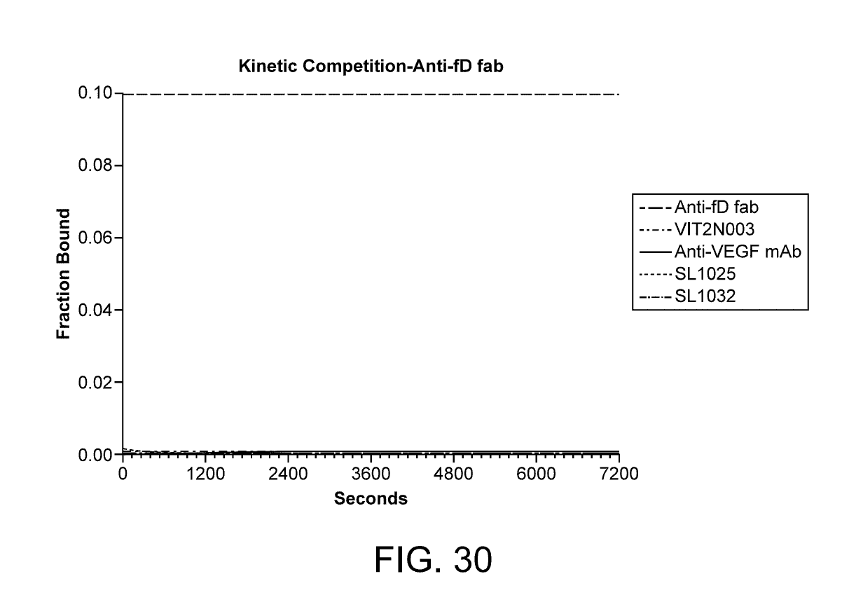

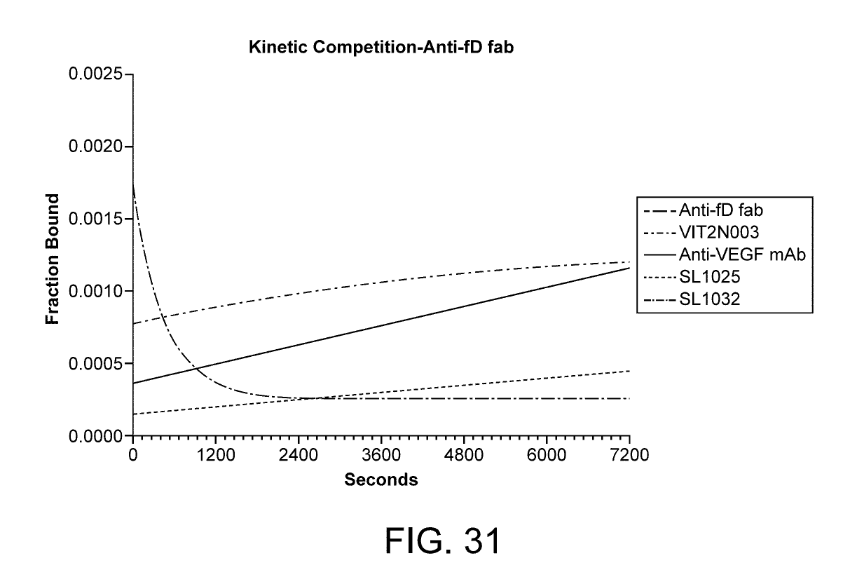

Briefly, Kinetics of Competitive Binding (Motulsky and Mahan Mol Pharmacology 25, p. 1-9 1984; pre-loaded in Prism 7.0) was used for simulations. The compound to be eluted was treated as the labeled ligand and the Y-axis depicts fraction bound of the compound. The predicate mAb was treated as the cold ligand. B_{Max} was set to 100 so Y-axis (Fraction Bound) was interpreted as % of compound bound at Time X; 100−Fraction Bound was the fraction eluted. Elution behavior of a given aptamer was interpreted as representative of a class of aptamers with similar binding properties. The predicate mAb was anti-VEGF mAb or anti-fD Fab at 1 μM. The concentration of desired aptamer was set at 1 nM. A lower concentration favored more effective elution. The mAb was assumed to be in molar excess (5-10 fold) of the target epitope. The model was viewed as a “jump ball” model which was initiated by dissociation of bound aptamers from desired epitope on target protein. As depicted in Tables 17 & 18, as well as FIGS. 28-31, all compounds were effectively eluted by anti-VEGF mAb and anti-fD Fab by 120 minutes.

TABLE 17
Fraction compound eluted by anti-VEGF mAb.

| | % Eluted | % Eluted | % Eluted |
|---|---|---|---|
| Compound | at 15 min | at 60 min | at 120 min |
| Anti-VEGF mAb | 99.90 | 99.90 | 99.90 |
| Anti-fD Fab | 82.80 | 90.90 | 95.72 |
| Anti-IL-6 aptamer #1 | 99.96 | 99.96 | 99.96 |
| (SL1025) | | | |
| Anti-IL-6 aptamer #2 | 99.93 | 99.99 | 99.99 |
| (SL1032) | | | |
| Anti-VEGF₁₂₁ | 99.82 | 99.89 | 99.93 |
| aptamer #1 | | | |
| (VIT2N003) | | | |

TABLE 18
Fraction compound eluted by anti-ID Fab

| | % Eluted | % Eluted | % Eluted |
|---|---|---|---|
| Compound | at 15 min | at 60 min | at 120 min |
| Anti-VEGF mAb | 99.99 | 99.99 | 99.99 |
| Anti-fD Fab | 99.90 | 99.90 | 99.90 |
| Anti-IL-6 aptamer #1 | 99.99 | 99.99 | 99.99 |
| (SL1025) | | | |
| Anti-IL-6 aptamer #2 | >>99.99 | >>99.99 | >>99.99 |
| (SL1032) | | | |
| Anti-VEGF₁₂₁ | 99.99 | 99.98 | 99.99 |
| aptamer #1 | | | |
| (VIT2N003) | | | |

Under these conditions even for very slow off-rate, very high affinity compounds like anti-VEGF mAb and SL1025 were effectively eluted upon dissociation from the target. Therefore, enrichment per round may largely be governed by dissociation of the desired aptamers from the epitope.

As demonstrated herein, the methods provided in this disclosure can effectively elute aptamers with wide range of affinity, regardless of underlying kinetics of aptamer binding. In some cases, 120-minute elution times using predicate mAb conditions described above may enable sufficient dissociation of very slow off-rate aptamers to enrich by iterating the selection process. The combination of counter-selection with target bound mAb followed by positive selection and competitive elution may drive selection for epitope-specific aptamers, and reduce the frequency of non-epitope binding aptamers in the pool. The kinetics of predicate mAb binding may need to be considered for each target. The most difficult class of compounds to elute may be compounds with a very high Kₒₙ coupled with very slow K_{off}. Decreasing the relative concentration of mAb:aptamer to 100:1 had a significant impact on elution vs. time, whereas increasing the relative concentration to 10,000:1 had no impact. Therefore, predicate mAb concentration should be ≥ or equal to the input aptamer library concentration (assuming desired aptamers are present at a frequency of ≤1/1000 within the library), and in some cases, 5-10× the input target protein concentration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for selecting a desired aptamer with high affinity for a target epitope of a target molecule, said method comprising:
a. obtaining an aptamer library comprising a plurality of aptamers;
b. contacting said aptamer library with a solid support such that non-specific aptamers bind to said solid support, thereby generating an aptamer library depleted of said non-specific aptamers;
c. incubating said aptamer library depleted of said non-specific aptamers with an isolated target molecule comprising said target epitope to form an aptamer-target molecule complex by binding of at least one aptamer of said aptamer library to said target epitope;
d. incubating said aptamer-target molecule complex with a competitor capable of specifically binding to said target epitope, thereby eluting said at least one aptamer from said target epitope;
e. recovering said at least one aptamer; and
f. iteratively repeating d)-e) one or more times, thereby selecting for a desired aptamer with high affinity for a target epitope of a target molecule,
wherein said at least one aptamer binds to said target epitope with a K_{d} of less than about 100 nM.

2. The method of claim 1, further comprising, prior to said incubating of c), immobilizing said target molecule to a solid support.

3.-9. (canceled)

10. A method for selecting for a desired aptamer that specifically binds to a target epitope with high affinity, said method comprising:
a. obtaining an aptamer library;
b. incubating said aptamer library with a target molecule comprising said target epitope to form an aptamer-target molecule complex by at least one aptamer binding to said target epitope;
c. incubating a competitor, capable of specifically binding to said target epitope, with said aptamer-target molecule complex at a ratio of at least 1000:1, thereby eluting said at least one aptamer from said target epitope; and
d. recovering said at least one aptamer, thereby selecting for a desired aptamer that specifically binds to a target epitope with high affinity.

11. The method of claim 10, further comprising, prior to b), immobilizing said target molecule to a solid support.

12. The method of claim 11, further comprising, prior to b),
contacting said aptamer library with a solid support in the absence of said target molecule to remove non-specific aptamers.

13. The method of claim 10, wherein c) further comprises providing said competitor at a high molar excess relative to said aptamer-target molecule complex.

14. The method of claim 13, further comprising performing two or more iterative rounds of c).

15. The method of claim 10, further comprising, repeating a)-d) one or more times, each time with a successively greater amount of competitor in c).

16. The method of claim 10, further comprising, prior to b), depleting non-target epitope binding aptamers from said aptamer library, comprising: i) incubating said target molecule with said competitor such that said competitor binds to said target epitope on said target molecule to generate a competitor-target molecule complex; ii) incubating said competitor-target molecule complex with said aptamer library such that non-target epitope binding aptamers bind to said competitor-target molecule complex and target epitope-binding aptamers do not bind to said competitor-target molecule complex; iii) collecting said target epitope-binding aptamers, thereby depleting non-target epitope binding aptamers from said aptamer library.

17. The method of claim 10, further comprising repeating c) one or more times in an iterative fashion.

18. The method of claim 17, wherein said repeating comprises incubating said aptamer-target molecule complex with successively greater amounts of competitor.

19. The method of claim 17, wherein said repeating comprises incubating said aptamer-target molecule complex with different competitors.

20.-23. (canceled)

24. A method of selecting for a desired aptamer that specifically binds to a target epitope on a target molecule with high affinity, the method comprising:
a. incubating target molecules comprising said target epitope with a non-competitive binder that blocks some, but not all, of said target epitopes on said target molecules;
b. obtaining an aptamer library comprising a number of aptamers capable of binding to said target epitope, wherein said number of aptamers capable of binding to said target epitope is greater than the number of unblocked epitopes and said number of aptamers have different binding affinities for said target epitope;
c. incubating said target molecules comprising blocked and unblocked target epitopes with said aptamer library such that said number of aptamers compete for binding to an unblocked target epitope, wherein an aptamer of said number of aptamers with higher affinity for said target epitope binds said target epitope and an aptamer of said number of aptamers with lower affinity for said target epitope does not bind said target epitope, thereby selecting for a desired aptamer that specifically binds to a target epitope with high affinity.

25. (canceled)

26. (canceled)

27. The method of claim 10, wherein said competitor is an antibody or antibody fragment thereof, a small molecule or a peptide.

28. The method of claim 10, wherein said competitor is a monoclonal antibody.

29.-32. (canceled)

33. The method of claim 10, wherein said desired aptamer binds to said target molecule with a K_{d} of about 100 nM or less.

34.-37. (canceled)

38. The method of claim 10, wherein said competitor is a therapeutic molecule used for the treatment of a target disease.

39. The method of claim 10, wherein said aptamer library comprises at least 10¹⁴ different aptamer sequences.

40. The method of claim 10, wherein said competitor is incubated with said aptamer-target molecule complex at a high molar excess.

41. (canceled)

42. The method of claim 10, wherein said target molecule is a recombinant protein or peptide.

43. The method of claim 10, wherein said aptamer library is a DNA aptamer library, a modified DNA aptamer library, an RNA aptamer library, or a modified RNA aptamer library.

44. (canceled)

45. (canceled)
